(19) United States
(12) Patent Application Publication
(10) Pub. No.: US 2024/0091382 A1
(43) Pub. Date: Mar. 21, 2024

(54) MINIMAL BILE ACID INDUCIBLE PROMOTERS FOR GENE THERAPY

(71) Applicant: VIVET THERAPEUTICS, Paris (FR)

(72) Inventors: Gloria GONZALEZ ASEGUINOLAZA, Barañain (ES); Cristian SMERDOU, Pamplona (ES)

(21) Appl. No.: 18/268,680

(22) PCT Filed: Dec. 22, 2021
(86) PCT No.: PCT/EP2021/087437

§ 371 Date: Jun. 21, 2023

(30) Foreign Application Priority Data
Dec. 23, 2020 (EP) 20306684.0

(51) Int. Cl. A61K 48/00; A61P 1/16; C07K 14/47; C12N 15/86

(52) U.S. Cl. CPC A61K 48/0058; A61P 1/16; C07K 14/47; C12N 15/86; C12N 2750/14143; C12N 2800/22; C12N 2830/002; C12N 2830/50

(57) ABSTRACT

The present disclosure is in the field of gene therapy, in particular for the treatment of cholestatic disease. More specifically, the present invention relates to a minimal bile acid inducible promoter and its use for gene therapy in cholestatic disease.

### FIELD OF THE INVENTION

The present disclosure is in the field of gene therapy, in particular for the treatment of cholestatic diseases. More specifically, the present invention relates to a minimal bile acid inducible promoter and its use for gene therapy in cholestatic diseases.

### BACKGROUND ART

Intrahepatic cholestasis results from impairment of bile secretion and accumulation of bile salts in the organism. Hereditary monogenic cholestatic diseases are characterized by impaired bile secretion and accumulation of bile salts in the organism. Hereditary cholestasis is a heterogeneous group of rare autosomal recessive liver disorders, which are characterized by intrahepatic cholestasis, pruritus, and jaundice and caused by defects in genes related to the secretion and transport of bile salts and lipids. Phenotypic manifestation is highly variable, ranging from progressive familial intrahepatic cholestasis (PFIC)—with onset in early infancy and progression to end-stage liver disease—to a milder intermittent mostly nonprogressive form known as benign recurrent intrahepatic cholestasis (BRIC). Cases have been reported of initially benign episodic cholestasis that subsequently transitions to a persistent progressive form of the disease. Therefore, BRIC and PFIC seem to represent two extremes of a continuous spectrum of phenotypes that comprise one disease. Thus far, five representatives of PFIC (named PFIC1-5) caused by pathogenic mutations present in both alleles of ATP8B1, ABCB11, ABCB4, TJP2, and NR1H4 have been described (Sticova et al., Canadian Journal of Gastroenterology and Hepatology (2018)).

These diseases include, among others, progressive familial intrahepatic cholestasis type 2 (PFIC2) caused by mutations in the ABCB11 gene coding for bile salt export pump (BSEP) and progressive familial intrahepatic cholestasis type 3 (PFIC3), associated with mutations in the ABCB4 gene coding for multidrug resistance protein 3 (MDR3) (Davit-Spraul, A et al. Orphanet J Rare Dis 4 (2009)).

In the case of PFIC2, BSEP deficiency causes bile acids to accumulate in the liver, leading to hepatocyte death, bile release to the blood, severe pruritus, liver cirrhosis and failure, and ultimately death in untreated patients within the first few years of life. Additionally, affected individuals are at increased risk of developing hepatocellular carcinoma at an early age. In the case of PFIC3, mutations in MDR3 decrease the transport of phosphatidylcholine to the bile, which prevents proper micelle formation, resulting in bile canaliculi and biliary epithelium injury, leading to cholestasis.

Current therapeutic options for PFIC2 and PFIC3 patients include administration of ursodeoxycholic acid (UDCA), a non-toxic hydrophilic bile acid that can partially replace toxic hydrophobic bile salts (Jacquemin, E., et al. Hepatology 25, 519-523 (1997)). Although 50% of PFIC3 patients respond well to this therapy, most PFIC2 patients do not respond, requiring other therapeutic options. These include surgical methods based on biliary diversion, which decrease the enterohepatic circulation of bile salts leading to the reduction of toxic bile salt accumulation. Although these methods can ameliorate the life of PFIC2 and PFIC3 patients, liver transplant is in fact the only available curative therapy for PFIC2 patients and for approximately half of PFIC3 patients. However, liver transplants carry with them the risks involved with such a complicated procedure, as well as a chance of re-emergence of the condition (van der Woerd W L et al. World J Gastroenterol. 23 (5):763-775 (2017)).

Currently, gene therapy vectors based on adeno associated virus (AAV) have been shown to be highly efficacious for the treatment of genetic liver diseases (Baruteau, J. et al. J Inherit Metab Dis 40, 497-517 (2017)). In the case of PFIC3, AAV vectors expressing MDR3 downstream of a constitutive liver-specific promoter (alpha 1 antitrypsin promoter; A1T) have shown to be able to revert disease symptoms in a clinically relevant mouse model of this disease (Weber N. D. et al. Nature Communications. 13; 10 (1):5694 (2019)). However, using gene therapy, MDR3 was expressed at constant levels, therefore not reproducing endogenous regulation, since MDR3 expression is naturally regulated by bile salts concentration (Gupta S, et al. Hepatology. 32:341-347 (2000)). A physiologically regulated expression of the defective gene underlying the disease (e.g. MDR3 for PFIC3, BSEP for PFIC2) is highly desirable to allow to strictly reproduce a close-to endogenous level of expression based on physiological need. Such temporal expression could be achieved by expressing these transgenes using their endogenous promoters, leading to high or appropriate expression levels only when needed.

However, both BSEP and MDR3 promoters are very large (1-5-2 kb) (Ananthanarayanan M et al. J Biol Chem. 3; 276 (31):28857-65 (2001); Huang L et al. Biol Chem.; 278 (51):51085-90 (2003)), which, together with the large size of their respective transgenes (approximately 4 kb) results in promoter and transgene cassettes that are too large to be packaged into AAV vectors, which have a maximum packaging capacity of approximately 4.7 kb (Dong J Y, Fan P D, Frizzell R A. Hum Gene Ther.; 7 (17):2101-12 (1996)).

Thus, there remains a need to develop small promoters regulated by bile salts concentration while allowing an efficient expression of the transgene for gene therapy of cholestatic disorders.

### SUMMARY OF THE INVENTION

The promoters of human and mouse BSEP genes were cloned and different regulatory elements that mediate bile acid-induced gene expression changes were identified (Ananthanarayanan M et al. J Biol Chem.; 276 (31):28857-65 (2001); Song X. et al. J Lipid Res.; 54 (11):3030-44 (2013); Song X et al. J Lipid Res.; 49 (5):973-84 (2008); Gerloff et al. Eur. J. Biochem.; 269:3495-3503 (2002)). By using a minimal version of the endogenous BSEP promoter from mouse origin comprising only the last 145 nucleotides of the promoter followed by the 5′ untranslated region of BSEP mRNA, the inventors unexpectedly showed better and inducible gene expression using a reporter gene than with the corresponding human version, in human hepatic cells. Similar results were obtained in mouse hepatic cells as well as in the liver of mice. Interestingly, AAV vectors having genes coding for BSEP or MDR3 downstream of the minimal mouse BSEP promoter were able to control or revert disease symptoms in mouse models of PFIC2 and PFIC3, respectively.

The present invention relates to a nucleic acid construct comprising a minimal bile acid inducible promoter having a length of less than 500 bp and comprising or consisting of nucleic acid sequence SEQ ID NO: 1 or a functional variant thereof having at least 95% identity to SEQ ID NO: 1 operably linked to a therapeutic transgene. In a particular embodiment, the nucleic acid construct can further comprise 5′ and 3′ ITR sequences of an adeno-associated virus, preferably said nucleic acid construct comprises a 5′ITR and a 3′ITR sequences from the AAV2 serotype, more preferably of SEQ ID NO: 11 and 12, respectively. In another particular embodiment said nucleic acid construct comprises a poly(A) sequence, preferably a poly(A) signal sequence of SEQ ID NO: 10. In a particular embodiment, said promoter further comprises at least one murine IR-1 element, preferably of SEQ ID NO: 3, preferably said promoter comprises or consists of nucleotide sequence SEQ ID NO: 6 or 7, more preferably SEQ ID NO: 7. In a particular embodiment, said promoter has a length of less than 450 bp, preferably 400 bp, 350 bp, 300 bp, more preferably 250 bp. In a preferred embodiment, said promoter is operably linked to a therapeutic transgene encoding for human BSEP, preferably said transgene is a codon optimized sequence encoding for BSEP, more preferably said transgene comprises or consists of SEQ ID NO: 8 or a variant having at least 80% identity to SEQ ID NO: 8. In another preferred embodiment, said promoter is operably linked to a therapeutic transgene encoding for human MDR3 protein, preferably said transgene is a codon optimized sequence encoding for MDR3 protein, more preferably said transgene comprises or consists of SEQ ID NO: 9 or a variant having at least 80% identity to SEQ ID NO: 9.

In another aspect, the present invention relates to an expression vector comprising a nucleic acid construct as described above and a viral particle comprising said nucleic acid construct or expression vector. In a particular embodiment, the present invention relates to an AAV particle comprising a nucleic acid construct or expression vector as described above and preferably comprising capsid proteins of adeno-associated virus such as capsid proteins selected from the group consisting of: AAV3 type 3A, AAV3 type 3B, NP40, NP59, NP84, LK03, AAV3-ST, Anc80 and AAV8 serotype, preferably AAV8.

In another aspect, the present invention relates to a host cell comprising a nucleic acid construct or an expression vector as described above or transduced with a viral particle as described above.

The present invention also relates to a pharmaceutical composition comprising a nucleic acid construct, an expression vector, a viral particle or a host cell as described above and a pharmaceutically acceptable excipient. The present invention relates to a nucleic acid construct, an expression vector, a viral particle, a host cell or a pharmaceutical composition as described above for its use as a medicament in a subject in need thereof, preferably for the prevention and/or treatment of cholestatic diseases, preferably Progressive Familial Intrahepatic Cholestasis such as Progressive Familial Intrahepatic Cholestasis Type 2 (PFIC2) or Progressive Familial Intrahepatic Cholestasis Type 3 (PFIC3).

Finally, the present invention relates to a method of producing viral particles as described above, comprising the steps of: a) culturing a host cell comprising a nucleic acid construct or an expression vector as described above in a culture medium, and b) harvesting the viral particles from the cell culture supernatant and/or inside the cells.

### BRIEF DESCRIPTION OF THE DRAWINGS

FIG. 1. Diagram of vectors expressing luciferase downstream of BSEP derived promoters. ITR, inverted terminal repeats; pA, synthetic polyadenylation signal. imPr and ihPr, minimal mouse and human BSEP promoters, respectively; mBSEPpr and hBSEPpr, full-length mouse and human BSEP promoters, respectively.

FIG. 2. Bile acid induction of luciferase expression in human hepatic cell lines. Huh7 (A-C) and HepG2 cells (D) were transfected with vectors expressing luciferase downstream of the promoters indicated with color code in A-C, and 24h later incubated for 30 h with 30 μM CDCA (p+CDCA 30 μM) or with 0.3% DMSO (Luc plasmid). In addition, some cells were also co-transfected with plasmids expressing the indicated human FXR isoforms (FXRα2 in A, B and D and FXRα1 in C) and incubated with 30 μM CDCA (p+FXR+CDCA 30 μM) or with 0.3% DMSO (p+FXR). In all cases, cells were also transfected with a Renilla expressing plasmid (pRL-CMV). Luciferase and Renilla expression were measured with a luminometer. All samples were tested in triplicates and data are presented as mean±SEM of relative (rel.) units (Luciferase units sec⁻¹/Renilla units sec⁻¹). The fold induction of each condition relative to cells transfected only with the luciferase plasmid (Luc plasmid) is indicated on top of each column. The statistical comparison between these two conditions was performed by using an unpaired 30 T test. *, p<0.05; **, p<0.01; ***, p<0.001. imPr and ihPr, pAAV-imPr-LucPEST and pAAV-ihPr-LucPEST, respectively; full length mPr, pAAV-mBSEPpr-LucPEST; full length hPr, p-2563/+4-Luc; A1AT, pAAV-A1AT-LucPEST.

FIG. 3. Bile acid induction of luciferase expression in a murine hepatic cell line. Hepa 1-6 cells were transfected as indicated with vectors expressing luciferase downstream of the promoters indicated with color code in A, and 24 h later incubated for 30 h with 100 μM CDCA (p+CDCA 100 μM) or with 1% DMSO (Luc plasmid). In addition, some cells were also cotransfected with plasmids expressing the indicated human FXR isoforms (FXRα2 in A and FXRα1 in B) and incubated with 100 μM CDCA (p+FXR+CDCA 100 μM) or with 0.1% DMSO (p+FXR). In all cases, cells were also transfected with a Renilla expressing plasmid (pRL-CMV). Luciferase and Renilla expression were measured with a luminometer. All samples were tested in triplicates and data are presented as mean±SEM of relative (rel.) units (Luciferase units sec⁻¹/Renilla units sec⁻¹). The fold induction of each condition relative to cells transfected only with the luciferase plasmid (Luc plasmid) is indicated on top of each column. The statistical comparison between these two conditions was performed by using an unpaired T test. *, p<0.05; **, p<0.01; ***, p<0.001. imPr and ihPr, pAAV-imPr-LucPEST and pAAV-ihPr-LucPEST, respectively; full length mPr, pAAV-mBSEPpr-LucPEST; full length hPr, p-2563/+4-Luc; A1AT, pAAV-A1AT-LucPEST.

FIG. 4. UDCA and OCA induction of luciferase expression in human hepatic cells. Huh7 cells were transfected with pAAV-imPr-LucPEST (imPr) and 24 h later incubated for 30 h with OCA or UDCA at the indicated concentrations or with DMSO (Mock). In addition, some cells were also co-transfected with plasmids expressing human FXRα2 and incubated with CDCA, OCA, or UDCA (pLuc+FXRα2). All samples were tested in triplicates and data are presented as mean+SEM of relative (rel.) units (Luciferase units sec⁻¹/Renilla units sec⁻¹). The fold induction of each condition relative to cells transfected only with the luciferase plasmid (pLuc) is indicated on top of each column, as well as the statistical analysis comparing the two conditions by using a Student T test. **, p<0.01; *** p<0.001.

FIG. 5. Bile salt induction of minimal BSEP promoters in C57BL/6 mouse model. C57BL/6 female and male mice were inoculated with 3×10¹² VG (viral genomes)/kg of AAV-imPr-LucPEST (A), AAV-A1AT-LucPEST (B) and AAV-ihPr-LucPEST (C) and received normal diet or a diet supplemented with 0.2% cholic acid (CA) during the three-week intervals indicated with gray-shaded rectangles. Luciferase was measured in live mice at the indicated times (n=3 for each gender except in mice receiving AAV-ihPr-LucPEST where n=2, only tested in females). Data are shown as mean±SEM. imPr, AAV-imPr-LucPEST; ihPr, AAV-ihPr-LucPEST; A1AT, AAV-AA1T-LucPEST. Ph/s, photons/second.

FIG. 6. Luciferase expression in Abcb4 KO and WT mice. FVB Abcb4 KO and WT female and male mice were intravenously injected with 3×10¹² VG/kg of AAV-imPr-LucPEST (A), AAV8-A1AT-LucPEST (B) and AAV-ihPr-LucPEST (C). Luciferase was measured in live mice at the indicated times (n=3 for each gender and vector, except in KO males inoculated with AAV-imPr-LucPEST where n=4). Data are shown as mean±SEM. imPr, AAV-imPr-LucPEST; ihPr, AAV-ihPr-LucPEST; A1AT, AAV-A1AT-LucPEST.

FIG. 7: AAV transduction and transgene expression for Abcb4 KO and WT mice treated with AAV8-LucPEST. The levels of LucPEST mRNA transcripts (a) and AAV genomes (b) were quantified from liver tissue harvested from Abcb4 KO and WT mice treated with AAV8-imPr-LucPEST, AAV8-ihPr-LucPEST, and AAV8-A1AT-LucPEST at 3×10¹² VG/kg. AAV genomes and LucPEST transcripts were quantified via qPCR and RT-qPCR, respectively. Animals were sacrificed at 22 weeks after treatment. The graphs show the mean+SEM values of all mice, including males and females (since no significant differences were observed between genders). *, p<0.05; **, p<0.01. imPr, AAV8-imPr-LucPEST; ihPr, AAV8-ihPr-LucPEST; A1AT, AAV8-A1AT-LucPEST.

FIG. 8: Bile salt levels in Abcb4 KO and WT mice. Mice were bled at the indicated times post-injection and the content of total bile salts (BS) in serum was measured. The graphs show the mean+SEM values of all mice (including males and females since no significant differences were observed between genders), treated with 3×10¹² VG/kg of AAV8-imPr-LucPEST (a), AAV8-A1AT-LucPEST (b), or AAV8-ihPr-LucPEST (c). *, p<0.05; **, p<0.01; ***, p<0.001. imPr, AAV8-imPr-LucPEST; ihPr, AA8V-ihPr-LucPEST; A1AT, AAV8-A1AT-LucPEST.

FIG. 9. Diagram of vectors expressing BSEP and MDR3 downstream of imPr promoter. ITR, inverted terminal repeats; pA, synthetic polyadenylation signal. imPr, minimal mouse B SEP promoter.

FIG. 10. Analysis of AAV-imPr-hBSEPco expression in Abcb11 KO mice. 4-week-old Abcb11 KO mice were intravenously injected with a dose of 6×10¹³ VG/kg of AAV8-imPr-hBSEPco (two males and three females), or saline, and sacrificed one week later. Liver extracts were analyzed to determine AAV genomes (A) and BSEP mRNA (B) by qPCR and RT-qPCR, respectively ΔCt corresponds to Ct for house-keeping gene GAPDH—Ct for human ABCB11 codon optimized cDNA. Data are shown as mean+SEM. (C) Immunohistochemistry of liver sections stained with an anti-BSEP-specific antibody (Santa Cruz). Representative pictures from one mouse in each group are shown. M, male, F, female.

FIG. 11. Analysis of serum biomarkers, hepatomegaly and fibrosis in Abcb11 KO mice treated with AAV-imPr-hBSEPco. Four-week old Abcb11 KO female mice were treated with 6×10¹³ VG/kg of AAV-imPr-hBSEPco (imPr) or AAV-A1AT-co-hBSEP (A1AT) or left untreated and the levels of ALT and AST (A), and bilirubin (B) were determined in serum at the indicated mice ages (n≥5 in all groups for each age). Data show mean+SEM. Empty bars correspond to basal levels in treated mice. (C) Liver weight was determined at the time of sacrifice when mice were six months old. (D) Analysis of fibrosis by picrosirius red staining of liver sections. In the left panel, images of two representative mice untreated and treated with AAV-imPr-hBSEPco are shown. The degree of fibrosis was quantified using a FIJI V1.46b plugins (ImageJ) for image analysis as the percentage of stained areas compared to WT mice (right graph). The statistical analysis was performed using a Mann-Whitney test. *, p<0.05; **, p 21 0.01; ns, non-significant.

FIG. 12. AAV liver transduction in Abcb11 KO mice. AAV viral DNA (A) and hBSEP mRNA (B) levels were quantified in liver extracts from the same mice described in FIG. 11 by qPCR or RT-qPCR, respectively, after sacrifice. ΔCt corresponds to Ct for house-keeping gene GAPDH—Ct for human ABCB11 codon optimized cDNA. (C) BSEP expression was analyzed by immunohistochemistry (IHC) of liver sections using a BSEP specific antibody (LS-Bio). One representative mouse from each treatment group is shown. NT, non-treated, A1AT, AAV-A1AT-hBSEPco; imPr, AAV-imPr-hBSEPco. Data show mean±SEM. The statistical analysis was performed using a Mann-Whitney test. ns, non-significant.

FIG. 13. Analysis of AAV-imPr-MDR3co expression in Abcb4 KO mice. Two-week-old Abcb4 KO male mice were intravenously injected with AAV-imPr-MDR3co at different doses (10¹⁴ or 1.5×10¹⁴ VG/kg) (n=3 for each dose) or with AAV-A1AT-MDR3(A)co at a dose of 10¹⁴ VG/kg (n=2) or with saline (n=2). (A) AAV genomes and MDR3 expression in liver extracts were analyzed at one (one mouse per group and dose) and two weeks (n=2 for AAV8-imPr-MDR3(A)co and n=1 for control groups) post inoculation (pi) by qPCR and RT-qPCR, respectively, using in both cases oligonucleotides specific for hMDR3co. (B) Liver sections were stained with a MDR3-specific antibody (LS-Bio) and the percentage of MDR3 expression was determined as the percentage of positive tissue area with respect to the signal observed in WT mice using FIJI V1.46b plugins (ImageJ) program. Data are shown as mean+SEM. (C) Representative images of MDR3-stained liver sections of one mouse from each group.

FIG. 14. Analysis of serum biomarkers in Abcb4 KO mice treated with AAV-imPr-MDR3co. Five-week-old Abcb4 KO male mice were treated with 10¹⁴ VG/kg of AAV8-imPr-MDR3co (n=3) or AAV8-A1AT-MDR3(A)co (n=4) or with saline (n=4). (A) The levels of ALT (alanine aminotransferase), AST (aspartate aminotransferase), bile salts, and bilirubin were determined in serum at the indicated times after treatment. (B) Liver weight was determined at the time of sacrifice when mice were three months old. Data show mean±SEM.

FIG. 15. Analysis of fibrosis in Abcb4 KO mice treated with AAV8-imPr-MDR3co. The same mice presented in FIG. 14 were sacrificed at three months of age. Liver was extracted and fibrosis was analyzed by picrosirius red staining in liver sections or each mouse. (A) In the left panel, representative images of non-responders from one mouse in each AAV-treated group or saline-treated Abcb4 KO mice are shown. In the right panel, representative images of responder Abcb4 KO mice. Scale bar=300 μm. (B) The degree of fibrosis was quantified using a FIJI V1.46b plugins (ImageJ) for image analysis. Data are presented as mean±SEM.

FIG. 16. MDR3 expression quantification in Abcb4 KO mice treated with AAV8-imPr-MDR3co. Liver sections from mice treated as indicated in FIG. 14 were stained with an MDR3-specific antibody and expression was analyzed by IHC. (A) In the left panel, representative images of non-responders from one mouse in each AAV-treated group or saline-treated Abcb4 KO mice are shown. In the right panel, representative pictures of responders Abcb4 KO mice and the quantification of the amount of MDR3 staining are shown. Scale bar=100 μm (in each picture the area indicated by the smaller square is magnified in the larger square). (B) The percentage of MDR3 expression was determined as the percentage of positive tissue area with respect to the signal observed in WT mice using a FIJI V1.46b plugin (ImageJ) for image analysis. Data are presented as mean±SEM.

FIG. 17. Biliary PC in Abcb4 KO mice treated with AAV8-imPr-MDR3co. PC concentration in bile was measured at three months of age for Abcb4 KO mice treated as indicated in FIG. 14. Data are presented as mean±SEM. Statistical analysis was performed by using an unpaired Student T test. *, p<0.05.

FIG. 18. Bile acid induction of luciferase expression of new imPr versions. Huh7 cells were transfected with pAAV plasmids expressing LucPEST downstream of the promoters indicated with color code and 24 h later incubated for 30 h with 30 μM CDCA (p+CDCA 30 μM) or with 0.3% DMSO (Luc plasmid). In addition, some cells were also co-transfected with plasmids expressing FXRα2 and incubated with 30 μM CDCA (p+FXR+CDCA 30 μM) or with 0.3% DMSO (p+FXR). In all cases, cells were also transfected with the same amount of Renilla expressing plasmid (pRL-CMV) to determine the transfection efficiency. Luciferase and Renilla expression were measured with a luminometer. All samples were tested in triplicates and data are presented as mean±SEM of relative (rel.) units (Luciferase units sec⁻¹/Renilla units sec⁻¹). The fold induction of each condition relative to cells transfected only with the luciferase plasmid (Luc plasmid) is indicated on top of each column. The statistical comparison between these two conditions was performed by using an unpaired T test. *, p<0.05; **, p<0.01; ***, p<0.001; ****, p<0.0001.

FIG. 19. Bile acid induction of an optimized minimal BSEP promoter in wild type mice. C57BL/6 male and female mice were administered 3×10¹² VG/kg of AAV8-imPr-3xIR-LucPEST or AAV8-imPr-LucPEST and received either a diet supplemented with 0.2% CA (closed symbols) alternating with normal diet (open symbols) (a) or a continuous normal diet (b). Luciferase was measured in live mice at the indicated times (n=4). Data are shown as mean+SEM of photon (Ph) units per second normalized to background signal. Statistical comparisons were calculated comparing the average luciferase expression during each CA cycle and its precedent normal diet cycle using a paired T test. *, p<0.05; **, p<0.01. Fold induction in each CA cycle was calculated by dividing the maximum expression under CA induction by basal expression prior to CA administration. (indicated in the upper part of the graph for imPr-3xIR and in the lower part for imPr). imPr, AAV8-imPr-LucPEST; imPr-3xIR, AAV8-imPr-3xIR-LucPEST.

### DETAILED DESCRIPTION

### Minimal Bile Acid Inducible Promoter

In the present disclosure, by using a minimal version of the endogenous murine BSEP promoter (Genebank accession number: AF190697.1 submitted on Jun. 3, 2001) comprising only the last 145 nucleotides of the promoter followed by the 5′ untranslated region of murine BSEP mRNA, the inventors unexpectedly showed better expression and inducibility of a reporter gene than the corresponding human version in human hepatic cells. Similar results were obtained in mouse hepatic cells as well as in the liver of mice. They showed that AAV vectors having genes coding for BSEP or MDR3 downstream of the minimal mouse BSEP promoter were able to control or revert disease symptoms in mouse models of PFIC2 and PFIC3.

The present disclosure relates to a minimal bile acid inducible promoter having a length of less than 500 bp promoter and comprising or consisting of the SEQ ID NO: 1 or functional variants thereof.

The minimal bile acid inducible promoter of SEQ ID NO: 1 was derived from the regulatory region of the murine ATP-binding cassette, subfamily B (MDR/TAP), member 11 gene, also known as Bsep gene (Symbol: Abcb11 Gene ID: 27413). This gene encodes a member of the MDR/TAP subfamily which is liver resident transporter protein which plays an essential role in the enterohepatic circulation of the bile salts. Mutations in this gene cause a form of progressive familial intrahepatic cholestasis which are a group of inherited disorders with severe cholestatic liver disease from early infancy. The gene is located on chromosome 2 (from position 69,068,626 to position 69,172,960, GRCm39 (genome reference consortium (June 2020), Ref Seq GCF_000001635.27).

The promoter of SEQ ID NO: 1 comprises the last 145 nucleotides of the endogenous BSEP promoter followed by the 5′ untranslated region of BSEP mRNA. In another embodiment, the promoter of the invention comprises or consists of the nucleotide sequence from position 69,172,868 to position 69,173,089 of chromosome 2, GRCm39, Ref Seq CGF 000001635.27.

As used herein, the term “promoter” refers to a regulatory element that directs the transcription of a nucleic acid to which it is operably linked. A promoter can regulate both rate and efficiency of transcription of an operably linked nucleic acid. A promoter may also be operably linked to other regulatory elements which enhance (“enhancers”) or repress (“repressors”) promoter-dependent transcription of a nucleic acid. These regulatory elements include, without limitation, transcription factor binding sites, repressor and activator protein binding sites, and any other sequences of nucleotides known to one of skill in the art to act directly or indirectly to regulate the amount of transcription from the promoter, including e.g. attenuators, enhancers, and silencers. The promoter is located near the transcription start site of the gene or coding sequence to which it is operably linked, on the same strand and upstream of the DNA sequence (towards the 5′ region of the sense strand). A promoter can be about 100-1000 base pairs long. Positions in a promoter are designated relative to the transcriptional start site for a particular gene (i.e., positions upstream are negative numbers counting back from −1, for example −100 is a position 100 base pairs upstream).

The term “minimal promoter” refers to the minimal portion of a promoter sequence required to properly initiate transcription. The minimal promoter refers to a minimal sequence that contains all the transcriptional start sites and transcription factor-binding sites allowing the expression of the operably linked transgene. In particular, it includes the transcription start site (TSS) and elements directly upstream, a binding site for RNA polymerase (RNA polymerase II); and general transcription factors binding sites. In particular, minimal bile acid inducible promoter comprises an IR-1 element and ER2 motif binding sites. Commonly a promoter also comprises a proximal promoter sequence (upstream of the core promoter), that contains other primary regulatory elements (such as enhancers, silencers, boundary elements/insulators); and a distal promoter sequence (downstream of core promoter), that may contain additional regulatory elements, normally with a weaker influence on the level of transcription of the gene. In particular, the minimal promoter according to the invention is devoid of distal promoter sequence elements.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a promoter or transcription regulatory sequence is operably linked to a coding sequence if it affects the transcription of the coding sequence. Operably linked means that the DNA sequences being linked are typically contiguous; where it is necessary to join two protein encoding regions, they are contiguous and in reading frame.

In the context of the invention, the minimal bile acid inducible promoter according to the disclosure exhibits a promoter activity in liver cells and exhibits a higher promoter activity than human minimal bile acid promoter of SEQ ID NO: 2 in liver cells. Typically, the activity of the minimal bile acid inducible promoter according to the disclosure will be at least 1,5, at least 2, at least 3, at least 4, at least 5 or at least 10 times more active than the activity of the human minimal bile acid promoter of SEQ ID NO: 2 in the liver cells.

As used herein, the term “promoter activity” refers to the ability of a promoter to initiate transcription of a nucleic acid to which it is operably linked. Promoter activity can be measured using procedures known in the art or as described in the Examples. For example, promoter activity can be measured as an amount of mRNA transcribed by using, for example, Northern blotting or polymerase chain reaction (PCR). Alternatively, promoter activity can be measured as an amount of translated protein product, for example, by Western blotting, ELISA, colorimetric assays and various activity assays, including reporter gene assays and other procedures known in the art or as described in the examples. To test promoter activity, the promoter may be operably linked to a screenable marker and introduced into a host cell. The expression level of the screenable marker may be assessed and the promoter activity may be determined based on the level of expression of the screenable marker. The biological activity of the promoter may be determined either visually or quantitatively based on levels of screenable marker expression in host cells.

In a particular embodiment, the present disclosure relates to a promoter comprising or consisting of a functional variant of SEQ ID NO: 1.

As used herein, the term “variant” refers to a nucleotide sequence differing from the original sequence but retaining essential properties thereof. Generally, variants are overall closely similar, and, in many regions, identical to the original polynucleotide. The sequence of the variant may differ by nucleotide substitutions, deletions or insertions of one or more nucleotides in the sequence, which do not impair the promoter activity. The variant may have the same length of the original sequence or may be shorter or longer.

The term “functional variant” refers to a variant of SEQ ID NO: 1 that exhibits a promoter activity of SEQ ID NO: 1, i.e. that exhibits a higher promoter activity in liver cells than human minimal bile acid promoter of SEQ ID NO: 2.

In a particular embodiment, the promoter of the invention comprises or consists of a functional variant having at least 90, 91, 92, 93, 94, 95, 96, 97, 98, 99% identity to SEQ ID NO: 1, preferably over the entire sequence of SEQ ID NO: 1.

As used herein, the term “sequence identity” or “identity” refers to the number of matches (identical nucleic acid residues) in positions from an alignment of two polynucleotide or polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithms (e.g. Needleman and Wunsch algorithm; Needleman and Wunsch, J Mol Biol.; 48 (3):443-53 (1970)) which aligns the sequences optimally over the entire length, while sequences of substantially different lengths are preferably aligned using a local alignment algorithm (e.g. Smith and Waterman algorithm (Smith and Waterman, J Theor Biol; 91 (2):379-80 (1981)) or Altschul algorithm (Altschul S F et al., Nucleic Acids Res; 25 (17):3389-402. (1997); Altschul S F et al., Bioinformatics; 21 (8):1451-6 (2005)). Alignment for purposes of determining percent nucleic acid or polypeptide sequence identity can be achieved in various ways that are within the skill in the art, for instance, using publicly available computer software available on internet web sites such as http://blast.ncbi.nlm.nih.gov/ or http://www.ebi.ac.uk/Tools/emboss/). Those skilled in the art can determine appropriate parameters for measuring alignment, including any algorithms needed to achieve maximal alignment over the full length of the sequences being compared. For purposes herein, % nucleic acid or polypeptide sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

The promoter of the invention may differ from the nucleic acid sequence of SEQ ID NO: 1 by 1, 2, 3, 4, 5, 6, 7, 8, 9, 10, 12, 13, 14, 15 substitutions, deletions and/or insertions.

The promoter of the invention may further comprise regulatory elements. These regulatory elements include, without limitation, transcription factor binding sites, repressor and activator protein binding sites, and any other sequences of nucleotides known to one of skill in the art to act directly or indirectly to regulate the amount of transcription from the promoter, including e.g. attenuators, enhancers, and silencers.

In a particular embodiment, said promoter as described above may further comprise at least one inverted repeats (IR) element, preferably of SEQ ID NO: 3.

The inverted repeats (IR) element acts as a binding element for the transcription factor FXR (Ananthanarayanan M et al. J Biol Chem. 276 (31):28857-65 (2001); Song X. et al. J Lipid Res. 54 (11):3030-44 (2013)). According to the invention said IR element is an IR-1 element which is a sequence composed of two inverted repeats separated by one nucleotide, preferably a murine IR-1 element of SEQ ID NO: 3 (TTAGGCCATTGACCTA).

In a particular embodiment, the nucleotide sequence comprising the IR-1 element can further comprise an everted repeat separated by two nucleotides (ER2) motif, preferably of sequence TGGACT which is necessary to achieve maximum FXR transactivation (Song X. et al. J Lipid Res. 2013 November; 54 (11):3030-44). Thus, in a particular embodiment, said promoter further comprises at least one nucleotide sequence comprising IR element and an ER2 motif, preferably of SEQ ID NO: 4 (TGGACTTTAGGCCATTGACCTA).

In another particular embodiment, the promoter according to the invention further comprise Liver receptor homolog 1 responsive element (LRHRE), preferably of SEQ ID NO: 5 (TTTCTAAAGCT). Liver receptor homolog 1 (LRH-1) transcriptionally regulates the expression of BSEP promoter through a functional LRHRE in the BSEP promoter and functions as a modulator in bile acid/FXR-mediated BSEP regulation (Song X et al. J Lipid Res.; 49 (5):973-84 (2008)). According to the disclosure, said LRHRE is a murine LRHRE, preferably of SEQ ID NO: 5.

In a preferred embodiment, the promoter according to the invention is a minimal bile acid inducible promoter comprising a nucleic acid sequence of SEQ ID NO: 1 and at least one nucleic acid sequence of SEQ ID NO: 3 or functional variants thereof.

In a preferred embodiment, said promoter comprises or consists of a nucleic acid sequence of SEQ ID NO: 1 and one nucleic acid sequence of SEQ ID NO: 3, preferably said promoter comprises or consists of SEQ ID NO: 6.

In a more preferred embodiment, said promoter comprises or consists of a nucleic acid sequence of SEQ ID NO: 1 and at least two, preferably three nucleic acid sequences of SEQ ID NO: 3, preferably said promoter comprises or consists of SEQ ID NO: 7.

In another particular embodiment, the promoter of the invention comprises, or consists of, a functional variant having a sequence comprising at least 50, 60, 70, 80, 90, 100, 110, 120, 130, 140, 150, 160, 170, 180, 190, 200 consecutive nucleotides of SEQ ID NO: 1.

The promoter of the invention is small and has a length of less than 500 bases, preferably of less than 450, 400, 450, 300 bases, more preferably 250 bases and is particularly suitable for use in AAV vectors wherein the DNA payload is limited.

### Nucleic Acid Construct

In another aspect, the present invention relates to a nucleic acid construct comprising a promoter as described above operably linked to a transgene.

The terms “nucleic acid sequence” and “nucleotide sequence” may be used interchangeably to refer to any molecule composed of or comprising monomeric nucleotides. A nucleic acid may be an oligonucleotide or a polynucleotide. A nucleotide sequence may be a DNA or RNA. A nucleotide sequence may be chemically modified or artificial. Nucleotide sequences include peptide nucleic acids (PNA), morpholinos and locked nucleic acids (LNA), as well as glycol nucleic acids (GNA) and threose nucleic acid (TNA). Each of these sequences is distinguished from naturally-occurring DNA or

RNA by changes to the backbone of the molecule. Also, phosphorothioate nucleotides may be used. Other deoxynucleotide analogs include methylphosphonates, phosphoramidates, phosphorodithioates, N3′P5′-phosphoramidates and oligoribonucleotide phosphorothioates and their 2′-0-allyl analogs and 2′-0-methylribonucleotide methylphosphonates which may be used in a nucleotide of the invention.

The term “nucleic acid construct” as used herein refers to a man-made nucleic acid molecule resulting from the use of recombinant DNA technology. A nucleic acid construct is a nucleic acid molecule, either single- or double-stranded, which has been modified to contain segments of nucleic acids sequences, which are combined and juxtaposed in a manner, which would not otherwise exist in nature. A nucleic acid construct usually is a “vector”, i.e. a nucleic acid molecule which is used to deliver exogenously created DNA into a host cell.

As used herein, the term “transgene” refers to exogenous DNA or cDNA encoding a gene product. The gene product may be an RNA, peptide or protein. In addition to the coding region for the gene product, the transgene may include or be associated with one or more elements to facilitate or enhance expression, such as a promoter, enhancer(s), response element(s), reporter element(s), insulator element(s), polyadenylation signal(s) and/or other functional elements.

As used herein, said nucleic acid construct comprises a promoter as described above operably linked to a transgene. Generally, the nucleic acid construct comprises a coding sequence and regulatory sequences preceding (5′ non-coding sequences) and following (3′ non-coding sequences) the coding sequence that are required for expression of the selected transgene product. Thus, a nucleic acid construct typically comprises a promoter sequence, a coding sequence and a 3′ untranslated region that usually contains a polyadenylation site and/or transcription terminator. The nucleic acid construct may also comprise additional regulatory elements such as, for example, enhancer sequences, a polylinker sequence facilitating the insertion of a DNA fragment within a vector and/or splicing signal sequences. In certain embodiments, the nucleic acid construct according to the invention only comprises as 5′ eukaryotic regulatory element operably linked to a transgene, the minimal bile acid promoter as described in the previous sequence, typically consisting of SEQ ID NO: 1, 6 or 7 or functional variants thereof. As used herein, eukaryotic regulatory element is a segment of a nucleic acid molecule which is capable of increasing or decreasing the expression of genes within an eukaryotic organism. In particular, in such embodiment, the nucleic acid construct is devoid of distal elements of the promoter, such as enhancer sequences.

More particularly, the present invention relates to a nucleic acid construct comprising a transgene encoding a polypeptide of interest, in particular any polypeptide of which bile salt inducible expression in cells is desired. In a more particular embodiment, said transgene is a therapeutic transgene. In particular, said transgene encodes a protein involved in the bile acid metabolism pathway.

As used herein, the term “therapeutic transgene” refers to a gene encoding a therapeutic RNA, peptide or protein which is useful in the treatment of a pathological condition. The therapeutic transgene when expressed, confers a beneficial effect on the cell or tissue in which it is present, or on a patient in which the gene is expressed. Examples of beneficial effects include amelioration of a sign or symptom of a condition or disease, prevention or inhibition of a condition or disease, or conferral of a desired characteristic. Therapeutic genes include genes that partially or wholly correct a genetic deficiency in the patient. In particular, the therapeutic gene may be, without limitation, a nucleic acid sequence encoding a protein useful in gene therapy to relieve deficiencies caused by missing, defective or sub-optimal levels of said protein in a cell or tissue of a subject. The therapeutic polypeptide may, e.g., supply a polypeptide and/or enzymatic activity that is absent, defective or present at a suboptimal level in cells, supply a polypeptide and/or enzymatic activity that indirectly counteracts an imbalance in cells. The therapeutic polypeptide may also be used to reduce the activity of a polypeptide by acting, e.g., as a dominant-negative polypeptide.

Preferably, the therapeutic polypeptide supplies a polypeptide and/or enzymatic activity that is absent, defective or present at a sub-optimal level in liver cells, more preferably a polypeptide and/or enzymatic activity that is absent or defective in liver cells.

In another embodiment, the transgene may also encode a nucleic acid, for example, an siRNA, an shRNA an RNAi, an miRNA, an antisense RNA, a ribozyme or a DNAzyme. In a particular embodiment, the nucleic acid encodes an RNA that when transcribed from the nucleic acid operably linked to the promoter of the invention can treat or prevent a disease, preferably a cholestasis disease by interfering with translation or transcription of an abnormal or excess protein associated with said disorder. For example, the nucleic acid of interest may encode for an RNA, which treats the disease by highly specific elimination or reduction of mRNA encoding the abnormal and/or excess proteins.

Examples of therapeutic transgenes include, but are not limited to, nucleic acids for replacement of a missing or mutated gene known to cause cholestatic disease such as MDR3, BSEP, FIC1, or ABCG5/G8, preferably native mammalian MDR3 or BSEP, more preferably human MDR3 (NCBI reference sequence NP_000434.1), human BSEP (NCBI reference sequence: NP_003733.2) or variants thereof.

The membrane-associated protein MDR3 encoded by ABCB4 gene, also named MDR3 gene is a member of the superfamily of ATP-binding cassette (ABC) transporters. This gene encodes a full transporter and member of the p-glycoprotein family of membrane proteins with phosphatidylcholine as its substrate which may be involved in transport of phospholipids from liver hepatocytes into bile. Alternative splicing of this gene results in three potential human isoforms, designated A (NCBI reference sequence NP_000434.1), B (NCBI reference sequence NP_061337.1) and C (NCBI reference sequence NP_061338.1). In a preferred embodiment, said transgene encodes MDR3 isoform A (NCBI reference sequence NP_000434.1).

The membrane-associated protein BSEP encoded by ABCB11 gene is a member of the superfamily of ATP-binding cassette (ABC) transporters. This gene encodes a liver resident transporter protein which plays an essential role in the enterohepatic circulation of the bile salts (NCBI reference sequence: NP_003733.2).

Preferably, as used herein, the term “variant” refers to a polypeptide having an amino acid sequence having at least 70, 75, 80, 85, 90, 95 or 99% sequence identity to the native sequence.

More preferably, the term “variant” refers to a polypeptide having an amino acid sequence that differs from a native sequence by less than 30, 25, 20, 15, 10 or 5 substitutions, insertions and/or deletions. In a preferred embodiment, the variant differs from the native sequence by one or more conservative substitutions, preferably by less than 15, 10 or 5 conservative substitutions. Examples of conservative substitutions are within the groups of basic amino acids (arginine, lysine and histidine), acidic amino acids (glutamic acid and aspartic acid), polar amino acids (glutamine and asparagine), hydrophobic amino acids (methionine, leucine, isoleucine and valine), aromatic amino acids (phenylalanine, tryptophan and tyrosine), and small amino acids (glycine, alanine, serine and threonine). MDR3 or BSEP activity of a variant may be assessed by any method known by the skilled person as described above.

The coding sequences of a number of different mammalian MDR3 or BSEP proteins are known including, but being not limited to, human, pig, chimpanzee, dog, cow, mouse, rabbit or rat, and can be easily found in sequence databases. Alternatively, the coding sequence may be easily determined by the skilled person based on the polypeptide sequence.

In a particular embodiment said transgene may be an optimized sequence encoding MDR3 or BSEP protein or variants thereof.

The term “codon optimized” means that a codon that expresses a bias for human (i.e. is common in human genes but uncommon in other mammalian genes or non-mammalian genes) is changed to a synonymous codon (a codon that codes for the same amino acid) that does not express a bias for human. Thus, the change in codon does not result in any amino acid change in the encoded protein. In a particular embodiment, said transgene is an optimized sequence encoding B SEP, preferably of SEQ ID NO: 8. In another particular embodiment, said transgene is an optimized sequence encoding MDR3, preferably of SEQ ID NO: 9.

In some embodiments, the promoter according to the disclosure is not operably linked to murine or human ABCB11 gene. In some other embodiments, the promoter of the invention is not operably linked to a transgene encoding a reporter protein.

Each of these nucleic acid construct embodiments may also include a polyadenylation signal sequence; together or not with other optional nucleotide elements. As used herein, the term “polyadenylation signal” or “poly(A) signal” refers to a specific recognition sequence within 3′ untranslated region (3′ UTR) of the gene, which is transcribed into precursor mRNA molecule and guides the termination of the gene transcription. Poly(A) signal acts as a signal for the endonucleolytic cleavage of the newly formed precursor mRNA at its 3′-end, and for the addition to this 3′-end of a RNA stretch consisting only of adenine bases (polyadenylation process; poly(A) tail). Poly(A) tail is important for the nuclear export, translation, and stability of mRNA. In the context of the invention, the polyadenylation signal is a recognition sequence that can direct polyadenylation of mammalian genes and/or viral genes, in mammalian cells.

Poly(A) signals typically consist of a) a consensus sequence AAUAAA, which has been shown to be required for both 3′-end cleavage and polyadenylation of premessenger RNA (pre-mRNA) as well as to promote downstream transcriptional termination, and b) additional elements upstream and downstream of AAUAAA that control the efficiency of utilization of AAUAAA as a poly(A) signal. There is considerable variability in these motifs in mammalian genes.

In one embodiment, the polyadenylation signal sequence of the nucleic acid construct of the invention is a polyadenylation signal sequence of a mammalian gene or a viral gene. Suitable polyadenylation signals include, among others, a SV40 early polyadenylation signal, a SV40 late polyadenylation signal, a HSV thymidine kinase polyadenylation signal, a protamine gene polyadenylation signal, an adenovirus 5 EIb polyadenylation signal, a growth hormone polyadenylation signal, a PBGD polyadenylation signal, in silico designed polyadenylation signal (synthetic) and the like. In a particular embodiment, the polyadenylation signal sequence of the nucleic acid construct is a synthetic poly(A) signal sequence based on the rabbit beta-globin gene, more particularly a synthetic poly(A) having sequence SEQ ID NO: 10.

In a particular embodiment, the nucleic acid construct according to the present invention comprises the minimal bile acid inducible promoter comprising or consisting of SEQ ID NO: 1, a therapeutic transgene encoding BSEP, preferably consisting of SEQ ID NO: 8, and poly(A) sequence of SEQ ID NO: 10.

In another particular embodiment, the nucleic acid construct according to the present invention comprises the minimal bile acid inducible promoter comprising or consisting of SEQ ID NO: 6 or 7, a therapeutic transgene encoding BSEP, preferably consisting of SEQ ID NO: 8, and poly(A) sequence of SEQ ID NO: 10.

In another particular embodiment, the nucleic acid construct according to the present invention comprises the minimal bile acid inducible promoter comprising or consisting of SEQ ID NO: 1, a therapeutic transgene encoding MDR3, preferably of SEQ ID NO: 9, and poly(A) sequence of SEQ ID NO: 10.

In another particular embodiment, the nucleic acid construct according to the present invention comprises the minimal bile acid inducible promoter comprising or consisting of SEQ ID NO: 6 or 7, a therapeutic transgene encoding MDR3, preferably of SEQ ID NO: 9, and poly(A) sequence of SEQ ID NO: 10.

### Expression Vector

The nucleic acid construct of the invention may be comprised in an expression vector. As used herein, the term “expression vector” refers to a nucleic acid molecule used as a vehicle to transfer genetic material, and in particular to deliver a nucleic acid into a host cell, either in vitro or in vivo. Expression vector also refers to a nucleic acid molecule capable of effecting expression of a gene (transgene) in host cells or host organisms compatible with such sequences. Expression vectors typically include at least suitable transcription regulatory sequences and optionally 3′-transcription termination signals. Additional factors necessary or helpful in effecting expression may also be present, such as expression enhancer elements able to respond to a precise inductive signal (endogenous or chimeric transcription factors) or specific for certain cells, organs or tissues. Vectors include, but are not limited to, plasmids, phasmids, cosmids, transposable elements, viruses, and artificial chromosomes (e.g., YACs). Preferably, the vector of the invention is a vector suitable for use in gene or cell therapy, and in particular is suitable to target liver cells.

In some embodiments, the expression vector is a viral vector, such as retroviral vectors derived from Moloney murine leukemia virus vectors (MoMLV), MSCV, SFFV, MPSV, SNV, Harvey murine sarcoma virus vectors, murine mammary tumor virus vectors or Rous sarcoma virus vector; lentiviral vectors (e.g. derived from human immunodeficiency virus (HIV), simian immunodeficiency virus (SIV), feline immunodeficiency virus (FIV), bovine immunodeficiency virus (BIV) or equine infectious anemia virus (EIAV)), adenoviral (Ad) vectors, adeno-associated viral (AAV) vectors, simian virus 40 (SV-40) vectors, bovine papilloma virus vectors, Epstein-Barr virus, herpes virus vectors or vaccinia virus vectors.

As is known in the art, depending on the specific viral vector considered for use, suitable sequences should be introduced into the vector of the invention for obtaining a functional viral vector, such as AAV ITRs for an AAV vector, or LTRs for lentiviral vectors. In a particular embodiment, said vector is an AAV vector.

AAV has arisen considerable interest as a potential vector for human gene therapy. Among the favourable properties of the virus are its lack of association with any human disease, its ability to infect both dividing and non-dividing cells, and the wide range of cell lines derived from different tissues that can be infected. The AAV genome is composed of a linear, single-stranded DNA molecule which contains 4681 bases (Berns and Bohenzky, 1987, Advances in Virus Research (Academic Press, Inc.) 32:243-307). The genome includes inverted terminal repeats (ITRs) at each end, which function in cis as origins of DNA replication and as packaging signals for the virus. The ITRs are approximately 145 bp in length. The internal non-repeated portion of the genome includes two large open reading frames, known as the AAV rep and cap genes, respectively. These genes code for the viral proteins involved in replication and packaging of the virion. In particular, at least four viral proteins are synthesized from the AAV rep gene, Rep 78, Rep 68, Rep 52 and Rep 40, named according to their apparent molecular weight. The AAV cap gene encodes at least three proteins, VP1, VP2 and VP3. For a detailed description of the AAV genome, see, e.g., Muzyczka, N. Current Topics in Microbiol. and Immunol. 158:97-129 (1992).

Thus, in one embodiment, the nucleic acid construct or expression vector comprising transgene of the invention further comprises a 5′ITR and a 3′ITR sequences, preferably a 5′ITR and a 3′ ITR sequences of an adeno-associated virus.

As used herein the term “inverted terminal repeat (ITR)” refers to a nucleotide sequence located at the 5′-end (5′ITR) and a nucleotide sequence located at the 3′-end (3′ITR) of a virus, that contain palindromic sequences and that can fold over to form T-shaped hairpin structures that function as primers during initiation of DNA replication. They are also needed for viral genome integration into the host genome; for the rescue from the host genome; and for the encapsidation of viral nucleic acid into mature virions. The ITRs are required in cis for the vector genome replication and its packaging into the viral particles.

AAV ITRs for use in the viral vector of the invention may have a wild-type nucleotide sequence or may be altered by the insertion, deletion or substitution. The serotype of the inverted terminal repeats (ITRs) of the AAV may be selected from any known human or nonhuman AAV serotype. In specific embodiments, the nucleic acid construct or viral expression vector may be carried out by using ITRs of any AAV serotype, including AAV1, AAV2, AAV3 (including types 3A and 3B), AAV4, AAV5, AAV6, AAV7, AAV8, AAV9, AAV10, AAV11, AAV12, avian AAV, bovine AAV, canine AAV, equine AAV, ovine AAV, and any other AAV serotype now known or later discovered.

In one embodiment, the nucleic acid construct can be designed to be self-complementary AAV (scAAV). “Self-complementary AAV” refers to AAV vector designed to form an intra-molecular double-stranded DNA template which does not require DNA synthesis (D M McCarty et al. 2001. Gene Therapy, 8 (16):1248-1254). Upon infection, rather than waiting for cell mediated synthesis of the second strand, the two complementary halves of scAAV will associate to form one double stranded DNA (dsDNA) unit that is ready for immediate replication and transcription. For example, the AAV may be engineered to have a genome comprising two connected single-stranded DNAs that encode, respectively, a transgene unit and its complement, which can snap together following delivery into a target cell, yielding a double-stranded DNA encoding the transgene unit of interest. Self-complementary AAVs are described in for instance U.S. Pat. Nos. 6,596,535; 7,125,717 and 7,456,683.

In one embodiment, the nucleic acid construct further comprises a 5′ITR and a 3′ITR of an AAV of a serotype AAV2, preferably a 5′ITR and 3′ITR of SEQ ID NO: 11 and 12, respectively.

In one embodiment, the nucleic acid construct or AAV vector genome according to the invention is comprised in a recombinant baculovirus genome. As used herein, the term “recombinant baculovirus genome” refers to a nucleic acid that comprises baculoviral genetic elements for autonomous replication of a recombinant baculovirus genome in a host cell permissive for baculovirus infection and replication, typically insect cells. The term “recombinant baculovirus genome” expressly includes genomes comprising nucleic acids that are heterologous to the baculovirus. Likewise, the term “recombinant baculovirus genome” does not necessarily refer to a complete baculovirus genome as the genome may lack viral sequences that are not necessary for completion of an infection cycle. In particular, the recombinant baculovirus genomes may include the heterologous AAV genes useful for rAAV production and/or the transgene to be encapsidated in the rAAV for use in gene therapy. The baculoviral genetic elements for use in the present disclosure are preferably obtained from AcMNPV baculovirus (Autographa californica multinucleocapsid nucleopolyhedrovirus).

In a particular embodiment, the genes encoding baculovirus cathepsin and chitinase in said first and second baculoviral genomes are disrupted or deleted. In particular, the genes v-cath (Ac127) and chiA (Ac126) of the AcMNPV baculovirus may be disrupted or deleted so that the corresponding cathepsin or chitinase are either not expressed or expressed as inactive forms (i.e. have no enzymatic cathepsin or chitinase activity). In a particular embodiment, said recombinant baculovirus genomes are further disrupted or deleted for at least p24 gene (Ac129), preferably for the three baculoviral genes p10 (Ac137), p24 and p26 (Ac136). In a particular embodiment, said recombinant baculovirus genomes include functional p74 baculoviral gene (Ac138) (i.e. said gene has not been deleted or disrupted).

On the other hand, the nucleic acid construct or expression vector of the invention can be carried out by using synthetic 5′ITR and/or 3′ITR; and also by using a 5′ITR and a 3′ITR which come from viruses of different serotypes. All other viral genes required for viral vector replication can be provided in trans within the virus-producing cells (packaging cells) as described below. Therefore, their inclusion in the viral vector is optional.

In one embodiment, the nucleic acid construct or viral vector of the invention comprises a 5′ITR, a ψ packaging signal, and a 3′ITR of a virus. “ψ packaging signal” is a cis-acting nucleotide sequence of the virus genome, which in some viruses (e.g. adenoviruses, lentiviruses . . . ) is essential for the process of packaging the virus genome into the viral capsid during replication.

The construction of recombinant AAV viral particles is generally known in the art and has been described for instance in U.S. Pat. Nos. 5,173,414 and 5,139,941; WO 92/01070, WO 93/03769, Lebkowski et al. Molec. Cell. Biol. 8:3988-3996 (1988); Vincent et al. Vaccines 90, Cold Spring Harbor Laboratory Press, (1990); Carter, B. J. Current Opinion in Biotechnology 3:533-539 (1992); Muzyczka, N. Current Topics in Microbiol. and Immunol. 158:97-129 (1992); and Kotin, R. M. Human Gene Therapy 5:793-801 (1994).

In a particular embodiment, the expression vector according to the present invention comprises the minimal bile acid inducible promoter comprising or consisting of SEQ ID NO: 1, a therapeutic transgene encoding BSEP, preferably of SEQ ID NO: 8, poly(A) sequence of SEQ ID NO: 10 and 5′ITR and 3′ITR of SEQ ID NO: 11 and 12, respectively.

In another particular embodiment, the expression vector according to the present invention comprises the minimal bile acid inducible promoter comprising or consisting of SEQ ID NO: 6 or 7, a therapeutic transgene encoding BSEP, preferably of SEQ ID NO: 8, poly(A) sequence of SEQ ID NO: 10 and 5′ITR and 3′ITR of SEQ ID NO: 11 and 12, respectively.

In a particular embodiment, the expression vector according to the present invention comprises the minimal bile acid inducible promoter comprising or consisting of SEQ ID NO: 1, a therapeutic transgene encoding MDR3, preferably of SEQ ID NO: 9, poly(A) sequence of SEQ ID NO: 10 and 5′ITR and 3′ITR of SEQ ID NO: 11 and 12, respectively.

In another particular embodiment, the expression vector according to the present invention comprises the minimal bile acid inducible promoter comprising or consisting of SEQ ID NO: 6 or 7; a therapeutic transgene encoding MDR3, preferably of SEQ ID NO: 9, poly(A) sequence of SEQ ID NO: 10 and 5′ITR and 3′ITR of SEQ ID NO: 11 and 12, respectively.

In a particular embodiment, the expression vector of the invention comprises or consists of SEQ ID NO: 13 or 14 or a sequence having at least 70, 75, 80, 85, 90, 95, 96, 97, 98, 99% of identity with SEQ ID NO: 13 or 14.

### Viral Particle

The nucleic acid construct or the expression vector of the invention may be packaged into a virus capsid to generate a “viral particle”, also named “viral vector particle”. In a particular embodiment, the nucleic acid construct or the expression vector of the invention is packaged into an AAV-derived capsid to generate an “adeno-associated viral particle” or “AAV particle”. The present invention relates to a viral particle comprising a nucleic acid construct or an expression vector of the invention and preferably comprising capsid proteins of adeno-associated virus.

The term AAV vector particle encompasses any recombinant AAV vector particle or mutant AAV vector particle, genetically engineered. A recombinant AAV particle may be prepared by encapsidating the nucleic acid construct or viral expression vector including ITR(s) derived from a particular AAV serotype on a viral particle formed by natural or mutant Cap proteins corresponding to an AAV of the same or different serotype.

Proteins of the viral capsid of an adeno-associated virus include the capsid proteins VP1, VP2, and VP3. Differences among the capsid protein sequences of the various AAV serotypes result in the use of different cell surface receptors for cell entry. In combination with alternative intracellular processing pathways, this gives rise to distinct tissue tropisms for each AAV serotype.

Several techniques have been developed to modify and improve the structural and functional properties of naturally occurring AAV viral particles (Bunning H et al. J Gene Med. 10: 717-733 (2008); Paulk et al. Mol Ther. 26 (1):289-303 (2018); Wang L et al. Mol Ther. 23 (12):1877-87 (2015); Vercauteren et al. Mol Ther. 24 (6):1042-1049 (2016); Zinn E et al., Cell Rep. 12 (6):1056-68 (2015)).

Thus, in AAV viral particle according to the present disclosure, the nucleic acid construct or viral expression vector including ITR(s) of a given AAV serotype can be packaged, for example, into: a) a viral particle constituted of capsid proteins derived from the same or different AAV serotype [e.g. AAV2 ITRs and AAV5 capsid proteins; AAV2 ITRs and AAV8 capsid proteins; AAV2 ITRs and Anc80 capsid proteins; AAV2 ITRs and AAV9 capsid proteins]; b) a mosaic viral particle constituted of a mixture of capsid proteins from different AAV serotypes or mutants [e.g. AAV2 ITRs with AAV1 and AAV5 capsid proteins]; c) a chimeric viral particle constituted of capsid proteins that have been truncated by domain swapping between different AAV serotypes or variants [e.g. AAV2 ITRs with AAV5 capsid proteins with AAV3 domains].

The skilled person will appreciate that the AAV viral particle for use according to the present disclosure may comprise capsid proteins from any AAV serotype including AAV1, AAV2, AAV3 (including types 3A and 3B), AAV4, AAV5, AAV6, AAV7, AAV8, AAV9, AAV10, AAV11, AAV12, avian AAV, bovine AAV, canine AAV, equine AAV, ovine AAV, synthetic AAV variants such as NP40, NP59, NP84 (Paulk et al. Mol Ther. 26 (1):289-303 (2018)), LK03 (Wang L et al. Mol Ther. 23 (12):1877-87 (2015)), AAV3-ST (Vercauteren et al. Mol Ther. 24 (6):1042-1049 (2016)), Anc80 (Zinn E et al., Cell Rep. 12 (6):1056-68 (2015)) and any other AAV serotype now known or later discovered.

In a specific embodiment, the AAV viral particle comprises capsid proteins from a serotype selected from the group consisting of an AAV1, AAV3B, an AAV5, an AAV7, an AAV8, and an AAV9 which are more suitable for delivery to the liver cells (Nathwani et al. Blood 109: 1414-1421 (2007); Kitajima et al. Atherosclerosis 186:65-73 (2006)).

In a particular embodiment, the AAV viral particle comprises capsid proteins from Anc80, a predicted ancestor of viral AAVs serotypes 1, 2, 8, and 9 that behaves as a highly potent gene therapy vector for targeting liver, muscle and retina (Zinn E et al., Cell Rep. 12 (6):1056-68 (2015)). In a more particular embodiment, the viral particle comprises the Anc80L65 VP3 capsid protein (Genbank accession number: KT235804).

Thus, in a further aspect, the present disclosure relates to a viral particle comprising a nucleic acid construct or expression vector of the disclosure and preferably comprising capsid proteins of adeno-associated virus such as capsid proteins are selected from the group consisting of: AAV3 type 3A, AAV3 type 3B, NP40, NP59, NP84, LK03, AAV3-ST, Anc80, AAV9 and AAV8 serotype, preferably AAV8 serotype.

In a particular embodiment, the viral particle comprises AAV vector genome comprised in recombinant baculovirus. Thus, a second recombinant baculovirus genome comprising

AAV rep and cap is used for producing AAV viral particle. In a particular embodiment, the rep and cap proteins are expressed from distinct baculovirus late promoters, preferably in inverse orientation. In a specific embodiment, that may be combined with the previous embodiments, the second baculovirus genome include a heterologous nucleic acid encoding the rep proteins, for example, rep proteins from AAV2 under the transcriptional control of the baculovirus polyhedron (P_{Ph}) promoter. In other embodiment, the second baculovirus genome includes a heterologous nucleic acid encoding the cap proteins under the transcriptional control of the p10 baculovirus promoter. Other modifications of the wild-type AAV sequences for proper expression in insect cells and/or to increase yield of VP and virion or to alter tropism or reduce antigenicity of the virion are also known in the art. By using helper baculoviral construct encoding the rep ORF (open reading frame) of an AAV serotype and cap ORF of a different serotype AAV, it is feasible packaging a vector flanked by ITRs of a given AAV serotype into virions assembled from structural capsid proteins of a different serotype. It is also possible by this same procedure to package mosaic, chimeric or targeted vectors.

Virus-glycan interactions are critical determinants of host cell invasion. In a particular embodiment, the AAV viral particle comprises capsid proteins comprising one or more amino acids substitutions, wherein the substitutions introduce a new glycan binding site into the AAV capsid protein. In a more particular embodiment, the amino acid substitutions are in amino acid 266, amino acids 463-475 and amino acids 499-502 in AAV2 or the corresponding amino acid positions in AAV1, AAV3, AAV4, AAV5, AAV6, AAV7, AAV 8, AAV9, AAV10 or any other AAV serotype, also included Anc80 and Anc80L65.

The introduced new glycan binding site can be a hexose binding site [e.g. a galactose (Gal), a mannose (Man), a glucose (Glu) or a fucose (fuc) binding site]; a sialic acid (Sia) binding site [e.g. a Sia residue such as is N-acetylneuraminic acid (NeuSAc) or N-Glycolylneuraminic acid (NeuSGc)]; or a disaccharide binding site, wherein the disaccharide is a sialic acid linked to galactose, for instance in the form of Sia(alpha2,3)Gal or Sia(alpha2,6)Gal. Detailed guidance to introduce a new binding site from an AAV serotype into a capsid protein of an AAV of another serotype is given on international patent publication WO2014144229 and in Shen et al. (J. Biol. Chem. 288 (40):28814-28823 (2013)). In a particular embodiment, the Gal binding site from AAV9 is introduced into the AAV2 VP3 backbone resulting in a dual glycan-binding AAV strain which is able to use both HS and Gal receptors for cell entry. Preferably, said dual glycan-binding AAV strain is AAV2G9. Shen et al. generated AAV2G9 by substituting amino acid residues directly involved and immediately flanking the Gal recognition site on the AAV9 VP3 capsid protein subunit onto corresponding residues on the AAV2 VP3 subunit coding region (AAV2 VP3 numbering Q464V, A467P, D469N, I470M, R471A, D472V, S474G, Y500F, and S501A).

In another embodiment, the viral particle for use according to the present disclosure may be an adenoviral particle, such as an Ad5 viral particle. As it is the case for AAV viral particle, capsid proteins of Ad viral particles can also be engineered to modify their tropism and cellular targeting properties, alternative adenoviral serotypes can also be employed.

In a particular embodiment, the viral particle according to the present invention comprises the expression vector comprising the minimal bile acid inducible promoter comprising or consisting of SEQ ID NO: 1, 6 or 7; a therapeutic transgene encoding BSEP, preferably of SEQ ID NO: 8, poly(A) sequence of SEQ ID NO: 10 and 5′ITR and 3′ITR of SEQ ID NO: 11 and 12, respectively.

In a particular embodiment, the viral particle according to the present invention comprises the expression vector comprising the minimal bile acid inducible promoter comprising or consisting of SEQ ID NO: 1, 6 or 7; a therapeutic transgene encoding MDR3, preferably of SEQ ID NO: 9, poly(A) sequence of SEQ ID NO: 10 and 5′ITR and 3′ITR of SEQ ID NO: 11 and 12, respectively.

In a particular embodiment, the viral particle according to the present invention comprises the expression vector comprising or consisting of SEQ ID NO: 13 or 14 or a sequence having at least 70, 75, 80, 85, 90, 95, 96, 97, 98, 99% of identity with SEQ ID NO: 13 or 14.

### A Process for Producing Viral Particles

Production of viral particles carrying the expression viral vector as disclosed above can be performed by means of conventional methods and protocols, which are selected taking into account the structural features chosen for the actual embodiment of expression vector and viral particle of the vector to be produced.

Briefly, viral particles can be produced in a host cell, more particularly in specific virus-producing cell (packaging cell), which is transfected with the nucleic acid construct or expression vector to be packaged, in the presence of a helper vector or virus or other DNA construct(s).

The term “packaging cells” as used herein, refers to a cell or cell line which may be transfected with a nucleic acid construct or expression vector of the invention and provides in trans all the missing functions which are required for the complete replication and packaging of a viral vector. Typically, the packaging cells express in a constitutive or inducible manner one or more of said missing viral functions. Said packaging cells can be adherent or suspension cells.

These packaging cells can be either producer cell lines expressing stably helper function for AAV production or cell lines transiently expressing part or totality of helper functions.

For example, said packaging cells may be eukaryotic cells such as mammalian cells, including simian, human, dog and rodent cells. Examples of human cells are PER.C6 cells (WO01/38362), MRC-5 (ATCC CCL-171), WI-38 (ATCC CCL-75), HEK-293 cells (ATCC CRL-1573), HEK293T cells (ATCC CRL-3216), and HeLa cells (ATCC CCL2).

Examples of non-human primate cells are Vero cells (ATCC CCL81), COS-1 cells (ATCC CRL-1650), COS-7 cells (ATCC CRL-1651) and fetal rhesus lung cells (ATCC CL-160). Examples of dog cells are MDCK cells (ATCC CCL-34). Examples of rodent cells are hamster cells, such as BHK21-F, HKCC cells, or CHO cells.

As an alternative to mammalian sources, the packaging cells for producing the viral particles may be derived from avian sources such as chicken, duck, goose, quail or pheasant. Examples of avian cell lines include avian embryonic stem cells (WO01/85938 and WO03/076601), immortalized duck retina cells (WO2005/042728), and avian embryonic stem cell derived cells, including chicken cells (WO2006/108846) or duck cells, such as EB66 cell line (WO2008/129058 & WO2008/142124).

In another embodiment, the cells can be any cells permissive for baculovirus infection and replication packaging cells. In a particular embodiment, said cells are insect cells, such as SF9 cells (ATCC CRL-1711), Sf21 cells (IPLB-Sf21), MG1 cells (BTI-TN-MG1) or High Five™ cells (BTI-TN-5B1-4).

Accordingly, in a particular embodiment, the packaging cell comprises:
a nucleic acid construct or expression vector according to the disclosure (e.g., the AAV expression vector according to the disclosure),
a nucleic acid construct, for example a plasmid, encoding AAV rep and/or cap genes which does not carry the ITR sequences; and/or
a nucleic acid construct, for example a plasmid or virus, comprising viral helper genes.

Typically, a process of producing viral particles comprises the following steps:
a) culturing a packaging cell comprising a nucleic acid construct or expression vector as described above in a culture medium; and
b) harvesting the viral particles from the cell culture supernatant and/or inside the cells.

Conventional methods can be used to produce AAV viral particles which consist on transient cell co-transfection with nucleic acid construct or expression vector (e.g. a plasmid) carrying the transgene of the invention; a nucleic acid construct (e.g., an AAV helper plasmid) that encodes rep and cap genes, but does not carry ITR sequences; and with a third nucleic acid construct (e.g., a plasmid) providing the adenoviral functions necessary for AAV replication. Viral genes necessary for AAV replication are referred herein as viral helper genes. Typically, said genes necessary for AAV replication are adenoviral helper genes, such as E1A, E1B, E2a, E4, or VA RNAs. Preferably, the adenoviral helper genes are of the Ad5 or Ad2 serotype.

Large-scale production of AAV particles according to the disclosure can also be carried out for example by infection of insect cells with a combination of recombinant baculoviruses (Urabe et al. Hum. Gene Ther. 13: 1935-1943 (2002)). SF9 cells are co-infected with two or three baculovirus vectors respectively expressing AAV rep, AAV cap and the AAV vector to be packaged. The recombinant baculovirus vectors will provide the viral helper gene functions required for virus replication and/or packaging. Smith et al. (Mol. The., 17 (11):1888-1896 (2009)) further describes a dual baculovirus expression system for large-scale production of AAV particles in insect cells.

Suitable culture media will be known to a person skilled in the art. The ingredients that compose such media may vary depending on the type of cell to be cultured. In addition to nutrient composition, osmolarity and pH are considered important parameters of culture media. The cell growth medium comprises a number of ingredients well known by the person skilled in the art, including amino acids, vitamins, organic and inorganic salts, sources of carbohydrate, lipids, trace elements (CuS04, FeS04, Fe(N03)3, ZnS04 . . . ), each ingredient being present in an amount which supports the cultivation of a cell in vitro (i.e., survival and growth of cells). Ingredients may also include different auxiliary substances, such as buffer substances (like sodium bicarbonate, Hepes, Tris . . . ), oxidation stabilizers, stabilizers to counteract mechanical stress, protease inhibitors, animal growth factors, plant hydrolyzates, anti-clumping agents, anti-foaming agents. Characteristics and compositions of the cell growth media vary depending on the particular cellular requirements. Examples of commercially available cell growth media are: MEM (Minimum Essential Medium), BME (Basal Medium Eagle) DMEM (Dulbecco's modified Eagle's Medium), Iscoves DMEM (Iscove's modification of Dulbecco's Medium), GMEM, RPMI 1640, Leibovitz L-15, McCoy's, Medium 199, Ham (Ham's Media) F10 and derivatives, Ham F12, DMEM/F12, etc.

Further guidance for the construction and production of viral vectors for use according to the disclosure can be found in Viral Vectors for Gene Therapy, Methods and Protocols. Series: Methods in Molecular Biology, Vol. 737. Merten and Al-Rubeai (Eds.); 2011 Humana Press (Springer); Gene Therapy. M. Giacca. 2010 Springer-Verlag; Heilbronn R. and Weger S. Viral Vectors for Gene Transfer: Current Status of Gene Therapeutics. In: Drug Delivery, Handbook of Experimental Pharmacology 197; M. Schafer-Korting (Ed.). 2010 Springer-Verlag; pp. 143-170; Adeno-Associated Virus: Methods and Protocols. R. O. Snyder and P. Moulllier (Eds). 2011 Humana Press (Springer); Bunning H. et al. Recent developments in adeno-associated virus technology. J. Gene Med. 10:717-733 (2008); Adenovirus: Methods and Protocols. M. Chillńon and A. Bosch (Eds.); Third Edition. 2014 Humana Press (Springer)

### Host cells

In another aspect, the invention relates to a host cell comprising a nucleic acid construct or an expression vector of the invention. More particularly, host cell according to the invention is a specific virus-producing cell, also named packaging cell which is transfected with the nucleic acid construct or expression vector according to the invention, in the presence of a helper vector or virus or other DNA constructs and provides in trans all the missing functions which are required for the complete replication and packaging of a viral particle. Said packaging cells can be adherent or suspension cells

For example, said packaging cells may be eukaryotic cells such as mammalian cells, including simian, human, dog and rodent cells. Examples of human cells are PER.C6 cells (WO01/38362), MRC-5 (ATCC CCL-171), WI-38 (ATCC CCL-75), HEK-293 cells (ATCC CRL-1573), HeLa cells (ATCC CCL2). Examples of non-human primate cells are Vero cells (ATCC CCL81), COS-1 cells (ATCC CRL-1650), COS-7 cells (ATCC CRL-1651) or fetal rhesus lung cells (ATCC CL-160). Examples of dog cells are MDCK cells (ATCC CCL-34). Examples of rodent cells are hamster cells, such as BHK21-F, HKCC cells, or CHO cells.

As an alternative to mammalian sources, the packaging cells for producing the viral particles may be derived from avian sources such as chicken, duck, goose, quail or pheasant. Examples of avian cell lines include avian embryonic stem cells (WO01/85938 and WO03/076601), immortalized duck retina cells (WO2005/042728), and avian embryonic stem cell derived cells, including chicken cells (WO2006/108846) or duck cells, such as EB66 cell line (WO2008/129058 & WO2008/142124).

In another embodiment, the cells can be any cells permissive for baculovirus infection and replication packaging cells. In a particular embodiment, said cells are insect cells, such as SF9 cells (ATCC CRL-1711), Sf21 cells (IPLB-Sf21), MG1 cells (BTI-TN-MG1) or High Five™ cells (BTI-TN-5B1-4).

Accordingly, in a particular embodiment, the host cell comprises:
a nucleic acid construct or expression vector comprising a transgene encoding BSEP according to the invention (e.g., the AAV vector according to the invention),
a nucleic acid construct, for example a plasmid, encoding AAV rep and/or cap genes which does not carry the ITR sequences; and/or
a nucleic acid construct, for example a plasmid or virus, comprising viral helper genes.

In another aspect, the invention relates to a host cell transduced with a viral particle of the invention and the term “host cell” as used herein refers to any cell line that is susceptible to infection by a virus of interest, and amenable to culture in vitro.

The host cell of the invention may be used for ex vivo gene therapy purposes. In such embodiments, the cells are transduced with the viral particle of the invention and subsequently transplanted to the patient or subject. Transplanted cells can have an autologous, allogenic or heterologous origin. For clinical use, cell isolation will generally be carried out under Good Manufacturing Practices (GMP) conditions. Before transplantation, cell quality and absence of microbial or other contaminants is typically checked and liver preconditioning, such as with radiation and/or an immunosuppressive treatment, may be carried out. Furthermore, the host cells may be transplanted together with growth factors to stimulate cell proliferation and/or differentiation, such as Hepatocyte Growth Factor (HGF).

In a particular embodiment, the host cell is used for ex vivo gene therapy into the liver. Preferably, said cells are eukaryotic cells such as mammalian cells, these include, but are not limited to, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like. A person skilled in the art will choose the more appropriate cells according to the patient or subject to be transplanted.

Said host cell may be a cell with self-renewal and pluripotency properties, such as stem cells or induced pluripotent stem cells. Stem cells are preferably mesenchymal stem cells (MSCs). MSCs are capable of differentiating into at least one the following types: an osteoblast, a chondrocyte, an adipocyte, or a myocyte and may be isolated from any type of tissue. Generally, MSCs will be isolated from bone marrow, adipose tissue, umbilical cord, or peripheral blood. Methods for obtaining thereof are well known to a person skilled in the art. Induced pluripotent stem cells (also known as iPS cells or iPSCs) are a type of pluripotent stem cell that can be generated directly from adult cells. Yamanaka et al. induced iPS cells by transferring the Oct3/4, Sox2, Klf4 and c-Myc genes into mouse and human fibroblasts, and forcing the cells to express the genes (WO 2007/069666). Thomson et al. subsequently produced human iPS cells using Nanog and Lin28 in place of Klf4 and c-Myc (WO 2008/118820).

Said host cells may also be hepatocytes. Hepatocyte transplantation procedures, including cell isolation and subsequent transplantation into a human or mice recipient is described for instance in Filippi and Dhawan, Ann NY Acad Sci. 1315:50-55 (2014); Yoshida et al., Gastroenterology 111:1654-1660 (1996); Irani et al. Mol. Ther. 3:3, 302-309 (2001); and Vogel et al. J Inherit Metab Dis 37:165-176 (2014). A method for ex vivo transduction of a viral particle into hepatocytes is described for instance in Merle et al., Scandinavian Journal of Gastroenterology 41:8, 974-982 (2006).

### Pharmaceutical Compositions

Another aspect of the present disclosure relates to a pharmaceutical composition comprising a nucleic acid construct, an expression vector, a viral particle or a host cell of the invention in combination with one or more pharmaceutical acceptable excipient, diluent or carrier.

As used herein, the term “pharmaceutically acceptable” means approved by a regulatory agency or recognized pharmacopeia such as European Pharmacopeia, for use in animals and/or humans. The term “excipient” refers to a diluent, adjuvant, carrier, or vehicle with which the therapeutic agent is administered.

Any suitable pharmaceutically acceptable carrier, diluent or excipient can be used in the preparation of a pharmaceutical composition (See e.g., Remington: The Science and Practice of Pharmacy, Alfonso R. Gennaro (Editor) Mack Publishing Company, April 1997). Pharmaceutical compositions are typically sterile and stable under the conditions of manufacture and storage. Pharmaceutical compositions may be formulated as solutions (e.g. saline, dextrose solution, or buffered solution, or other pharmaceutically acceptable sterile fluids), microemulsions, liposomes, or other ordered structure suitable to accommodate a high product concentration (e.g. microparticles or nanoparticles). The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition.

Prolonged absorption of the injectable compositions can be brought about by including in the composition an agent which delays absorption, for example, monostearate salts and gelatine. The product of the invention may be administered in a controlled release formulation, for example in a composition which includes a slow release polymer or other carriers that protect the product against rapid release, including implants and microencapsulated delivery systems. Biodegradable and biocompatible polymers may for example be used, such as ethylene vinyl acetate, polyanhydrides, polyglycolic acid, collagen, polyorthoesters, polylactic acid and polylactic/polyglycolic copolymers (PLG). Preferably, said pharmaceutical composition is formulated as a solution, more preferably as an optionally buffered saline solution. Supplementary active compounds can also be incorporated into the pharmaceutical compositions of the invention. Guidance on co-administration of additional therapeutics can for example be found in the Compendium of Pharmaceutical and Specialties (CPS) of the Canadian Pharmacists Association.

In one embodiment, the pharmaceutical composition is a parenteral pharmaceutical composition, including a composition suitable for intravenous, intraarterial, subcutaneous, intraperitoneal or intramuscular administration. These pharmaceutical compositions are exemplary only and do not limit the pharmaceutical compositions suitable for other parenteral and non-parenteral administration routes. The pharmaceutical compositions described herein can be packaged in single unit dosage or in multidosage forms.

### Therapeutic Uses

In a further aspect, the invention relates to a nucleic acid construct, expression vector, viral particle, host cell or pharmaceutical composition of the invention for use as a medicament in a subject in need thereof.

The term “subject” or “patient” as used herein, refers to mammals. Mammalian species that can benefit from the disclosed methods of treatment include, but are not limited to, humans, non-human primates such as apes, chimpanzees, monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like. In particular embodiment, said subject is neonate, an infant or, a child, more particularly a neonate or an infant. As used herein “neonate” refers to a baby who is less than 28 days and “infants” as used herein refers to a child who is between 29 days and 2 years.

Diseases or conditions that may be treated by administration of products described herein include, but are not limited to, liver-associated diseases, including alpha 1-antitrypsin deficiency, type I tyrosinemia, Progressive Familial Intrahepatic Cholestasis type 1, 2 and 3, Wilsons' disease, Crigler-Najjar syndrome type I, ornithine transcarbamylase (OTC) deficiency, familial hypercholesterolemia, coagulation disorders (e.g. hemophilia A and B, afibrogenemiahemophilia, von Willebrand's disease), viral infections of the liver (e.g. hepatitis virus infections, including hepatitis C virus), and liver cancers.

In a particular embodiment, the invention relates to a nucleic acid construct, expression vector, viral particle, host cell or pharmaceutical composition as described above for use in the treatment of a cholestatic liver diseases, in particular chronic cholestatic diseases.

Cholestatic liver disease are characterized by defective bile acid formation or transport from the liver to intestine that can manifest clinically with fatigue, pruritus and jaundice. Early biochemical evidence of cholestasis includes increases in serum alkaline phosphatase (ALP) and gamma-glutamyl transpeptidase (GGT), followed by onset of conjugated hyperbilirubinemia. Cholestasis that persists for longer than 3 to 6 months is generally considered to be chronic. Cholestatic disorders are broadly defined as intra- or extrahepatic. Intrahepatic cholestasis is caused by defects in bile canaliculi, hepatocellular function, or intrahepatic bile ducts. In contrast, causes of extrahepatic cholestasis affect the extrahepatic ducts, common hepatic duct, or common bile duct.

In a particular embodiment, the invention relates to a nucleic acid construct, expression vector, viral particle, host cell or pharmaceutical composition as described above for use in the treatment of an hereditary cholestasis. Hereditary cholestasis is a heterogeneous group of rare autosomal recessive liver disorders, which are characterized by intrahepatic cholestasis, pruritus, and jaundice and caused by defects in genes related to the secretion and transport of bile salts and lipids. Phenotypic manifestation is highly variable, ranging from progressive familial intrahepatic cholestasis (PFIC)—with onset in early infancy and progression to end-stage liver disease—to a milder intermittent mostly nonprogressive form known as benign recurrent intrahepatic cholestasis (BRIC). Cases have been reported of initially benign episodic cholestasis that subsequently transitions to a persistent progressive form of the disease. Therefore, BRIC and PFIC seem to represent two extremes of a continuous spectrum of phenotypes that comprise one disease. Thus far, five representatives of PFIC (named PFIC1-5) caused by pathogenic mutations present in both alleles of ATP8B1, ABCB11, ABCB4, TJP2, and NR1H4 have been described (Sticova et al., Canadian Journal of Gastroenterology and Hepatology, 2018).

In a more particular embodiment, the invention relates to a nucleic acid construct, expression vector, viral particle, host cell or pharmaceutical composition as described above for use in the treatment of a genetic cholestatic liver disease such as Progressive Familial Intrahepatic Cholestasis (PFIC), Benign recurrent, intermittent episodes of Cholestasis (BRIC), Alagille syndrome (AGS), Cystic Fibrosis (CF). In a more particular embodiment, said Progressive Familial Intrahepatic Cholestasis is progressive familial intrahepatic cholestasis type 1 (PFIC1), progressive familial intrahepatic cholestasis type 2 (PFIC2), or progressive familial intrahepatic cholestasis type 3 (PFIC3).

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. According to the present invention, examples of symptoms associated with cholestatic disease are hepatocyte death, decreased bile flow and accumulation of bile salts inside the hepatocyte and in blood, severe pruritus, permanent jaundice, evolution to portal hypertension, liver failure and cirrhosis. In other embodiments, this term refers to minimizing the spread or worsening of the disease resulting from the administration of one or more therapeutic agents to a subject with such a disease.

In a particular embodiment, cholestatic liver diseases is progressive familial intrahepatic cholestasis type 2 (PFIC2) or progressive familial intrahepatic cholestasis type 3 (PFIC3).

In a related aspect, the invention pertains to the use of a nucleic acid construct, expression vector, viral particle, host cell or pharmaceutical composition of the invention in the preparation of a medicament for use in the treatment of a liver disease, preferably for use in cholestatic liver diseases, more preferably progressive familial intrahepatic cholestasis such as PFIC2 or PFIC3.

In a further aspect, the invention relates to a method of treating and/or preventing a liver disease, preferably cholestatic liver diseases, more preferably progressive familial intrahepatic cholestasis such as PFIC2 or PFIC3, in a subject in need thereof that comprises administering to the subject a therapeutically effective amount of a nucleic acid construct, expression vector, viral particle, host cell or pharmaceutical composition of the invention.

In the context of the invention, an “effective amount” means a therapeutically effective amount.

As used herein a “therapeutically effective amount” refers to an amount effective, at dosages and for periods of time necessary to achieve the desired therapeutic result, such as amelioration or restoration of secretion of bile salts to bile. The therapeutically effective amount of the product of the invention, or pharmaceutical composition that comprises it may vary according to factors such as the disease state, age, sex, and weight of the individual, and the ability of the product or pharmaceutical composition to elicit a desired response in the individual. Dosage regimens may be adjusted to provide the optimum therapeutic response. A therapeutically effective amount is also typically one in which any toxic or detrimental effect of the product or pharmaceutical composition is outweighed by the therapeutically beneficial effects.

The treatment with a product of the invention may alleviate, ameliorate, or reduce the severity of one or more symptoms of cholestatic liver disease. For example, treatment may increase and/or restore secretion of bile salts to bile; decrease the amount of bile salts in liver and blood, decrease pruritus, decrease liver damage reducing transaminase levels in serum, and as a consequence may alleviate, ameliorate, or reduce the severity of the disease

The product of the invention will be typically included in a pharmaceutical composition or medicament, optionally in combination with a pharmaceutical carrier, diluent and/or adjuvant. Such composition or medicinal product comprises the product of the invention in an effective amount, sufficient to provide a desired therapeutic effect, and a pharmaceutically acceptable carrier or excipient.

In one embodiment the nucleic acid construct, expression vector, viral particle, host cell or pharmaceutical composition for its therapeutic use is administered to the subject or patient by a parenteral route, in particularly by intravenous, intraarterial, subcutaneous, intraperitoneal, or intramuscular route.

In one embodiment, the nucleic acid construct, expression vector, viral particle, host cell or pharmaceutical composition for therapeutic use is administered by interstitial route, i.e. by injection to or into the interstices of a tissue. The tissue target may be specific, for example the liver tissue, or it may be a combination of several tissues, for example the muscle and liver tissues. Exemplary tissue targets may include liver, skeletal muscle, heart muscle, adipose deposits, kidney, lung, vascular endothelium, epithelial and/or hematopoietic cells. In a preferred embodiment, it is administered by intrahepatic injection, i.e. injection into the interstitial space of hepatic tissue.

The amount of product of the invention that is administered to the subject or patient may vary depending on the particular circumstances of the individual subject or patient including, age, sex, and weight of the individual; the nature and stage of the disease, the aggressiveness of the disease; the route of administration; and/or concomitant medication that has been prescribed to the subject or patient. Dosage regimens may be adjusted to provide the optimum therapeutic response.

For any particular subject, specific dosage regimens may be adjusted over time according to the individual needs and the professional judgment of the person administering or supervising the administration of the compositions. Dosage ranges set forth herein are exemplary only and do not limit the dosage ranges that may be selected by medical practitioners.

In one embodiment, an AAV viral particle according to the invention can be administered to the subject or patient for the treatment of cholestatic liver disease in an amount or dose comprised within a range of 5×10¹¹ to 1×10¹⁵ vg/kg (vg: viral genomes; kg: subject's or patient's body weight).

### Kit

In another aspect, the invention further relates to a kit comprising a nucleic acid construct, expression vector, host cell, viral particle or pharmaceutical composition of the invention in one or more containers. The kit may include instructions or packaging materials that describe how to administer the nucleic acid construct, expression vector, viral particle, host cell or pharmaceutical compositions contained within the kit to a patient. Containers of the kit can be of any suitable material, e.g., glass, plastic, metal, etc., and of any suitable size, shape, or configuration. In certain embodiments, the kits may include one or more ampoules or syringes that contain the products of the invention in a suitable liquid or solution form.

The following examples are provided by way of illustration, and they are not intended to be limiting of the present invention. Furthermore, the present invention covers all possible combinations of particular and preferred embodiments described herein.

### EXAMPLES

### 1. Generation of AAV Vectors Having a Luciferase Gene Downstream of Bile Salt Inducible Promoters

The first aim was to generate bile salt inducible promoters of small size (<300 nucleotides) to be included into AAV vectors harboring large transgenes, such as BSEP or MDR3, to regulate their expression and at the same time allow efficient packaging of the cDNA (Dong J Y, et al. Hum Gene Ther. 1996 Nov. 10; 7 (17):2101-12). The inventors first selected from the literature a minimal version of human BSEP promoter (ihPr) which contains an inverted repeat (IR)-1 element (5*-GGGACA TTGATCCT-3*) (SEQ ID NO: 15) at positions −63/−50 (from transcription initiation) formed by two nuclear receptor half-sites organized as an inverted repeat separated by a single nucleotide (Ananthanarayanan M et al. J Biol Chem 276 (31):28857-65. (2001)). The IR-1 element has been shown in several studies to function as a binding site for farnesoid X receptor (FXR), a nuclear transcription factor than can activate expression of both B SEP and MDR3 genes when bound to bile acids. The ihPr comprises the last 145 nucleotides (nt) of human BSEP promoter followed by the first 86 nt of human BSEP mRNA 5′ untranslated region (UTR) (SEQ ID NO: 2, based on NCBI reference sequence: AF190696.1). The inventors also designed a similar minimal BSEP promoter using sequences of murine origin (imPr). In this case, imPr comprises the last 145 nt of mouse BSEP promoter followed by the first 77 nt of mouse BSEP mRNA 5′UTR (SEQ ID NO: 1, based on NCBI reference sequence: AY039785.1).

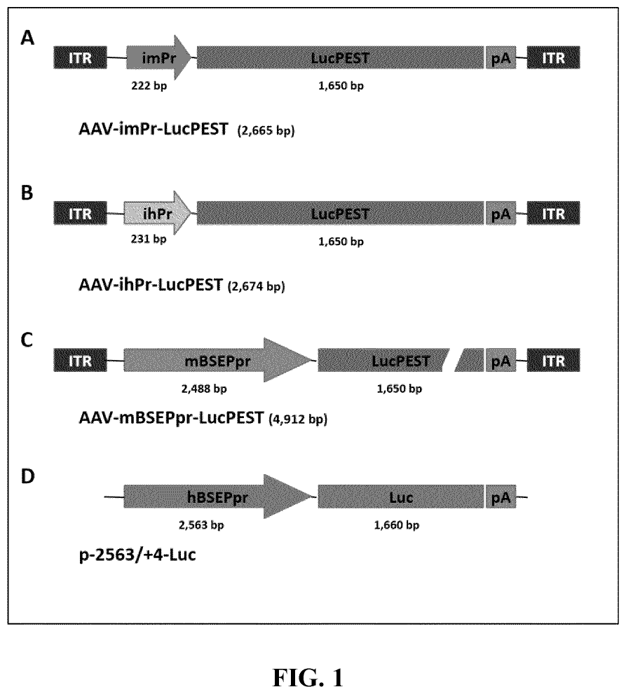

Both ihPr and imPr were ordered as synthetic sequences from GeneScript (Nanjing, China) cloned into pUC57 flanked by Mlu I and Nhe I restriction sites (pUC57-imPr & pUC57-ihPr). The inventors then proceeded to subclone these minimal promoters upstream of LucPEST (destabilized firefly luciferase) gene in AAV vector plasmids. For that purpose, the DNA fragment coding for each minimal promoter was extracted from pUC57 by digestion with Mlu I and Nhe I and subcloned into the same positions in plasmid pAAV-Al AT-LucPEST, substituting the A1AT promoter present in this vector with ihPr and imPr, and generating in this way the plasmids pAAV-ihPr-LucPEST (SEQ ID NO: 16) and pAAV-imPr-LucPEST (SEQ ID NO: 17), respectively. These vectors have an approximate size of 2.6 kb, which is within the packaging limits of AAV vectors. A diagram showing these vectors is presented in FIGS. 1A and 1B. The inventors also generated a control AAV vector having LucPEST downstream of a full-length mouse BSEP promoter of 2488 nt (pAAV-mBSEPpr-LucPEST) (SEQ ID NO: 18, FIG. 1C). The inventors ordered from GeneScript a synthetic sequence of this promoter (based on NCBI reference sequence AY039785.1) cloned into pUC57 flanked by Mlu I and Nhe I sites. The promoter was then extracted by digestion with Mlu I and Nhe I and subcloned into the same positions in plasmid pAAV-AlAT-LucPEST, substituting the A1AT promoter. An additional control vector containing the luciferase gene downstream of a full-length human BSEP promoter of 2563 nt (p-2563/+4-Luc) was kindly provided by Dr. Boyer (Yale University) (FIG. 1D).

### 2. Analysis of Luciferase Expression from Bile Salt Inducible Promoters in Human and Mouse Cell Lines

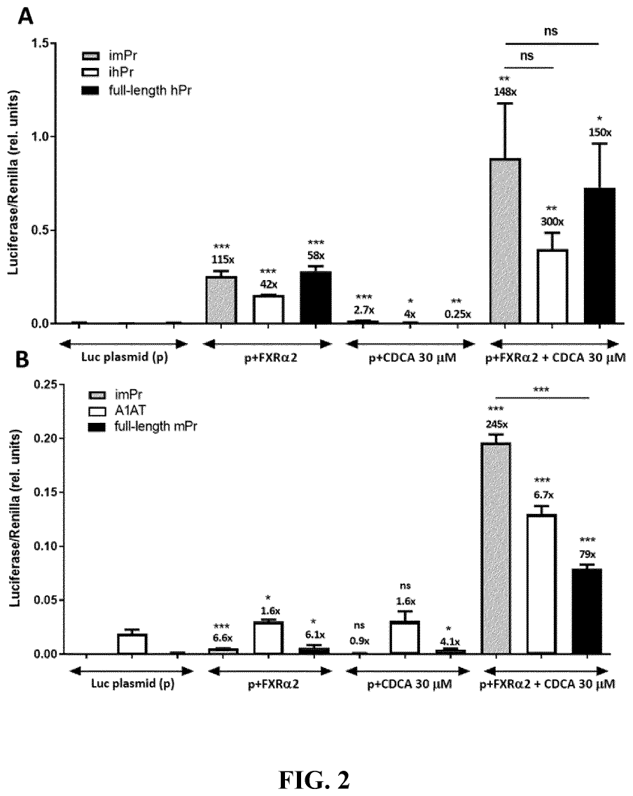

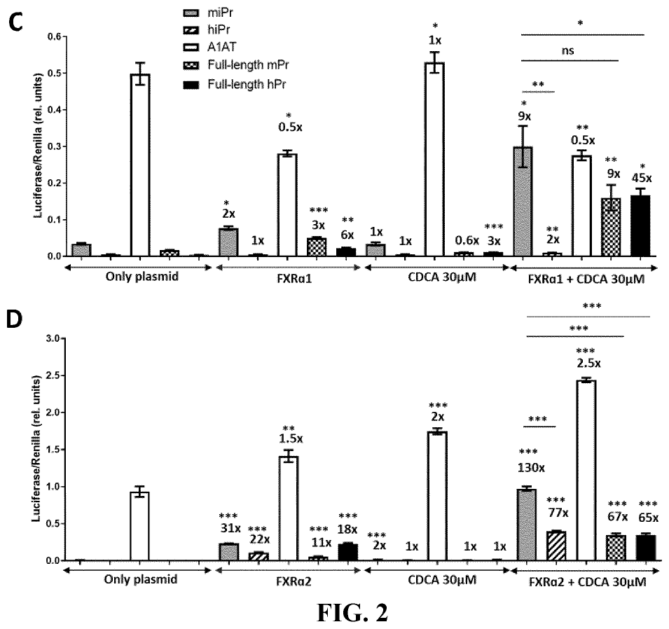

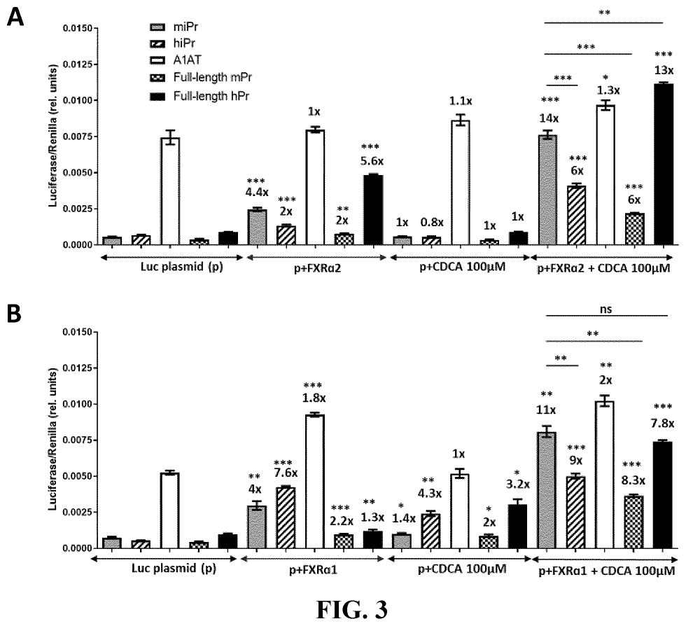

The inventors first tested whether luciferase expression could be induced by bile salts in hepatic cells from human (Huh-7 and HepG2) and mouse origin (Hepa1-6). For this purpose, they transfected confluent monolayers of these cells in 12-well plates with 0.5 μg pAAV-miPr-LucPEST or pAAV-hiPr-LucPEST plasmids, or with the same amount of control plasmids (pAAV-mBSEPpr-LucPEST, p-2563/+4-Luc and pAAV-A1AT-LucPEST), together with 0.5 μg of plasmids expressing either human FXRα1 (pcDNA3.1-hFXRα1, ordered from GenScript) or FXRα2 (pCMV-hFXRα2, ref NR1H4 cDNA Resource Center, Bloomsburg, PA), two isoforms of FXR that can exert different transcriptional regulation). The inventors included FXR-expressing plasmids in these experiments because previous studies performed with genetic constructs containing BSEP promoters have shown that hepatic cell lines do not express sufficient levels of FXR and had to be provided in trans to obtain optimal inducibility (Ananthanarayanan M et al. J Biol Chem. 276 (31):28857-65 (2001); Song X. et al. J Lipid Res. 54 (11):3030-44 (2013)). Furthermore, to normalize luciferase expression, and correct for differences in transfection efficiencies, the inventors also co-transfected cells with 0.2 μg of a plasmid expressing Renilla-luciferase from a constitutive promoter (pRL-CMV, Promega). All transfections were performed using lipofectamine 2000 (Thermo Fisher). For induction, 24 h after transfection, cells were incubated during 30 h in the presence of chenodeoxycholic acid (CDCA) at 30 μM (Huh-7 and HepG2) or at 100 μM (Hepa1-6), a bile acid that has been previously shown to activate BSEP expression. After induction, cells were collected and luciferase and Renilla expression were measured in cell extracts using an Orion L Microplate Luminometer. The inventors observed CDCA-mediated induction of luciferase expression when cells that received plasmids having BSEP promoters were also co-transfected with plasmids expressing FXR, with FXRα2 being more efficient in human cells (FIG. 2A-C), and both FXRα2 and FXRα1 being equally efficient in mouse cells (FIG. 3). Some degree of luciferase induction was also observed in cells receiving FXR plasmids without CDCA, but CDCA by itself did not increase luciferase expression. An increase in expression was also observed in cells that were transfected with pAAV-A1AT-LucPEST upon CDCA induction, but with a much lower fold induction compared to BSEP derived promoters.

Interestingly, in both human and mouse cell lines, imPr was able to provide higher inducibility and absolute expression levels compared to ihPr (FIGS. 2 and 3). This was an unexpected result, especially in human cells transactivated with human FXR isoforms, since the human version of the minimal promoter would be expected to be more active in these cells than the mouse minimal version. In fact, as expected, in Huh-7 cells the full-length human BSEP promoter provided higher induction and luciferase expression compared to full-length mouse BSEP promoter when FXRα2 was provided, and similar levels with FXRα1 (FIG. 2A-C). This was also the case in mouse Hepa1-6 cells regardless of the FXR isoform that was used (FIG. 3).

In Huh-7 cells transfected with FXRα2, imPr showed the highest level of inducibility (148-245-fold relative to uninduced control, data from two independent experiments shown in FIG. 2A and B), which was comparable to the inducibility observed with human endogenous full-length promoter (150-fold induction, FIG. 2A) and ihPr (300-fold, FIG. 2A). In addition, after CDCA-induction, imPr showed a total level of luciferase expression that was about two times higher than the one obtained with ihPr, and slightly higher than A1AT or human full-length BSEP promoters, highlighting the potency of this minimal mouse-derived promoter in human cells, especially taking into account that the plasmid with full-length human BSEP promoter expresses a non-destabilized luciferase (FIG. 2A).

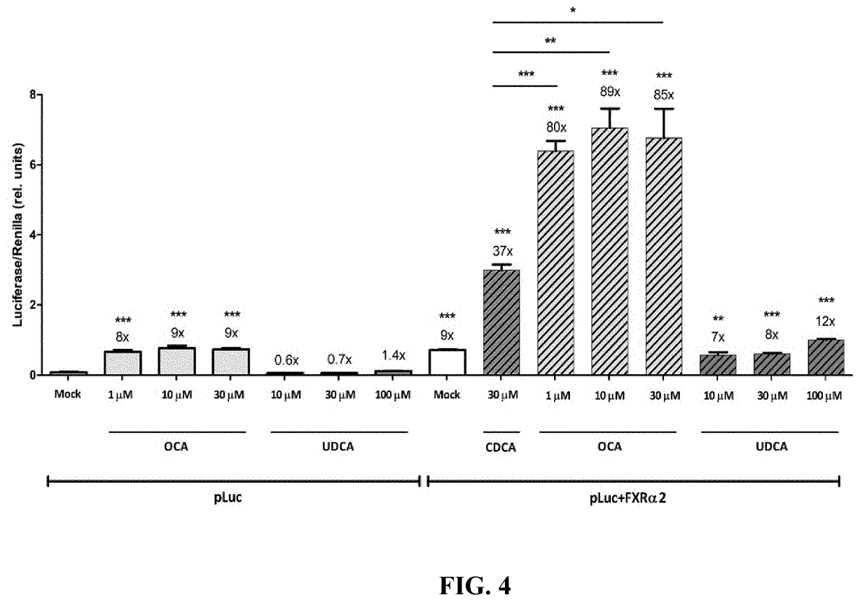

These results were recapitulated in a second human cell line (HepG2), where imPr showed the highest inducibility (130-fold) and expression levels when compared to other BSEP promoters (FIG. 2D). Interestingly, very similar results were observed in the mouse cell line Hepa1-6, suggesting that imPr could also be the best candidate to be tested in mice (FIG. 3). When experiments were performed with FXRα1, again imPr showed the highest inducibility and expression levels compared to other BSEP promoters, although in this case the fold change was lower (9 and 11 times in Huh-7 and Hepa1-6 respectively) (FIGS. 2C and 3B). Interestingly, while FXRα2 was able to provide higher expression and inducibility in human cells compared to FXRα1, in mouse cells both isoforms provided similar effects, as had been described in Song X. et al. J Lipid Res. 2013 November; 54 (11):3030-44. In addition, obeticholic acid (OCA) or ursodeoxycholic acid (UDCA), other bile acids (Bas) which have been described as selective FXR agonists able to activate the BSEP promoter and are used for treatment of several cholestatic diseases, including PFIC2 and PFIC3 (in the case of UDCA), were tested for inducibility of minimal BSEP promoters. Huh-7 cells were co-transfected with pAAV-imPr-LucPEST together with a plasmid that express human FXRα2. Twenty-four hours after transfection, cells were supplemented with different concentrations of OCA or UDCA or 30 μM CDCA as an effective BA-mediated induction control. The inventors observed that OCA and UDCA were also able to induce expression of luciferase under the control of imPr in human hepatic cells. Regarding the comparison with CDCA-mediated inducibility, the addition of UDCA showed lower absolute expression levels, while the addition of OCA reached higher levels even at a low concentration (1 μM) (FIG. 4). This result in human liver cells is interesting because it suggests that the use of vectors with inducible promoters in patients treated with UDCA or OCA could also increase expression of the therapeutic transgene.

### 3. Analysis of Luciferase Expression Induction in C57BL/6 Mice Receiving a Bile Salt Diet

The next step was to analyze whether imPr and ihPr could also be induced in vivo by bile salts. For that purpose, the inventors first generated AAV8 viral particles containing vectors AAV-imPr-LucPEST and AAV-ihPr-LucPEST, as well as AAV-A1AT-LucPEST as a non-inducible control, as described in Weber N. D. et al. 13; 10 (1):5694 (2019). Male and female four-week old C57BL/6 mice were intravenously injected with a viral dose of 3×10¹² VG/kg. Mice treated with each vector were divided into two groups: a first group received a normal diet throughout the whole length of the experiment and a second group was fed with chow containing 0.2% cholic acid (w/w) (CA) during three weeks followed by three weeks with normal chow, repeating this 6-week cycle three times. In all mice luciferase expression was measured weekly using a CCD luminometric camera (PhotonImager, Biospace lab) until sacrifice at 24 weeks post-inoculation.

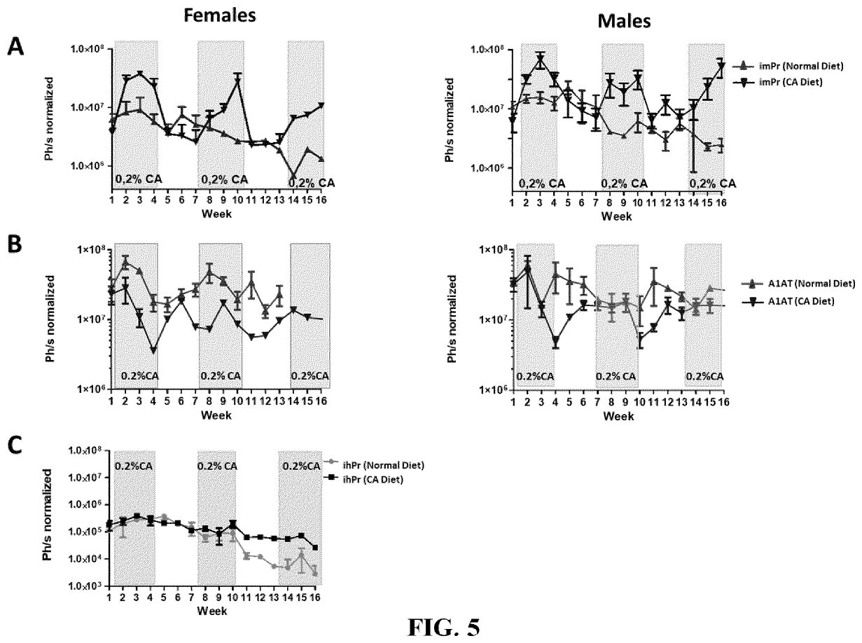

Mice inoculated with AAV-imPr-LucPEST showed a 5- to 10-fold increase in luciferase expression after administration of 0.2% CA diet in comparison with basal levels expressed with normal diet (FIG. 5A). In these groups no relevant differences between males and females were observed in either the level of induction or maximal luciferase expression, although this last parameter seemed to be slightly higher in males. Interestingly, reinduction of luciferase expression was successfully achieved during the three cycles of CA-supplemented diet. Moreover, with CA diet AAV-imPr-LucPEST provided similar, or even higher expression levels, than AAV-Al AT-LucPEST which showed high variability during the experiment regardless of diet and showed no diet-related induction of expression (FIG. 5B). Regarding AAV-ihPr-LucPEST, this vector showed very low expression levels with no luciferase induction in female mice receiving CA diet (it was not tested in male mice) (FIG. 5C).

### 4. Analysis of Luciferase Expression in Abcb4 KO Mice Compared to WT Mice

A mouse model for PFIC3 with an FVB background in which the Abcb4 gene was disrupted by elimination of its two first exons has been described in Smit, J. J. M., A. H. Schinkel, et al. Cell 75 (3): 451-462 (1993). Abcb4 KO mice completely lack expression of MDR2 (the mouse ortholog for human MDR3) and are unable to secrete phosphatidylcholine to the bile. The lack of MDR2 results in symptoms that reproduce most of the biomarkers and pathological signs of human PFIC3, including hepatosplenomegaly, liver fibrosis and high levels of serum transaminases and bile acids compared to FVB wild-type (WT) mice (Weber N. D. et al. Nat. Com. 10 (1):5694 (2019)). This last parameter makes them a good model for studying the inducibility of the minimal BSEP promoters developed in this work.

AAV8 vectors (AAV-imPr-LucPEST, AAV-ihPr-LucPEST, or AAV-AlAT-LucPEST) were intravenously injected into four-week old male and female FVB WT and Abcb4 KO mice at a dose of 3×10¹² VG/kg. In all mice luciferase expression was measured weekly using a CCD luminometric camera until sacrifice at 24 weeks post-inoculation.

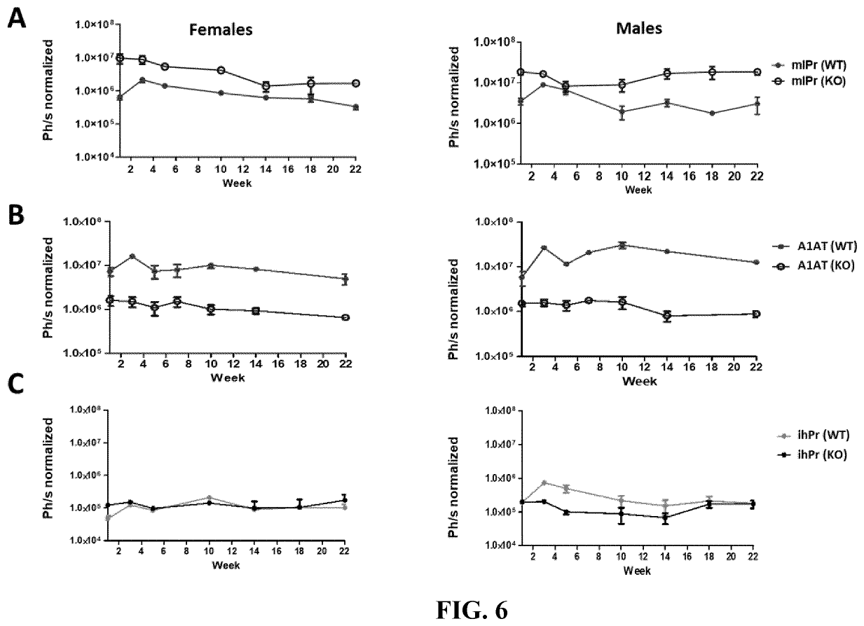

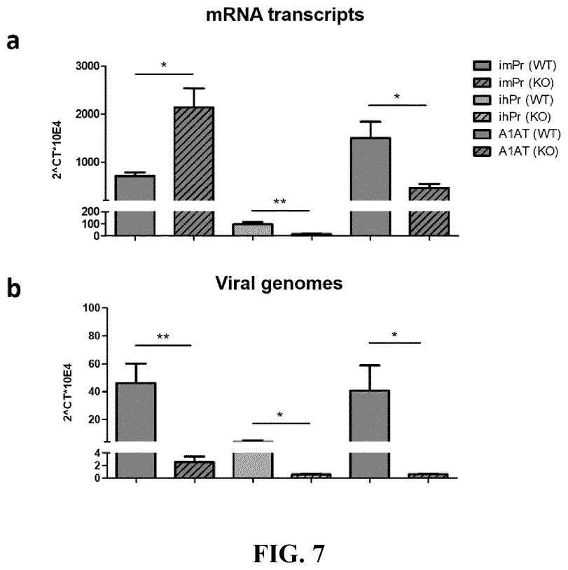

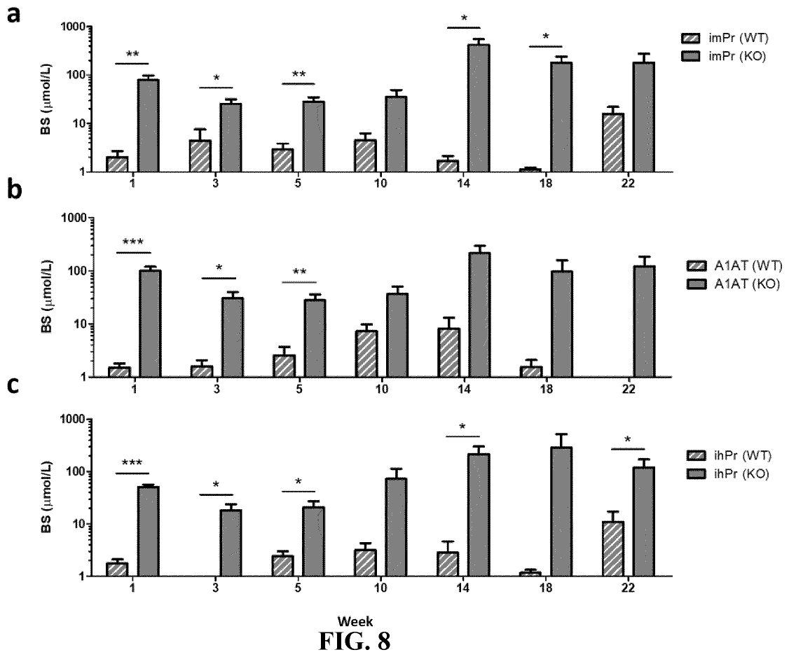

Interestingly, Abcb4 KO mice inoculated with AAV-imPr-LucPEST achieved a higher level of luciferase expression than WT mice inoculated with the same vector, suggesting a physiological induction of imPr without administering a CA-supplemented diet (FIG. 6A). Luciferase expression was slightly higher in males than females of both strains (WT and KO), which could be due to the fact that vector transduction is usually better in the liver of male- than female-mice. Moreover, AAV-imPr-LucPEST provided expression levels that were similar to the ones observed in WT mice receiving AAV-A1AT-LucPEST vector (FIG. 6B). However, this last vector expressed lower levels of luciferase in Abcb4 KO mice compared to WT mice, which was the opposite to what had been observed with AAV-imPr-LucPEST. This could be due to the fact that Abcb4 KO mice develop early liver damage that can decrease AAV transduction (Weber N. D. et al. Nat. Com. 10 (1):5694 (2019)). The inventors observed very similar results when they quantified liver LucPEST mRNA levels by RT-qPCR (FIG. 7a). However, Abcb4^{-/-} mice showed a significantly lower amount of AAV genomes than WT mice (FIG. 7b). The fact that despite this detrimental effect in transduction AAV-imPr-LucPEST showed higher expression in Abcb4 KO mice reinforces the idea that most likely the high bile salt levels present in these mice (Weber N. D. et al. Nat. Com. 10 (1):5694 (2019)) activate imPr-regulated transcription. In fact, Abcb4^{-/-} mice showed significantly higher levels of bile acid salts in serum when compared with WT mice (FIG. 8A-C). As observed in C57BL/6 mice, AAV-ihPr-LucPEST showed very low expression levels in vivo with no apparent differences between WT and Abcb4 KO mice (FIG. 6C).

### 5. Generation of AAV Vectors Having Therapeutic Genes (BSEP & MDR3) Downstream of Inducible BSEP Promoter imPr

The studies performed in cells and mice with AAV-imPr-LucPEST and AAV-ihPr-LucPEST vectors showed that imPr was able to mediate higher transgene expression and inducibility compared to ihPr. For that reason, the inventors selected imPr to be tested in AAV vectors expressing therapeutic genes with potential use for treatment of PFIC2 and PFIC3.

In the first case, the inventors generated an AAV vector expressing a codon-optimized version of ABCB11 cDNA (SEQ ID NO: 8) (coding for human BSEP) downstream of imPr (AAV-imPr-hBSEPco). To generate this vector, a DNA fragment containing the sequence of imPr was extracted from pAAV-imPr-LucPEST by Kpn I/Sac I digestion and subcloned into plasmid pAAV-A1AT-co-hBSEP (previously generated in the laboratory, patent EP18306458.3) substituting the A1AT promoter present in this vector (SEQ ID NO: 13).

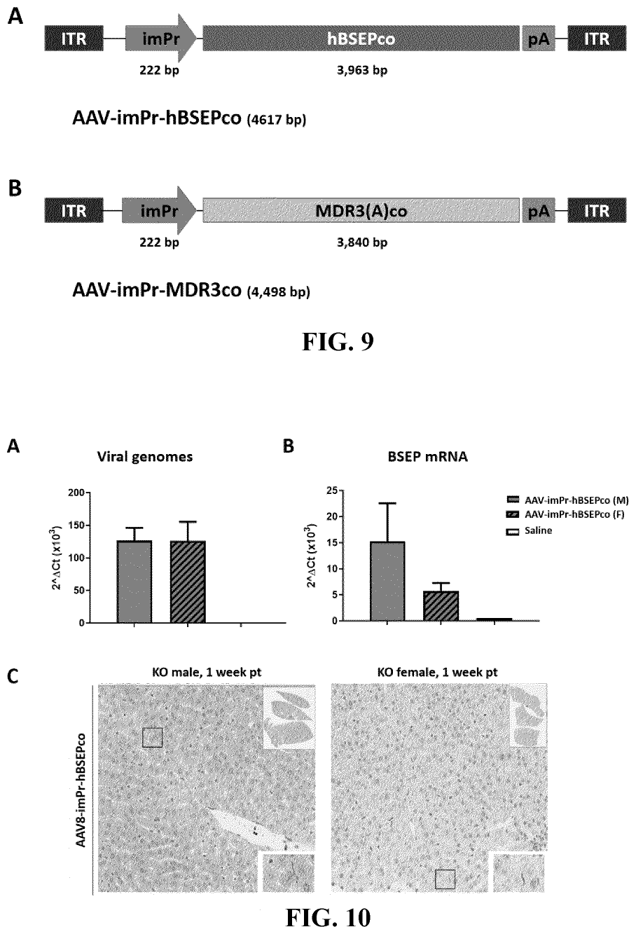

A similar strategy was used to generate an AAV vector expressing a human codon-optimized version of ABCB4 cDNA (SEQ ID NO: 9) (coding for MDR3 isoform A) downstream of imPr (AAV-imPr-MDR3co). In this case, the DNA fragment containing imPr sequence flanked by Kpn I and Sal I sites was subcloned into the same positions of plasmid pAAV-AlAT-MDR3co (previously generated in the laboratory, patent EP18306349) substituting the A1AT promoter present in this vector (SEQ ID NO: 14). AAV-imPr-hBSEPco and AAV-imPr-MDR3co have 4,617 and 4,498 bp, which is within the packaging limits of AAV vectors. A diagram showing the AAV-imPr therapeutic vectors is presented in FIG. 9.

### 6. Analysis of BSEP Expression and Therapeutic Efficacy of AAV-imPr-hBSEPco in Abcb11 KO Mice

### 6.1. Analysis of BSEP Expression In Vivo

For this study the inventors used Abcb11 KO C57BL/6 mice (Zhang et al. J Biol Chem. 2012 Jul. 13; 287 (29):24784-94) which have a dramatic decrease of biliary bile salts, leading to symptoms that recapitulate most of PFIC2-associated markers, such as elevated serum transaminases (ALT and AST) and bilirubin, hepatomegaly and liver fibrosis. However, in contrast to PFIC2 patients, these mice do not show an elevation of serum bile salts. AAV-imPr-hBSEPco vector or AAV-A1AT-hB SEPco, used as control, were produced using AAV8 capsid and used to inoculate four-week-old male and female Abcb11 KO C57BL/6 mice at a dose of 6×10¹³ VG/kg given intravenously. Animals were sacrificed 1 week after treatment and BSEP expression was analyzed in liver by RT-qPCR and immunohistochemistry (IHC).

AAV viral genomes were also analyzed by qPCR observing similar levels in males and females (FIG. 10A). BSEP mRNA was detected in all mice, being the levels higher in male mice (FIG. 10B). IHC showed BSEP diffuse expression in discrete pockets throughout the liver of mice receiving AAV-imPr-hBSEPco, with BSEP clearly located on the canalicular membrane of hepatocytes (FIG. 10C). These data indicate that AAV-imPr-hBSEPco can express BSEP in the liver, although the levels are lower than with a similar vector having the A1AT promoter. This result might be due to the fact thatAbcb11 KO C57BL/6 mice do not show elevation of bile salts in serum although they do develop other PFIC2 symptoms like elevation of transaminases and bilirubin, as well as liver fibrosis and hepatomegaly (patent EP18306458.3).

### 6.2. Analysis of Therapeutic Efficacy

In order to test the therapeutic efficacy of AAV-imPr-hBSEPco vector the inventors used the same Abcb11 KO C57BL/6 mouse strain (Zhang et al. J Biol Chem. 287 (29):24784-94 (2012)). The inventors treated four-week-old female Abcb11 KO mice via intraorbital injection with AAV-imPr-hBSEPco at a dose of 6×10¹³ VG/kg and monitored PFIC2 biomarkers in serum monthly during the following five months. The inventors used as controls mice treated with the same dose of AAV-A1AT-hBSEPco, as well as untreated Abcb11 KO and WT mice.

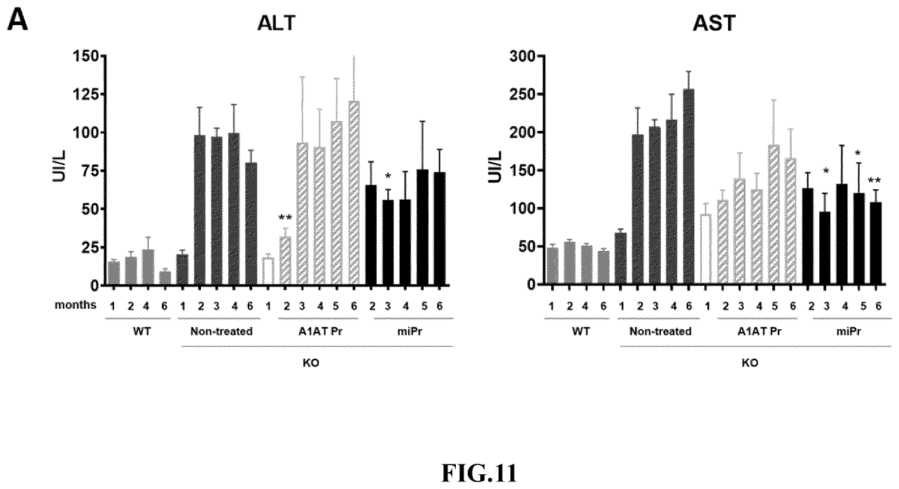

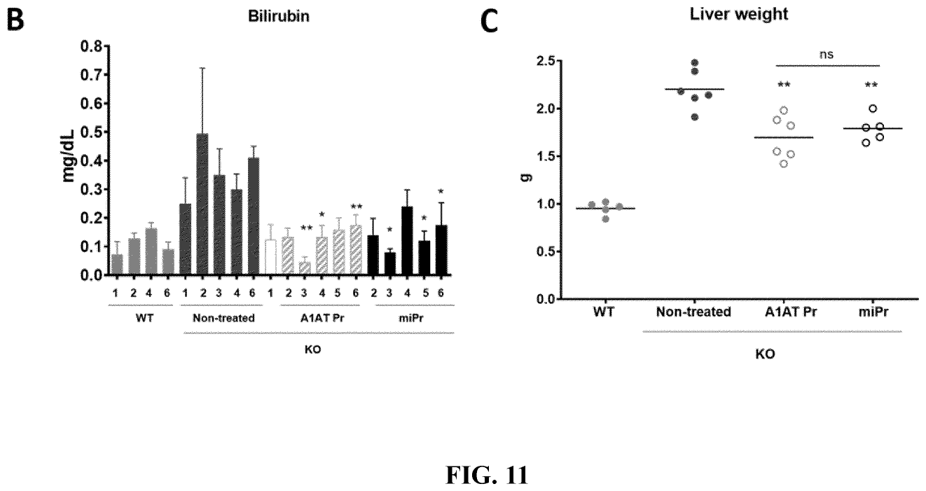

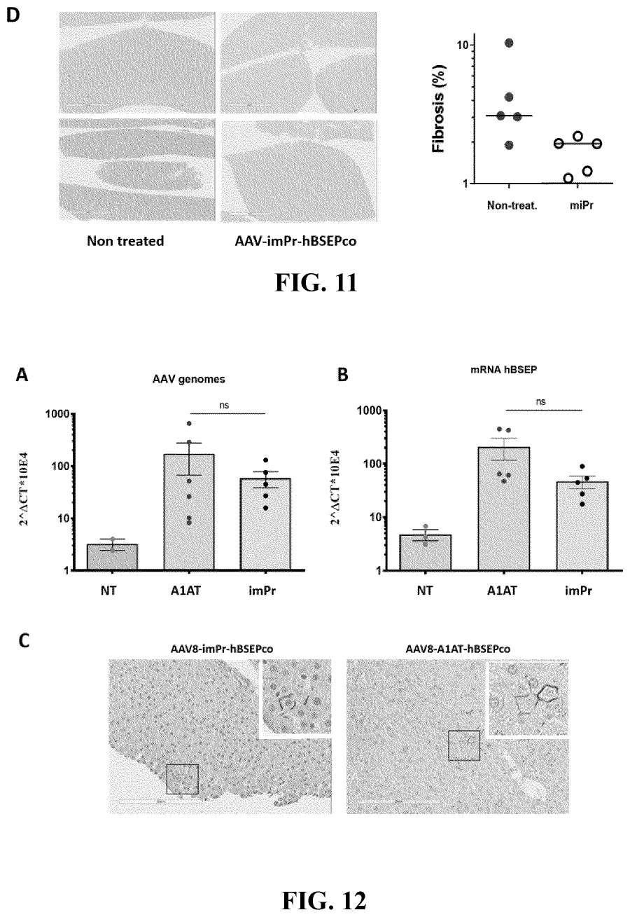

Mice treated with AAV-imPr-hBSEPco achieved a sustained therapeutic effect until the end of the study, as levels of aspartate aminotransferase (AST) and bilirubin in these mice were significantly reduced compared to untreated KO mice (FIG. 11A-B). This effect was even stronger than what was observed in mice that received control vector AAV-A1AT-hBSEPco. A reduction in alanine transaminase (ALT) was also observed, although it did not reach significance at all time points. When mice were sacrificed at five months post-treatment, a remarkable decrease in liver size was observed in mice that had received AAV-imPr-hBSEPco or AAV-A1AT-hBSEPco, compared to untreated KO mice, with no significant differences between the two treatment groups (FIG. 11C). In addition, the degree of liver fibrosis, (analyzed by picrosirius red staining), was lower in KO mice treated with AAV-imPr-hBSEPco in comparison with untreated mice (FIG. 11D). Finally, AAV genomes (FIG. 12A) and BSEP mRNA (FIG. 12B) were detected in liver of mice treated with AAV-miPr-hBSEPco and AAV-A1AT-hBSEPco, with no significant differences between the two groups. BSEP expression was also detected in biliary canaliculi by IHC, in both groups (FIG. 12C). Overall, these results indicate that the imPr can be used to control BSEP expression and reverse PFIC2 symptoms in a mouse model for this disease, despite the fact that these mice do not show an elevation of bile salts in serum. The high bile salt levels present in PFIC2 patients could lead to higher BSEP expression from the imPr vector, resulting in stronger therapeutic effects with physiological regulation.

### 7. Analysis of MDR3 Expression and Therapeutic Efficacy of AAV-imPr-MDR3co in Abcb4 KO Mice

### 7.1. Analysis of MDR3 Expression In Vivo

AAV-imPr-MDR3co vector was produced using AAV serotype 8 and used to inoculate two-week-old Abcb4 KO FVB male mice, which recapitulate PFIC3 symptoms (see section 4), with two different doses (1×10¹⁴ VG/kg and 1.5×10¹⁴ VG/kg) given intravenously. The AAV8 vector AAV-A1AT-MDR3co, used as control, was inoculated in two-week-old Abcb4 KO male mice at a dose of 1×10¹⁴ VG/kg. Animals were sacrificed one and two weeks after treatment and MDR3 expression was analyzed in liver by RT-qPCR and IHC. AAV viral genomes were also analyzed by qPCR.

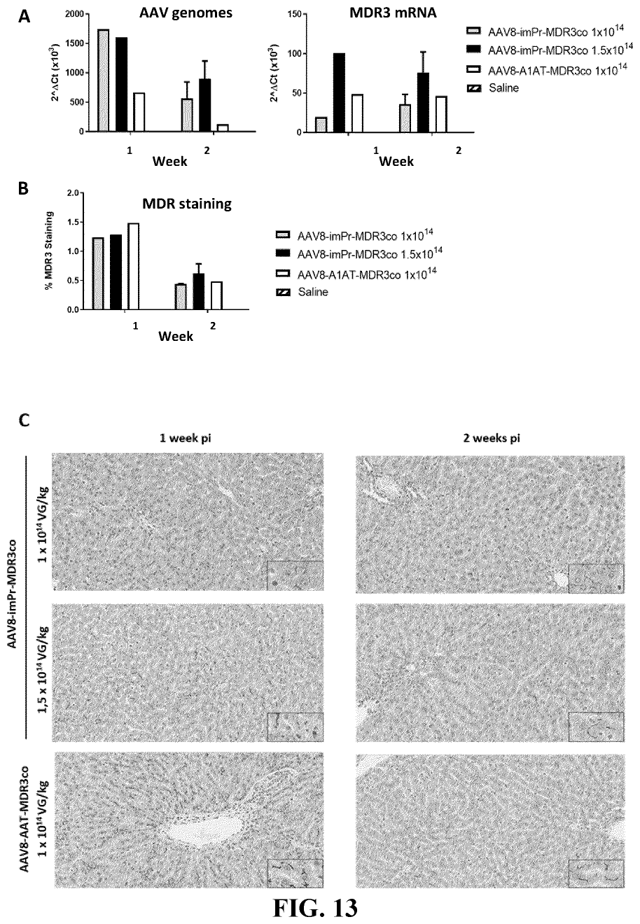

Quantification of viral genomes showed lower levels in mice receiving the control AAV-A1AT-MDR3co vector as compared to AAV-imPr-MDR3co. However, mRNA levels were comparable between the two vectors (FIG. 13A). IHC analysis showed MDR3 expression in the liver of mice receiving AAV-imPr-MDR3co, with a clear localization of the recombinant protein on the canalicular membrane of hepatocytes, similar to the one observed with the control vector having the A1AT promoter (FIG. 13B-C). These data indicate that AAV-imPr-MDR3co can efficiently express MDR3 in the liver. The level of MDR3 expression was determined as the percentage of positive tissue area with respect to the signal observed in WT mice (FIG. 13B). The inventors observed that at two weeks post-treatment, Abcb4 KO mice treated with AAV-imPr-MDR3co had 10% of the levels of expression observed in WT mice. This result is above the threshold of 2-3% MDR3 expression that the inventors have previously shown to be needed to reach a therapeutic effect (Weber N. D. et al. Nat. Com. 10 (1):5694 (2019)).

### 7.2. Analysis of Therapeutic Efficacy of AAV8-imPr-MDR3co

The inventors treated five-week-old male Abcb4 KO mice via intravenous injection with AAV-imPr-MDR3co at a dose of 1×10¹⁴ VG/kg and monitored PFIC3 biomarkers in serum monthly during the following two months. The inventors used as controls mice treated with the same dose of AAV-A1AT-MDR3(A)co, as well as untreated Abcb4 KO and WT mice.

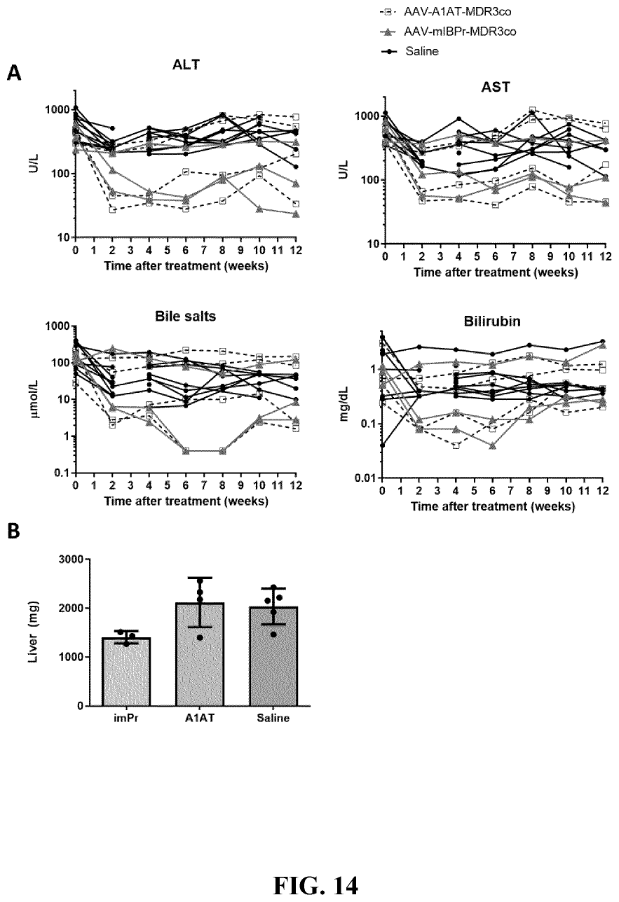

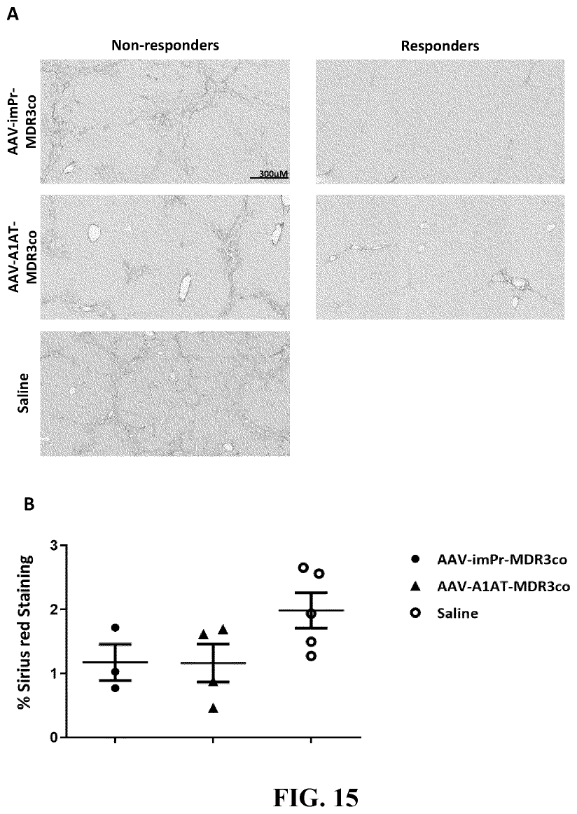

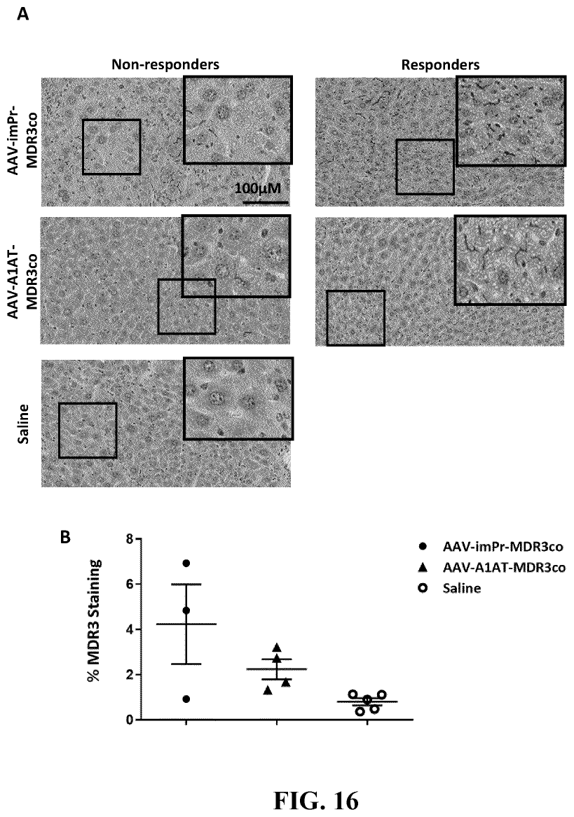

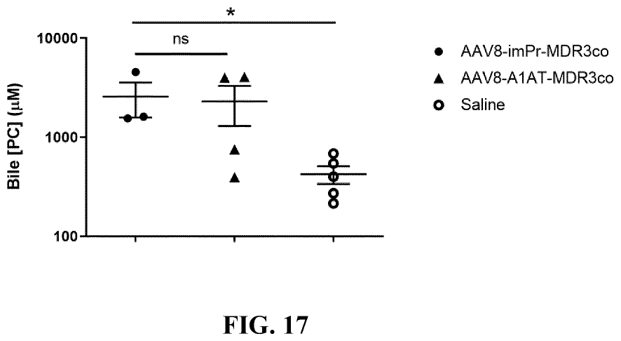

Two out three KO mice treated with AAV8-imPr-MDR3co achieved a sustained therapeutic effect until the end of the study, since levels of transaminases (ALT & AST), alkaline phosphatase and bile salts were reduced to levels similar to those observed in WT mice (FIG. 14A). This effect was similar to the one observed in mice that received control vector AAV-A1AT-MDR3(A)co, where two of the four treated mice achieved therapeutic effect. After three months, all mice were sacrificed in order to analyze liver and spleen size, liver histology, transduction efficiency, and transgene expression. Mice treated with the AAV-imPr-MDR3co vector showed a decrease in the degree of hepatomegaly compared to Abcb4 KO (FIG. 14B). Collagen levels indicative of the degree of liver fibrosis were analyzed from liver sections using picrosirius red staining. Responder mice treated with the therapeutic vectors showed a notable decrease of the degree of collagen staining in comparison with Abcb4 KO mice treated with saline. Non-responder mice showed a similar degree of fibrosis as the saline-treated mice (FIG. 15A). Quantification of fibrosis in all mice, performed using a FIJI V1.46b plugins (ImageJ) program, showed lower collagen levels in Abcb4 KO treated with AAV-imPr-MDR3co and AAV-A1AT-MDR3(A)co when compared to saline control KO mice (FIG. 15B). Next, MDR3 expression was analyzed by IHC. After three months of treatment, MDR3 expression was still detected abundantly in the bile canaliculi in responder Abcb4 KO mice treated with either therapeutic vector (FIG. 16A). However, MDR3 expression in non-responder Abcb4 KO mice was mild, exclusively localized in small foci. Notably, MDR3 staining in mice treated with the AAV-imPr-MDR3co vector showed a more intense recombinant protein staining pattern than mice treated with the AAV-A1AT-MDR3co (FIG. 16A). Interestingly, the percentage of MDR3 expression, determined as the percentage of positive tissue area with respect to the signal observed in WT mice using a FIJI V1.46b plugin (ImageJ) program, was higher in Abcb4 KO mice treated with AAV-imPr-MDR3co than in KO mice treated with AAV-A1AT-MDR3(A)co (FIG. 16B). Finally, phosphatidylcholine concentration in bile was increased in Abcb4 KO mice treated with either AAV-imPr-MDR3co or AAV-A1AT-MDR3(A)co vectors, compared to saline treated mice controls (FIG. 17). Overall, these results indicate that the imPr can be used to control MDR3 expression and reverse PFIC3 symptoms in a mouse model for this disease. These mice show a high elevation of bile salts in serum, which can probably increase expression from the imPr promoter via a physiological regulation. Interestingly, the fact that this promoter can also be activated by UDCA (FIG. 4), suggests that PFIC3 patients treated with AAV8-imPr-MDR3(A)co could have higher MDR3 expression when receiving UDCA, something that could lead to stronger therapeutic effects.

### 8. Design of Variants of imPr

In order to improve the inducibility and the level of expression of imPr promoter, the inventors have also designed and constructed variants of the inducible minimal promoter. For this purpose, three different strategies have been used:
1) The inventors designed and constructed variants of the imPr promoters that contain several repeats of the murine IR-1 element (TTAGGCCATTGACCTA, SEQ ID NO: 3), which acts as a binding element for the transcription factor FXR. These new promoters contain one (imPr+1xIR, SEQ ID NO: 6), three (imPr+3xIR, SEQ ID NO: 7) or five (imPr+5xIR, SEQ ID NO: 19) extra repeats of the IR-1 element cloned at the 5′end of imPr.
2) The inventors designed and constructed a variant of the imPr promoter that contains three repeats of the IR-1 element bound to the ER2 motif (TGGACT), which is necessary to achieve maximum FXR transactivation (Song X. et al. J Lipid Res. 54 (11):3030-44 (2013)). This variant of the imPr promoter contains three repetitions of the IR-1-ER2 element (TGGACTTTAGGCCATTGACCTA) (imPr+3xIR-ER2, SEQ ID NO: 20).
3) The inventors designed and constructed a slightly longer variant of the imPr that includes an upstream sequence corresponding to the so called LRH-1 element (TTTCTAAAGCT, SEQ ID NO: 5), which can regulate the expression of the BSEP promoter by acting as a modulator regulated by FXR13 (imPr+LRH-1, SEQ ID NO: 21).

These variants were ordered as synthetic sequences to Genscript and subcloned into the AAV-imPr-Luc-PEST plasmid replacing the imPr promoter using Kpn I and Sac I restriction sites, generating the following plasmids: pAAV-imPr+1xIR-LucPEST (SEQ ID NO: 22), pAAV-imPr+3xIR-LucPEST (SEQ ID NO: 23), pAAV-imPr+5xIR-LucPEST (SEQ ID NO: 24), pAAV-imPr+3xIR-ER2-LucPEST (SEQ ID NO: 25), and pAAV-imPr+LRH-1-LucPEST (SEQ ID NO: 26).

### 8. Analysis of Expression of the Variants of imPr

The inventors tested luciferase expression of plasmids containing the variants of imPr promoter in Huh-7 cells as described above. Briefly, the inventors transfected confluent cell monolayers in 12-well plates with 0.5 μg of each of the corresponding plasmids, using as control pAAV-miPr-LucPEST, together with 0.5 μg of pCMV-hFXRa2 using lipofectamine 2000. To normalize luciferase expression, the inventors co-transfected cells with 0.2 μg of a plasmid expressing Renilla-luciferase from a constitutive promoter (pRL-CMV). For induction, 24 h after transfection, cells were incubated during 30 h in the presence of 30 μM CDCA. After induction, cells were collected and luciferase and Renilla expression were measured in cell extracts as described.

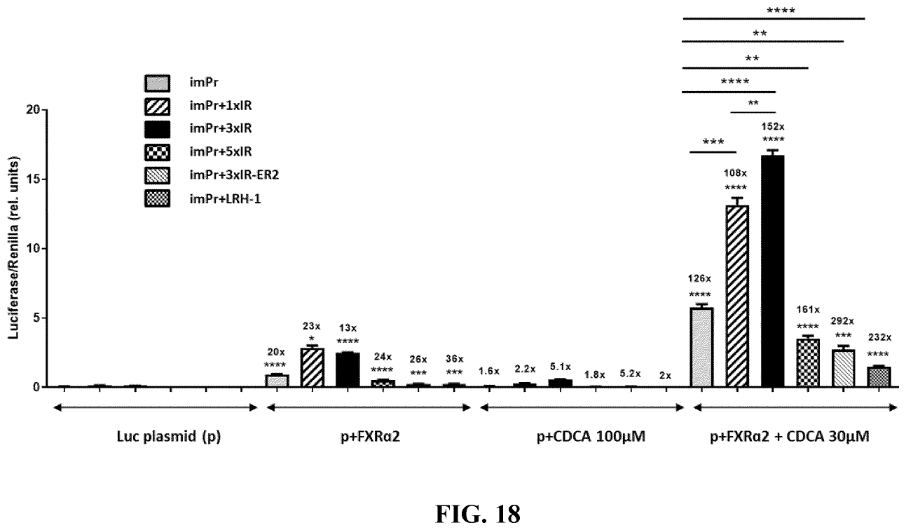

As observed in FIG. 18, vectors containing the imPr+1xIR and imPr+3xIR variants showed a significantly higher level of luciferase expression than the plasmid containing imPr promoter in cells co-transfected with pCMV-hFXRα2 and incubated with CDCA, indicating that the addition of one or three extra IR-1 sequences can enhance expression. In addition, imPr+3xIR promoter led to a significantly higher luciferase expression than imPr+1xIR. This effect was not observed with vectors containing imPr+5xIR, imPr+3xIR-ER2, and imPr+LRH-1 promoters.

### 9. An imPr with Three Extra IR-1 Repeats Shows Higher Inducible Expression in WT Mice Receiving a BA-enriched Diet

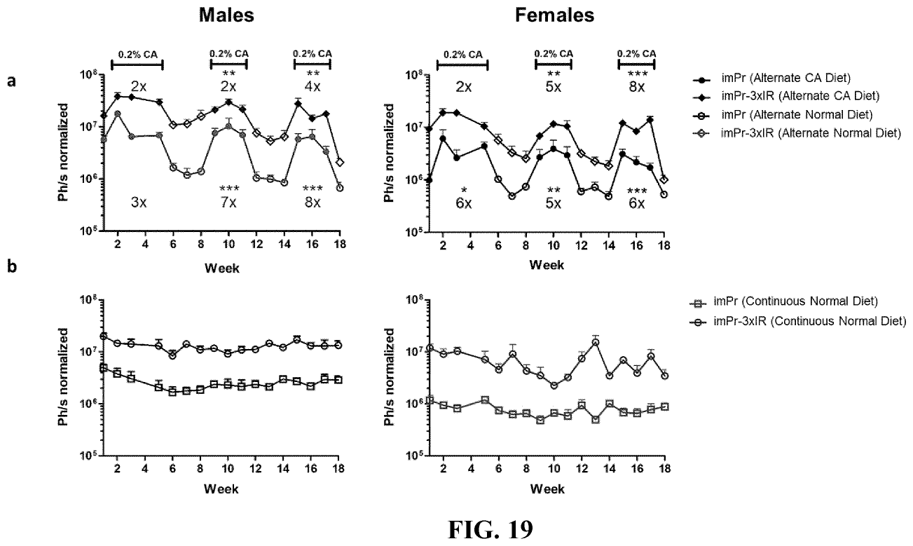

Next, four-week-old C57BL/6 WT male and female mice were administered intravenously with 3×10¹² VG/kg AAV8-imPr-LucPEST or AAV8-imPr-3xIR-LucPEST and submitted to cycles of CA diet supplementation as described before. Mice treated with AAV-imPr-3xIR-LucPEST showed a 2-8 fold increase in luciferase expression during each CA diet cycle compared to uninduced baseline levels (FIG. 19a). No relevant differences between males and females were observed in the degree of induction, but luciferase expression was again slightly higher in males. Upon induction, the expression levels obtained with AAV8-imPr-3xIR-LucPEST were higher than with AAV-imPr-LucPEST (2-9 fold increase) confirming the in vitro results. Baseline levels of mice on normal diet that received AAV-imPr-3xIR-LucPEST were also higher than for AAV-imPr-LucPEST (FIG. 19b).

### Sequences for Use in Practicing the Invention

Sequences for use in practicing the invention are described below:

imPr (minimal mouse BSEP promoter)
SEQ ID NO: 1
GGTTCCTGCTTTGAGTATGTTCGACCTTTCCTCTCATGTCACTGAACTGTGCTAGATCTGGAC
TTTAGGCCATTGACCTATAAGCAAATAGATAGTGTTCTTAAAAAAGCCTGATTTCTGTTCAAT
GCTTTATTACCATGAAAACTGAACTTGGAAAGGGGTGTACAACCCTGACTTTCCACAGTGGC
GTCTCTCGCTTCTCCTGGCTCCCTCAAATTCACA
ihPr (minimal human BSEP promoter)
SEQ ID NO: 2
TTCCCAAGCACACTCTGTGTTTGGGGTTATTGCTCTGAGTATGTTTCTCGTATGTCACTGAAC
TGTGCTTGGGCTGCCCTTAGGGACATTGATCCTTAGGCAAATAGATAATGTTCTTGAAAAAG
TTTGAATTCTGTTCAGTGCTTTAGAATGATGAAAACCGAGGTTGGAAAAGGTTGTGAAACCT
TTTAACTCTCCACAGTGGAGTCCATTATTTCCTCTGGCTTCCTC
Murine IR-1 element:
SEQ ID NO: 3
TTAGGCCATTGACCTA
Murine IR-1 element-ER2:
SEQ ID NO: 4
TGGACTTTAGGCCATTGACCTA
Liver receptor homolog 1 responsive element (LRHRE):
SEQ ID NO: 5
TTTCTAAAGCT
imPR+1 x IR
SEQ ID NO: 6
TTAGGCCATTGACCTAGGTTCCTGCTTTGAGTATGTTCGACCTTTCCTCTCATGTCACTGAAC
TGTGCTAGATCTGGACTTTAGGCCATTGACCTATAAGCAAATAGATAGTGTTCTTAAAAAAG
CCTGATTTCTGTTCAATGCTTTATTACCATGAAAACTGAACTTGGAAAGGGGTGTACAACCC
TGACTTTCCACAGTGGCGTCTCTCGCTTCTCCTGGCTCCCTCAAATTCACA
imPR + 3xIR
SEQ ID NO: 7
TTAGGCCATTGACCTATTAGGCCATTGACCTATTAGGCCATTGACCTAGGTTCCTGCTTTGAG
TATGTTCGACCTTTCCTCTCATGTCACTGAACTGTGCTAGATCTGGACTTTAGGCCATTGACC
TATAAGCAAATAGATAGTGTTCTTAAAAAAGCCTGATTTCTGTTCAATGCTTTATTACCATGA
AAACTGAACTTGGAAAGGGGTGTACAACCCTGACTTTCCACAGTGGCGTCTCTCGCTTCTCC
TGGCTCCCTCAAATTCACA
codon-optimized version of human ABCB11
SEQ ID NO: 8
ATGAGCGACTCCGTGATTCTGAGATCAATCAAAAAATTCGGCGAAGAAAATGACGGGTTCG
AGAGCGATAAATCCTATAATAATGACAAGAAGTCTAGGCTGCAGGACGAGAAGAAGGGCG
ATGGCGTGCGGGTGGGCTTCTTTCAGCTGTTCCGGTTCAGCAGCAGCACCGACATCTGGCTG
ATGTTTGTGGGCAGCCTGTGCGCCTTCCTGCACGGCATCGCACAGCCAGGCGTGCTGCTGAT
CTTTGGCACCATGACAGACGTGTTCATCGACTACGATGTGGAGCTGCAGGAGCTGCAGATCC
CTGGCAAAGCCTGCGTGAACAATACCATCGTGTGGACAAACAGCTCCCTGAACCAGAATAT
GACCAACGGCACACGCTGTGGCCTGCTGAATATCGAGTCTGAGATGATCAAGTTTGCCAGCT
ACTATGCAGGAATCGCAGTGGCCGTGCTGATCACCGGCTACATCCAGATTTGCTTCTGGGTC
ATCGCAGCAGCAAGGCAGATCCAGAAGATGAGAAAGTTCTATTTTCGGAGAATCATGCGGA
TGGAGATCGGCTGGTTTGACTGTAACTCTGTGGGCGAGCTGAATACAAGATTCAGCGACGAC
ATCAACAAGATCAATGACGCCATCGCCGATCAGATGGCCCTGTTTATCCAGCGGATGACCAG
CACAATCTGTGGCTTCCTGCTGGGCTTCTTTAGAGGCTGGAAGCTGACCCTGGTCATCATCA
GCGTGTCCCCACTGATCGGAATCGGAGCAGCAACAATCGGCCTGTCTGTGAGCAAGTTCACC
GACTACGAGCTGAAAGCCTACGCCAAGGCAGGAGTGGTGGCAGATGAAGTGATCAGCAGCA
TGAGGACCGTGGCAGCCTTTGGCGGAGAGAAGAGGGAGGTGGAGCGGTACGAGAAGAACC
TGGTGTTCGCCCAGCGGTGGGGCATCAGAAAGGGCATCGTGATGGGCTTCTTTACAGGCTTC
GTGTGGTGCCTGATCTTCCTGTGCTACGCCCTGGCCTTTTGGTATGGCTCCACCCTGGTGCTG
GACGAGGGAGAGTATACCCCTGGCACACTGGTGCAGATTTTCCTGAGCGTGATCGTGGGCGC
CCTGAACCTGGGAAATGCATCCCCATGCCTGGAAGCCTTCGCCACAGGAAGGGCAGCAGCC
ACCTCCATCTTCGAGACAATCGACCGCAAGCCTATCATCGACTGTATGTCTGAGGATGGCTA
CAAGCTGGACAGGATCAAGGGCGAGATCGAGTTTCACAATGTGACCTTCCACTATCCCAGCC
GCCCTGAGGTGAAGATCCTGAACGATCTGAATATGGTCATCAAGCCAGGAGAGATGACCGC
CCTGGTGGGACCCTCTGGAGCAGGCAAGAGCACCGCCCTGCAGCTGATCCAGCGGTTTTACG
ACCCTTGCGAGGGAATGGTGACCGTGGACGGACACGACATCAGGTCCCTGAACATCCAGTG
GCTGCGCGATCAGATCGGCATCGTGGAGCAGGAGCCAGTGCTGTTCTCTACCACAATCGCCG
AGAATATCAGATACGGCCGCGAGGATGCCACAATGGAGGACATCGTGCAGGCCGCCAAGGA
GGCCAACGCCTATAACTTCATCATGGATCTGCCCCAGCAGTTCGACACCCTGGTGGGAGAGG
GAGGAGGACAGATGTCCGGAGGCCAGAAGCAGAGAGTGGCCATCGCCAGAGCCCTGATCCG
CAACCCTAAGATCCTGCTGCTGGATATGGCCACAAGCGCCCTGGACAATGAGTCCGAGGCTA
TGGTGCAGGAGGTGCTGAGCAAGATCCAGCACGGCCACACCATCATCTCTGTGGCACACAG
GCTGAGCACAGTGAGAGCAGCCGACACCATCATCGGCTTTGAGCACGGCACAGCAGTGGAG
AGGGGCACCCACGAGGAGCTGCTGGAGAGGAAGGGCGTGTACTTCACCCTGGTGACACTGC
AGTCCCAGGGCAACCAGGCCCTGAATGAGGAGGACATCAAGGATGCCACAGAGGACGATAT
GCTGGCCCGGACCTTCAGCAGAGGCTCCTATCAGGATTCTCTGAGGGCCAGCATCAGGCAGC
GGAGCAAGTCTCAGCTGAGCTACCTGGTGCACGAGCCACCTCTGGCAGTGGTGGACCACAA
GTCCACCTATGAGGAGGATCGCAAGGACAAGGACATCCCAGTGCAGGAGGAGGTGGAGCCT
GCACCAGTGAGGCGCATCCTGAAGTTTTCCGCCCCAGAGTGGCCCTACATGCTGGTGGGATC
TGTGGGAGCAGCAGTGAACGGCACCGTGACACCACTGTATGCCTTCCTGTTTTCCCAGATCC
TGGGCACCTTCTCTATCCCCGACAAGGAGGAGCAGCGGTCCCAGATCAATGGCGTGTGCCTG
CTGTTTGTGGCTATGGGCTGCGTGAGCCTGTTTACACAGTTCCTGCAGGGCTACGCCTTCGCC
AAGAGCGGCGAGCTGCTGACCAAGCGGCTGAGAAAGTTCGGCTTTAGAGCCATGCTGGGCC
AGGACATCGCCTGGTTTGACGATCTGCGGAACAGCCCAGGCGCCCTGACCACAAGACTGGC
CACAGATGCATCTCAGGTGCAGGGAGCAGCAGGCAGCCAGATCGGCATGATCGTGAACTCC
TTCACCAATGTGACAGTGGCCATGATCATCGCCTTCAGCTTTTCCTGGAAGCTGAGCCTGGTC
ATCCTGTGCTTCTTCCCCTTTCTGGCCCTGAGCGGAGCAACCCAGACAAGGATGCTGACCGG
CTTCGCCTCCAGAGACAAGCAGGCCCTGGAGATGGTGGGCCAGATCACAAACGAGGCCCTG
AGCAATATCAGGACCGTGGCAGGAATCGGCAAGGAGCGGCGGTTCATCGAGGCCCTGGAGA
CAGAGCTGGAGAAGCCTTTCAAGACCGCCATCCAGAAGGCCAACATCTACGGCTTCTGCTTT
GCCTTCGCCCAGTGTATCATGTTCATCGCCAACTCTGCCAGCTACCGCTATGGCGGCTACCTG
ATCAGCAATGAGGGCCTGCACTTCAGCTACGTGTTCAGAGTGATCAGCGCCGTGGTGCTGTC
TGCCACAGCCCTGGGAAGGGCCTTCTCCTACACCCCATCTTATGCCAAGGCCAAGATCAGCG
CCGCCAGGTTCTTTCAGCTGCTGGACCGCCAGCCACCCATCAGCGTGTACAACACAGCCGGC
GAGAAGTGGGATAATTTCCAGGGCAAGATCGACTTTGTGGATTGCAAGTTCACCTATCCTAG
CAGACCAGACTCCCAGGTGCTGAATGGCCTGTCCGTGTCTATCAGCCCAGGCCAGACACTGG
CCTTTGTGGGCTCCTCTGGCTGTGGCAAGTCCACCTCTATCCAGCTGCTGGAGCGGTTCTATG
ACCCCGATCAGGGCAAAGTGATGATCGACGGCCACGATAGCAAGAAGGTGAACGTGCAGTT
TCTGAGATCCAATATCGGCATCGTGTCTCAGGAGCCTGTGCTGTTCGCCTGCTCCATCATGGA
TAACATCAAGTACGGCGACAATACAAAGGAGATCCCAATGGAGAGAGTGATCGCAGCAGCA
AAGCAGGCACAGCTGCACGATTTCGTGATGTCCCTGCCCGAGAAGTATGAGACAAACGTGG
GCTCTCAGGGCAGCCAGCTGTCCAGGGGCGAGAAGCAGAGGATCGCAATCGCCAGGGCCAT
CGTGCGCGATCCCAAGATCCTGCTGCTGGACGAGGCCACCAGCGCCCTGGATACAGAGTCC
GAGAAGACCGTGCAGGTGGCCCTGGACAAGGCCCGGGAGGGAAGAACATGTATCGTGATCG
CCCACAGACTGAGCACCATCCAGAATGCCGACATCATCGCCGTGATGGCCCAGGGCGTGGT
CATCGAGAAGGGCACCCACGAGGAACTGATGGCACAGAAAGGGGCTTACTACAAACTGGTC
ACAACAGGCTCACCTATCTCATAG
codon-optimized version of human ABCB4 cDNA
SEQ ID NO: 9
ATGGATCTGGAGGCCGCCAAGAACGGCACCGCCTGGAGACCCACAAGCGCCGAGGGCGACT
TCGAGCTGGGCATCAGCTCCAAGCAGAAGAGAAAGAAGACCAAGACAGTGAAGATGATCG
GCGTGCTGACACTGTTCAGGTACTCCGACTGGCAGGATAAGCTGTTTATGTCTCTGGGCACC
ATCATGGCAATCGCCCACGGCAGCGGCCTGCCTCTGATGATGATCGTGTTCGGCGAGATGAC
CGACAAGTTTGTGGATACAGCCGGCAATTTCTCCTTTCCCGTGAACTTCTCTCTGAGCCTGCT
GAACCCTGGCAAGATCCTGGAGGAGGAGATGACAAGATATGCCTACTATTACTCTGGCCTG
GGAGCAGGCGTGCTGGTGGCAGCATACATCCAGGTGAGCTTCTGGACCCTGGCAGCAGGCC
GGCAGATCAGAAAGATCAGGCAGAAGTTCTTTCACGCCATCCTGCGCCAGGAGATCGGCTG
GTTTGACATCAATGATACCACAGAGCTGAACACCCGGCTGACAGACGACATCTCTAAGATCA
GCGAGGGCATCGGCGATAAAGTGGGCATGTTCTTTCAGGCCGTGGCCACATTCTTTGCCGGC
TTCATCGTGGGCTTTATCAGGGGCTGGAAGCTGACCCTGGTCATCATGGCCATCTCTCCAATC
CTGGGCCTGAGCGCCGCCGTGTGGGCAAAGATCCTGTCCGCCTTCTCTGACAAGGAGCTGGC
CGCCTACGCCAAGGCAGGAGCAGTGGCAGAGGAGGCCCTGGGCGCCATCCGCACCGTGATC
GCCTTTGGCGGCCAGAATAAGGAGCTGGAGCGGTATCAGAAGCACCTGGAGAACGCCAAGG
AGATCGGCATCAAGAAGGCCATCTCCGCCAATATCTCTATGGGCATCGCCTTCCTGCTGATC
TATGCCAGCTACGCCCTGGCCTTTTGGTATGGCAGCACCCTGGTCATCAGCAAGGAGTACAC
CATCGGCAATGCCATGACAGTGTTCTTTAGCATCCTGATCGGAGCCTTCTCCGTGGGACAGG
CAGCACCATGCATCGACGCCTTCGCCAACGCCAGAGGCGCAGCCTACGTGATCTTTGACATC
ATCGATAACAATCCAAAGATCGACTCCTTTTCTGAGCGGGGCCACAAGCCCGATTCCATCAA
GGGCAATCTGGAGTTCAACGACGTGCACTTTAGCTATCCCTCCCGCGCCAATGTGAAGATCC
TGAAGGGCCTGAACCTGAAGGTGCAGTCCGGACAGACAGTGGCCCTGGTGGGCTCTAGCGG
ATGCGGCAAGTCTACCACAGTGCAGCTGATCCAGCGGCTGTATGACCCAGATGAGGGCACC
ATCAACATCGACGGCCAGGACATCCGCAACTTCAATGTGAACTACCTGCGGGAGATCATCG
GCGTGGTGTCTCAGGAGCCCGTGCTGTTTAGCACCACAATCGCCGAGAATATCTGTTATGGC
CGCGGCAACGTGACAATGGATGAGATCAAGAAGGCCGTGAAGGAGGCCAATGCCTACGAGT
TCATCATGAAGCTGCCCCAGAAGTTTGACACCCTGGTGGGAGAGAGAGGCGCCCAGCTGAG
CGGAGGCCAGAAGCAGAGGATCGCCATCGCCAGAGCCCTGGTGAGGAACCCTAAGATCCTG
CTGCTGGACGAGGCCACCTCCGCCCTGGATACAGAGTCTGAGGCAGAGGTGCAGGCCGCCC
TGGATAAGGCCCGCGAGGGACGGACCACAATCGTGATCGCCCACAGACTGAGCACAGTGAG
GAATGCCGACGTGATCGCCGGCTTCGAGGATGGCGTGATCGTGGAGCAGGGCAGCCACTCC
GAGCTGATGAAGAAGGAGGGCGTGTACTTCAAGCTGGTGAACATGCAGACCTCTGGCAGCC
AGATCCAGTCCGAGGAGTTTGAGCTGAATGACGAGAAGGCAGCAACAAGGATGGCACCTAA
CGGATGGAAGAGCAGACTGTTCAGGCACTCCACCCAGAAGAATCTGAAGAACTCTCAGATG
TGCCAGAAGAGCCTGGACGTGGAAACCGATGGACTGGAGGCAAATGTGCCACCCGTGAGCT
TCCTGAAGGTGCTGAAGCTGAACAAGACAGAGTGGCCATATTTTGTGGTGGGCACCGTGTGC
GCAATCGCAAATGGCGGCCTGCAGCCAGCCTTCAGCGTGATCTTTTCCGAGATCATCGCCAT
CTTCGGCCCTGGCGACGATGCCGTGAAGCAGCAGAAGTGTAACATCTTTTCCCTGATCTTCC
TGTTTCTGGGCATCATCTCTTTCTTTACCTTCTTTCTGCAGGGCTTCACATTTGGCAAGGCCGG
CGAGATCCTGACACGGAGACTGAGAAGCATGGCCTTCAAGGCCATGCTGAGGCAGGATATG
TCCTGGTTTGACGATCACAAGAACAGCACCGGCGCCCTGTCCACCAGACTGGCAACAGACG
CAGCACAGGTGCAGGGAGCAACCGGCACAAGGCTGGCCCTGATCGCCCAGAATATCGCCAA
CCTGGGCACAGGCATCATCATCTCTTTCATCTACGGCTGGCAGCTGACCCTGCTGCTGCTGGC
AGTGGTGCCAATCATCGCCGTGAGCGGCATCGTGGAGATGAAGCTGCTGGCCGGCAATGCC
AAGAGAGACAAGAAGGAGCTGGAGGCAGCAGGCAAGATCGCAACCGAGGCCATCGAGAAC
ATCCGCACCGTGGTGAGCCTGACACAGGAGCGGAAGTTCGAGTCCATGTATGTGGAGAAGC
TGTATGGCCCTTACCGCAATTCCGTGCAGAAGGCCCACATCTACGGCATCACCTTTTCCATCT
CTCAGGCTTTCATGTATTTTTCTTACGCCGGCTGCTTCCGGTTTGGCGCCTATCTGATCGTGA
ACGGCCACATGAGGTTCCGCGATGTGATCCTGGTGTTCTCTGCCATCGTGTTTGGAGCAGTG
GCCCTGGGACACGCCTCCTCTTTTGCCCCTGACTATGCAAAGGCAAAGCTGTCCGCCGCACA
CCTGTTCATGCTGTTTGAGAGACAGCCTCTGATCGATAGCTACTCCGAGGAGGGCCTGAAGC
CAGACAAGTTCGAGGGCAATATCACATTCAACGAGGTGGTGTTTAATTATCCAACCAGGGCC
AACGTGCCCGTGCTGCAGGGCCTGAGCCTGGAGGTGAAGAAGGGACAGACACTGGCCCTGG
TGGGCAGCTCCGGATGCGGCAAGTCCACCGTGGTGCAGCTGCTGGAGAGATTCTACGACCCT
CTGGCAGGCACCGTGCTGCTGGATGGACAGGAGGCCAAGAAGCTGAATGTGCAGTGGCTGA
GAGCCCAGCTGGGCATCGTGTCTCAGGAGCCAATCCTGTTCGATTGTAGCATCGCCGAGAAT
ATCGCCTACGGCGACAACTCTAGGGTGGTGAGCCAGGATGAGATCGTGAGCGCCGCAAAGG
CAGCAAACATCCACCCTTTTATCGAGACCCTGCCACACAAGTATGAGACACGCGTGGGCGAC
AAGGGCACCCAGCTGTCCGGAGGACAGAAGCAGAGGATCGCAATCGCCCGCGCCCTGATCA
GGCAGCCCCAGATCCTGCTGCTGGATGAAGCCACCTCCGCCCTGGACACAGAGTCTGAGAA
GGTGGTGCAGGAGGCCCTGGACAAAGCCCGCGAGGGACGGACATGTATTGTCATTGCTCAC
AGACTGAGCACCATCCAGAATGCCGACCTGATCGTGGTGTTCCAGAACGGCAGGGTGAAGG
AGCACGGCACACACCAGCAGCTGCTGGCCCAGAAGGGCATCTACTTTTCTATGGTGAGCGTG
CAGGCCGGCACCCAGAACCTGTAG
synthetic poly(A) signal sequence
SEQ ID NO: 10
AATAAAGACCTCTTATTTTCATTCATCAGGTGTGGTTGGTTTTTTTGTGTGGGGGC
5′ITR AAV2
SEQ ID NO: 11
CCTGCAGGCAGCTGCGCGCTCGCTCGCTCACTGAGGCCGCCCGGGCGTCGGGCGACCTTTGG
TCGCCCGGCCTCAGTGAGCGAGCGAGCGCGCAGAGAGGGAGTGGCCAACTCCATCACTAGG
GGTTCCT
3′ITR AAV2
SEQ ID NO: 12
AGGAACCCCTAGTGATGGAGTTGGCCACTCCCTCTCTGCGCGCTCGCTCGCTCACTGAGGCC
GGGCGACCAAAGGTCGCCCGACGCCCGGGCTTTGCCCGGGCGGCCTCAGTGAGCGAGCGAG
CGCGCAGCTGCCTGCAGG
AAV-imPr-hBSEPco
SEQ ID NO: 13
CCTGCAGGCAGCTGCGCGCTCGCTCGCTCACTGAGGCCGCCCGGGCGTCGGGCGACCTTTGG
TCGCCCGGCCTCAGTGAGCGAGCGAGCGCGCAGAGAGGGAGTGGCCAACTCCATCACTAGG
GGTTCCTGCGGCCGCGAATTCCATGGTACCGGTTCCTGCTTTGAGTATGTTCGACCTTTCCTC
TCATGTCACTGAACTGTGCTAGATCTGGACTTTAGGCCATTGACCTATAAGCAAATAGATAG
TGTTCTTAAAAAAGCCTGATTTCTGTTCAATGCTTTATTACCATGAAAACTGAACTTGGAAAG
GGGTGTACAACCCTGACTTTCCACAGTGGCGTCTCTCGCTTCTCCTGGCTCCCTCAAATTCAC
AgagctcGccgccaccATGAGCGACTCCGTGATTCTGAGATCAATCAAAAAATTCGGCGAAGAAAA
TGACGGGTTCGAGAGCGATAAATCCTATAATAATGACAAGAAGTCTAGGCTGCAGGACGAG
AAGAAGGGCGATGGCGTGCGGGTGGGCTTCTTTCAGCTGTTCCGGTTCAGCAGCAGCACCGA
CATCTGGCTGATGTTTGTGGGCAGCCTGTGCGCCTTCCTGCACGGCATCGCACAGCCAGGCG
TGCTGCTGATCTTTGGCACCATGACAGACGTGTTCATCGACTACGATGTGGAGCTGCAGGAG
CTGCAGATCCCTGGCAAAGCCTGCGTGAACAATACCATCGTGTGGACAAACAGCTCCCTGAA
CCAGAATATGACCAACGGCACACGCTGTGGCCTGCTGAATATCGAGTCTGAGATGATCAAGT
TTGCCAGCTACTATGCAGGAATCGCAGTGGCCGTGCTGATCACCGGCTACATCCAGATTTGC
TTCTGGGTCATCGCAGCAGCAAGGCAGATCCAGAAGATGAGAAAGTTCTATTTTCGGAGAAT
CATGCGGATGGAGATCGGCTGGTTTGACTGTAACTCTGTGGGCGAGCTGAATACAAGATTCA
GCGACGACATCAACAAGATCAATGACGCCATCGCCGATCAGATGGCCCTGTTTATCCAGCGG
ATGACCAGCACAATCTGTGGCTTCCTGCTGGGCTTCTTTAGAGGCTGGAAGCTGACCCTGGT
CATCATCAGCGTGTCCCCACTGATCGGAATCGGAGCAGCAACAATCGGCCTGTCTGTGAGCA
AGTTCACCGACTACGAGCTGAAAGCCTACGCCAAGGCAGGAGTGGTGGCAGATGAAGTGAT
CAGCAGCATGAGGACCGTGGCAGCCTTTGGCGGAGAGAAGAGGGAGGTGGAGCGGTACGA
GAAGAACCTGGTGTTCGCCCAGCGGTGGGGCATCAGAAAGGGCATCGTGATGGGCTTCTTTA
CAGGCTTCGTGTGGTGCCTGATCTTCCTGTGCTACGCCCTGGCCTTTTGGTATGGCTCCACCC
TGGTGCTGGACGAGGGAGAGTATACCCCTGGCACACTGGTGCAGATTTTCCTGAGCGTGATC
GTGGGCGCCCTGAACCTGGGAAATGCATCCCCATGCCTGGAAGCCTTCGCCACAGGAAGGG
CAGCAGCCACCTCCATCTTCGAGACAATCGACCGCAAGCCTATCATCGACTGTATGTCTGAG
GATGGCTACAAGCTGGACAGGATCAAGGGCGAGATCGAGTTTCACAATGTGACCTTCCACT
ATCCCAGCCGCCCTGAGGTGAAGATCCTGAACGATCTGAATATGGTCATCAAGCCAGGAGA
GATGACCGCCCTGGTGGGACCCTCTGGAGCAGGCAAGAGCACCGCCCTGCAGCTGATCCAG
CGGTTTTACGACCCTTGCGAGGGAATGGTGACCGTGGACGGACACGACATCAGGTCCCTGA
ACATCCAGTGGCTGCGCGATCAGATCGGCATCGTGGAGCAGGAGCCAGTGCTGTTCTCTACC
ACAATCGCCGAGAATATCAGATACGGCCGCGAGGATGCCACAATGGAGGACATCGTGCAGG
CCGCCAAGGAGGCCAACGCCTATAACTTCATCATGGATCTGCCCCAGCAGTTCGACACCCTG
GTGGGAGAGGGAGGAGGACAGATGTCCGGAGGCCAGAAGCAGAGAGTGGCCATCGCCAGA
GCCCTGATCCGCAACCCTAAGATCCTGCTGCTGGATATGGCCACAAGCGCCCTGGACAATGA
GTCCGAGGCTATGGTGCAGGAGGTGCTGAGCAAGATCCAGCACGGCCACACCATCATCTCT
GTGGCACACAGGCTGAGCACAGTGAGAGCAGCCGACACCATCATCGGCTTTGAGCACGGCA
CAGCAGTGGAGAGGGGCACCCACGAGGAGCTGCTGGAGAGGAAGGGCGTGTACTTCACCCT
GGTGACACTGCAGTCCCAGGGCAACCAGGCCCTGAATGAGGAGGACATCAAGGATGCCACA
GAGGACGATATGCTGGCCCGGACCTTCAGCAGAGGCTCCTATCAGGATTCTCTGAGGGCCAG
CATCAGGCAGCGGAGCAAGTCTCAGCTGAGCTACCTGGTGCACGAGCCACCTCTGGCAGTG
GTGGACCACAAGTCCACCTATGAGGAGGATCGCAAGGACAAGGACATCCCAGTGCAGGAGG
AGGTGGAGCCTGCACCAGTGAGGCGCATCCTGAAGTTTTCCGCCCCAGAGTGGCCCTACATG
CTGGTGGGATCTGTGGGAGCAGCAGTGAACGGCACCGTGACACCACTGTATGCCTTCCTGTT
TTCCCAGATCCTGGGCACCTTCTCTATCCCCGACAAGGAGGAGCAGCGGTCCCAGATCAATG
GCGTGTGCCTGCTGTTTGTGGCTATGGGCTGCGTGAGCCTGTTTACACAGTTCCTGCAGGGCT
ACGCCTTCGCCAAGAGCGGCGAGCTGCTGACCAAGCGGCTGAGAAAGTTCGGCTTTAGAGC
CATGCTGGGCCAGGACATCGCCTGGTTTGACGATCTGCGGAACAGCCCAGGCGCCCTGACCA
CAAGACTGGCCACAGATGCATCTCAGGTGCAGGGAGCAGCAGGCAGCCAGATCGGCATGAT
CGTGAACTCCTTCACCAATGTGACAGTGGCCATGATCATCGCCTTCAGCTTTTCCTGGAAGCT
GAGCCTGGTCATCCTGTGCTTCTTCCCCTTTCTGGCCCTGAGCGGAGCAACCCAGACAAGGA
TGCTGACCGGCTTCGCCTCCAGAGACAAGCAGGCCCTGGAGATGGTGGGCCAGATCACAAA
CGAGGCCCTGAGCAATATCAGGACCGTGGCAGGAATCGGCAAGGAGCGGCGGTTCATCGAG
GCCCTGGAGACAGAGCTGGAGAAGCCTTTCAAGACCGCCATCCAGAAGGCCAACATCTACG
GCTTCTGCTTTGCCTTCGCCCAGTGTATCATGTTCATCGCCAACTCTGCCAGCTACCGCTATG
GCGGCTACCTGATCAGCAATGAGGGCCTGCACTTCAGCTACGTGTTCAGAGTGATCAGCGCC
GTGGTGCTGTCTGCCACAGCCCTGGGAAGGGCCTTCTCCTACACCCCATCTTATGCCAAGGC
CAAGATCAGCGCCGCCAGGTTCTTTCAGCTGCTGGACCGCCAGCCACCCATCAGCGTGTACA
ACACAGCCGGCGAGAAGTGGGATAATTTCCAGGGCAAGATCGACTTTGTGGATTGCAAGTT
CACCTATCCTAGCAGACCAGACTCCCAGGTGCTGAATGGCCTGTCCGTGTCTATCAGCCCAG
GCCAGACACTGGCCTTTGTGGGCTCCTCTGGCTGTGGCAAGTCCACCTCTATCCAGCTGCTG
GAGCGGTTCTATGACCCCGATCAGGGCAAAGTGATGATCGACGGCCACGATAGCAAGAAGG
TGAACGTGCAGTTTCTGAGATCCAATATCGGCATCGTGTCTCAGGAGCCTGTGCTGTTCGCCT
GCTCCATCATGGATAACATCAAGTACGGCGACAATACAAAGGAGATCCCAATGGAGAGAGT
GATCGCAGCAGCAAAGCAGGCACAGCTGCACGATTTCGTGATGTCCCTGCCCGAGAAGTAT
GAGACAAACGTGGGCTCTCAGGGCAGCCAGCTGTCCAGGGGCGAGAAGCAGAGGATCGCA
ATCGCCAGGGCCATCGTGCGCGATCCCAAGATCCTGCTGCTGGACGAGGCCACCAGCGCCCT
GGATACAGAGTCCGAGAAGACCGTGCAGGTGGCCCTGGACAAGGCCCGGGAGGGAAGAAC
ATGTATCGTGATCGCCCACAGACTGAGCACCATCCAGAATGCCGACATCATCGCCGTGATGG
CCCAGGGCGTGGTCATCGAGAAGGGCACCCACGAGGAACTGATGGCACAGAAAGGGGCTTA
CTACAAACTGGTCACAACAGGCTCACCTATCTCATAGggatcCATATGATATCAATAAAGACCT
CTTATTTTCATTCATCAGGTGTGGTTGGTTTTTTTGTGTGGGGGCTCGAGATCTGAGGAACCC
CTAGTGATGGAGGCGGCCGCAGGAACCCCTAGTGATGGAGTTGGCCACTCCCTCTCTGCGCG
CTCGCTCGCTCACTGAGGCCGGGCGACCAAAGGTCGCCCGACGCCCGGGCTTTGCCCGGGCG
GCCTCAGTGAGCGAGCGAGCGCGCAGCTGCCTGCAG
AAV-imPr-MDR3co
SEQ ID NO: 14
CCTGCAGGCAGCTGCGCGCTCGCTCGCTCACTGAGGCCGCCCGGGCGTCGGGCGACCTTTGG
TCGCCCGGCCTCAGTGAGCGAGCGAGCGCGCAGAGAGGGAGTGGCCAACTCCATCACTAGG
GGTTCCTGCGGCCGCGAATTCCATGGTACCGGTTCCTGCTTTGAGTATGTTCGACCTTTCCTC
TCATGTCACTGAACTGTGCTAGATCTGGACTTTAGGCCATTGACCTATAAGCAAATAGATAG
TGTTCTTAAAAAAGCCTGATTTCTGTTCAATGCTTTATTACCATGAAAACTGAACTTGGAAAG
GGGTGTACAACCCTGACTTTCCACAGTGGCGTCTCTCGCTTCTCCTGGCTCCCTCAAATTCAC
AgagctcGTCGACGGATCCGCCGCCACCATGGATCTGGAGGCCGCCAAGAAcGGCACCGCcTGG
AGACCCACAAGCGCCGAGGGCGACTTCGAGCTGGGCATCAGCTCCAAGCAGAAGAGAAAG
AAGACCAAGACAGTGAAGATGATCGGCGTGCTGACACTGTTCAGGTACTCCGACTGGCAGG
ATAAGCTGTTTATGTCTCTGGGCACCATCATGGCAATCGCCCACGGCAGCGGCCTGCCTCTG
ATGATGATCGTGTTCGGCGAGATGACCGACAAGTTTGTGGATACAGCCGGCAATTTCTCCTT
TCCCGTGAACTTCTCTCTGAGCCTGCTGAACCCTGGCAAGATCCTGGAGGAGGAGATGACAA
GATATGCCTACTATTACTCTGGCCTGGGAGCAGGCGTGCTGGTGGCAGCATACATCCAGGTG
AGCTTCTGGACCCTGGCAGCAGGCCGGCAGATCAGAAAGATCAGGCAGAAGTTCTTTCACG
CCATCCTGCGCCAGGAGATCGGCTGGTTTGACATCAATGATACCACAGAGCTGAACACCCGG
CTGACAGACGACATCTCTAAGATCAGCGAGGGCATCGGCGATAAAGTGGGCATGTTCTTTCA
GGCCGTGGCCACATTCTTTGCCGGCTTCATCGTGGGCTTTATCAGGGGCTGGAAGCTGACCC
TGGTCATCATGGCCATCTCTCCAATCCTGGGCCTGAGCGCCGCCGTGTGGGCAAAGATCCTG
TCCGCCTTCTCTGACAAGGAGCTGGCCGCCTACGCCAAGGCAGGAGCAGTGGCAGAGGAGG
CCCTGGGCGCCATCCGCACCGTGATCGCCTTTGGCGGCCAGAATAAGGAGCTGGAGCGGTAT
CAGAAGCACCTGGAGAACGCCAAGGAGATCGGCATCAAGAAGGCCATCTCCGCCAATATCT
CTATGGGCATCGCCTTCCTGCTGATCTATGCCAGCTACGCCCTGGCCTTTTGGTATGGCAGCA
CCCTGGTCATCAGCAAGGAGTACACCATCGGCAATGCCATGACAGTGTTCTTTAGCATCCTG
ATCGGAGCCTTCTCCGTGGGACAGGCAGCACCATGCATCGACGCCTTCGCCAACGCCAGAG
GCGCAGCCTACGTGATCTTTGACATCATCGATAACAATCCAAAGATCGACTCCTTTTCTGAG
CGGGGCCACAAGCCCGATTCCATCAAGGGCAATCTGGAGTTCAACGACGTGCACTTTAGCTA
TCCCTCCCGCGCCAATGTGAAGATCCTGAAGGGCCTGAACCTGAAGGTGCAGTCCGGACAG
ACAGTGGCCCTGGTGGGCTCTAGCGGATGCGGCAAGTCTACCACAGTGCAGCTGATCCAGC
GGCTGTATGACCCAGATGAGGGCACCATCAACATCGACGGCCAGGACATCCGCAACTTCAA
TGTGAACTACCTGCGGGAGATCATCGGCGTGGTGTCTCAGGAGCCCGTGCTGTTTAGCACCA
CAATCGCCGAGAATATCTGTTATGGCCGCGGCAACGTGACAATGGATGAGATCAAGAAGGC
CGTGAAGGAGGCCAATGCCTACGAGTTCATCATGAAGCTGCCCCAGAAGTTTGACACCCTGG
TGGGAGAGAGAGGCGCCCAGCTGAGCGGAGGCCAGAAGCAGAGGATCGCCATCGCCAGAG
CCCTGGTGAGGAACCCTAAGATCCTGCTGCTGGACGAGGCCACCTCCGCCCTGGATACAGAG
TCTGAGGCAGAGGTGCAGGCCGCCCTGGATAAGGCCCGCGAGGGACGGACCACAATCGTGA
TCGCCCACAGACTGAGCACAGTGAGGAATGCCGACGTGATCGCCGGCTTCGAGGATGGCGT
GATCGTGGAGCAGGGCAGCCACTCCGAGCTGATGAAGAAGGAGGGCGTGTACTTCAAGCTG
GTGAACATGCAGACCTCTGGCAGCCAGATCCAGTCCGAGGAGTTTGAGCTGAATGACGAGA
AGGCAGCAACAAGGATGGCACCTAACGGATGGAAGAGCAGACTGTTCAGGCACTCCACCCA
GAAGAATCTGAAGAACTCTCAGATGTGCCAGAAGAGCCTGGACGTGGAAACCGATGGACTG
GAGGCAAATGTGCCACCCGTGAGCTTCCTGAAGGTGCTGAAGCTGAACAAGACAGAGTGGC
CATATTTTGTGGTGGGCACCGTGTGCGCAATCGCAAATGGCGGCCTGCAGCCAGCCTTCAGC
GTGATCTTTTCCGAGATCATCGCCATCTTCGGCCCTGGCGACGATGCCGTGAAGCAGCAGAA
GTGTAACATCTTTTCCCTGATCTTCCTGTTTCTGGGCATCATCTCTTTCTTTACCTTCTTTCTGC
AGGGCTTCACATTTGGCAAGGCCGGCGAGATCCTGACACGGAGACTGAGAAGCATGGCCTT
CAAGGCCATGCTGAGGCAGGATATGTCCTGGTTTGACGATCACAAGAACAGCACCGGCGCC
CTGTCCACCAGACTGGCAACAGACGCAGCACAGGTGCAGGGAGCAACCGGCACAAGGCTGG
CCCTGATCGCCCAGAATATCGCCAACCTGGGCACAGGCATCATCATCTCTTTCATCTACGGC
TGGCAGCTGACCCTGCTGCTGCTGGCAGTGGTGCCAATCATCGCCGTGAGCGGCATCGTGGA
GATGAAGCTGCTGGCCGGCAATGCCAAGAGAGACAAGAAGGAGCTGGAGGCAGCAGGCAA
GATCGCAACCGAGGCCATCGAGAACATCCGCACCGTGGTGAGCCTGACACAGGAGCGGAAG
TTCGAGTCCATGTATGTGGAGAAGCTGTATGGCCCTTACCGCAATTCCGTGCAGAAGGCCCA
CATCTACGGCATCACCTTTTCCATCTCTCAGGCTTTCATGTATTTTTCTTACGCCGGCTGCTTC
CGGTTTGGCGCCTATCTGATCGTGAACGGCCACATGAGGTTCCGCGATGTGATCCTGGTGTT
CTCTGCCATCGTGTTTGGAGCAGTGGCCCTGGGACACGCCTCCTCTTTTGCCCCTGACTATGC
AAAGGCAAAGCTGTCCGCCGCACACCTGTTCATGCTGTTTGAGAGACAGCCTCTGATCGATA
GCTACTCCGAGGAGGGCCTGAAGCCAGACAAGTTCGAGGGCAATATCACATTCAACGAGGT
GGTGTTTAATTATCCAACCAGGGCCAACGTGCCCGTGCTGCAGGGCCTGAGCCTGGAGGTGA
AGAAGGGACAGACACTGGCCCTGGTGGGCAGCTCCGGATGCGGCAAGTCCACCGTGGTGCA
GCTGCTGGAGAGATTCTACGACCCTCTGGCAGGCACCGTGCTGCTGGATGGACAGGAGGCC
AAGAAGCTGAATGTGCAGTGGCTGAGAGCCCAGCTGGGCATCGTGTCTCAGGAGCCAATCC
TGTTCGATTGTAGCATCGCCGAGAATATCGCCTACGGCGACAACTCTAGGGTGGTGAGCCAG
GATGAGATCGTGAGCGCCGCAAAGGCAGCAAACATCCACCCTTTTATCGAGACCCTGCCAC
ACAAGTATGAGACACGCGTGGGCGACAAGGGCACCCAGCTGTCCGGAGGACAGAAGCAGA
GGATCGCAATCGCCCGCGCCCTGATCAGGCAGCCCCAGATCCTGCTGCTGGATGAAGCCACC
TCCGCCCTGGACACAGAGTCTGAGAAGGTGGTGCAGGAGGCCCTGGACAAAGCCCGCGAGG
GACGGACATGTATTGTCATTGCTCACAGACTGAGCACCATCCAGAATGCCGACCTGATCGTG
GTGTTCCAGAACGGCAGGGTGAAGGAGCACGGCACACACCAGCAGCTGCTGGCCCAGAAGG
GCATCTACTTTTCTATGGTGAGCGTGCAGGCCGGCACCCAGAACCTGTAGCATATGATATCA
ATAAAGACCTCTTATTTTCATTCATCAGGTGTGGTTGGTTTTTTTGTGTGGGGGCTCGAGATC
TGAGGAACCCCTAGTGATGGAGGCGGCCGCAGGAACCCCTAGTGATGGAGTTGGCCACTCC
CTCTCTGCGCGCTCGCTCGCTCACTGAGGCCGGGCGACCAAAGGTCGCCCGACGCCCGGGCT
TTGCCCGGGCGGCCTCAGTGAGCGAGCGAGCGCGCAGCTGCCTGCAG
Human IR-1 element:
SEQ ID NO: 15
GGGACATTGATCCT
pAAV-ihPr-LucPEST
SEQ ID NO: 16
CCTGCAGGCAGCTGCGCGCTCGCTCGCTCACTGAGGCCGCCCGGGCGTCGGGCGAC
CTTTGGTCGCCCGGCCTCAGTGAGCGAGCGAGCGCGCAGAGAGGGAGTGGCCAAC
TCCATCACTAGGGGTTCCTGCGGCCGCACGCGTGGTACCTTCCCAAGCACACTCTGT
GTTTGGGGTTATTGCTCTGAGTATGTTTCTCGTATGTCACTGAACTGTGCTTGGGCT
GCCCTTAGGGACATTGATCCTTAGGCAAATAGATAATGTTCTTGAAAAAGTTTGAA
TTCTGTTCAGTGCTTTAGAATGATGAAAACCGAGGTTGGAAAAGGTTGTGAAACCT
TTTAACTCTCCACAGTGGAGTCCATTATTTCCTCTGGCTTCCTCGAGCTCGTCGACG
CTAGCTTGGCATTCCGGTACTGTTGGTAAAGCCACCATGGAAGACGCCAAAAACAT
AAAGAAAGGCCCGGCGCCATTCTATCCGCTGGAAGATGGAACCGCTGGAGAGCAA
CTGCATAAGGCTATGAAGAGATACGCCCTGGTTCCTGGAACAATTGCTTTTACAGA
TGCACATATCGAGGTGGACATCACTTACGCTGAGTACTTCGAAATGTCCGTTCGGTT
GGCAGAAGCTATGAAACGATATGGGCTGAATACAAATCACAGAATCGTCGTATGC
AGTGAAAACTCTCTTCAATTCTTTATGCCGGTGTTGGGCGCGTTATTTATCGGAGTT
GCAGTTGCGCCCGCGAACGACATTTATAATGAACGTGAATTGCTCAACAGTATGGG
CATTTCGCAGCCTACCGTGGTGTTCGTTTCCAAAAAGGGGTTGCAAAAAATTTTGA
ACGTGCAAAAAAAGCTCCCAATCATCCAAAAAATTATTATCATGGATTCTAAAACG
GATTACCAGGGATTTCAGTCGATGTACACGTTCGTCACATCTCATCTACCTCCCGGT
TTTAATGAATACGATTTTGTGCCAGAGTCCTTCGATAGGGACAAGACAATTGCACT
GATCATGAACTCCTCTGGATCTACTGGTCTGCCTAAAGGTGTCGCTCTGCCTCATAG
AACTGCCTGCGTGAGATTCTCGCATGCCAGAGATCCTATTTTTGGCAATCAAATCAT
TCCGGATACTGCGATTTTAAGTGTTGTTCCATTCCATCACGGTTTTGGAATGTTTACT
ACACTCGGATATTTGATATGTGGATTTCGAGTCGTCTTAATGTATAGATTTGAAGAA
GAGCTGTTTCTGAGGAGCCTTCAGGATTACAAGATTCAAAGTGCGCTGCTGGTGCC
AACCCTATTCTCCTTCTTCGCCAAAAGCACTCTGATTGACAAATACGATTTATCTAA
TTTACACGAAATTGCTTCTGGTGGCGCTCCCCTCTCTAAGGAAGTCGGGGAAGCGG
TTGCCAAGAGGTTCCATCTGCCAGGTATCAGGCAAGGATATGGGCTCACTGAGACT
ACATCAGCTATTCTGATTACACCCGAGGGGGATGATAAACCGGGCGCGGTCGGTAA
AGTTGTTCCATTTTTTGAAGCGAAGGTTGTGGATCTGGATACCGGGAAAACGCTGG
GCGTTAATCAAAGAGGCGAACTGTGTGTGAGAGGTCCTATGATTATGTCCGGTTAT
GTAAACAATCCGGAAGCGACCAACGCCTTGATTGACAAGGATGGATGGCTACATTC
TGGAGACATAGCTTACTGGGACGAAGACGAACACTTCTTCATCGTTGACCGCCTGA
AGTCTCTGATTAAGTACAAAGGCTATCAGGTGGCTCCCGCTGAATTGGAATCCATC
TTGCTCCAACACCCCAACATCTTCGACGCAGGTGTCGCAGGTCTTCCCGACGATGA
CGCCGGTGAACTTCCCGCCGCCGTTGTTGTTTTGGAGCACGGAAAGACGATGACGG
AAAAAGAGATCGTGGATTACGTCGCCAGTCAAGTAACAACCGCGAAAAAGTTGCG
CGGAGGAGTTGTGTTTGTGGACGAAGTACCGAAAGGTCTTACCGGAAAACTCGACG
CAAGAAAAATCAGAGAGATCCTCATAAAGGCCAAGAAGGGCGGAAAGATCGCCGT
GTCAAGCCATGGCTTCCCGCCGGCGGTGGCGGCGCAGGATGATGGCACGCTGCCCA
TGTCTTGTGCCCAGGAGAGCGGGATGGACCGTCACCCTGCAGCCTGTGCTTCTGCT
AGGATCAATGTGTAGGCGGCCAACAATGCGATCCGATGGCCGCGACTCTAGAGTCG
GGGCGGCCGGCCGCTTCGAGCAGACATGATAAGATACATTGATGAGTTTGGACAAA
CCACAACTAGAATGCAGTGAAAAAAATGCTTTATTTGTGAAATTTGTGATGCTATT
GCTTTATTTGTAACCATTATAAGCTGCAATAAACAAGTTAACAACAACAATTGCATT
CATTTTATGTTTCAGGTTCAGGGGGAGGTGTGGGAGGTTTTTTAAAGCAAGTAAAA
CCTCTACAAATGTGGTAAAATCGATAAGCCCGTGCGGACCGAGCGGCCGCAGGAA
CCCCTAGTGATGGAGTTGGCCACTCCCTCTCTGCGCGCTCGCTCGCTCACTGAGGCC
GGGCGACCAAAGGTCGCCCGACGCCCGGGCTTTGCCCGGGCGGCCTCAGTGAGCG
AGCGAGCGCGCAGCTGCCTGCAG
pAAV-imPr-LucPEST
SEQ ID NO: 17
CCTGCAGGCAGCTGCGCGCTCGCTCGCTCACTGAGGCCGCCCGGGCGTCGGGCGACCTTTGG
TCGCCCGGCCTCAGTGAGCGAGCGAGCGCGCAGAGAGGGAGTGGCCAACTCCATCACTAGG
GGTTCCTGCGGCCGCACGCGTGGTACCGGTTCCTGCTTTGAGTATGTTCGACCTTTCCTCTCA
TGTCACTGAACTGTGCTAGATCTGGACTTTAGGCCATTGACCTATAAGCAAATAGATAGTGT
TCTTAAAAAAGCCTGATTTCTGTTCAATGCTTTATTACCATGAAAACTGAACTTGGAAAGGG
GTGTACAACCCTGACTTTCCACAGTGGCGTCTCTCGCTTCTCCTGGCTCCCTCAAATTCACAG
AGCTCGTCGACGCTAGCTTGGCATTCCGGTACTGTTGGTAAAGCCACCATGGAAGACGCCAA
AAACATAAAGAAAGGCCCGGCGCCATTCTATCCGCTGGAAGATGGAACCGCTGGAGAGCAA
CTGCATAAGGCTATGAAGAGATACGCCCTGGTTCCTGGAACAATTGCTTTTACAGATGCACA
TATCGAGGTGGACATCACTTACGCTGAGTACTTCGAAATGTCCGTTCGGTTGGCAGAAGCTA
TGAAACGATATGGGCTGAATACAAATCACAGAATCGTCGTATGCAGTGAAAACTCTCTTCAA
TTCTTTATGCCGGTGTTGGGCGCGTTATTTATCGGAGTTGCAGTTGCGCCCGCGAACGACATT
TATAATGAACGTGAATTGCTCAACAGTATGGGCATTTCGCAGCCTACCGTGGTGTTCGTTTCC
AAAAAGGGGTTGCAAAAAATTTTGAACGTGCAAAAAAAGCTCCCAATCATCCAAAAAATTA
TTATCATGGATTCTAAAACGGATTACCAGGGATTTCAGTCGATGTACACGTTCGTCACATCTC
ATCTACCTCCCGGTTTTAATGAATACGATTTTGTGCCAGAGTCCTTCGATAGGGACAAGACA
ATTGCACTGATCATGAACTCCTCTGGATCTACTGGTCTGCCTAAAGGTGTCGCTCTGCCTCAT
AGAACTGCCTGCGTGAGATTCTCGCATGCCAGAGATCCTATTTTTGGCAATCAAATCATTCC
GGATACTGCGATTTTAAGTGTTGTTCCATTCCATCACGGTTTTGGAATGTTTACTACACTCGG
ATATTTGATATGTGGATTTCGAGTCGTCTTAATGTATAGATTTGAAGAAGAGCTGTTTCTGAG
GAGCCTTCAGGATTACAAGATTCAAAGTGCGCTGCTGGTGCCAACCCTATTCTCCTTCTTCGC
CAAAAGCACTCTGATTGACAAATACGATTTATCTAATTTACACGAAATTGCTTCTGGTGGCG
CTCCCCTCTCTAAGGAAGTCGGGGAAGCGGTTGCCAAGAGGTTCCATCTGCCAGGTATCAGG
CAAGGATATGGGCTCACTGAGACTACATCAGCTATTCTGATTACACCCGAGGGGGATGATAA
ACCGGGCGCGGTCGGTAAAGTTGTTCCATTTTTTGAAGCGAAGGTTGTGGATCTGGATACCG
GGAAAACGCTGGGCGTTAATCAAAGAGGCGAACTGTGTGTGAGAGGTCCTATGATTATGTC
CGGTTATGTAAACAATCCGGAAGCGACCAACGCCTTGATTGACAAGGATGGATGGCTACATT
CTGGAGACATAGCTTACTGGGACGAAGACGAACACTTCTTCATCGTTGACCGCCTGAAGTCT
CTGATTAAGTACAAAGGCTATCAGGTGGCTCCCGCTGAATTGGAATCCATCTTGCTCCAACA
CCCCAACATCTTCGACGCAGGTGTCGCAGGTCTTCCCGACGATGACGCCGGTGAACTTCCCG
CCGCCGTTGTTGTTTTGGAGCACGGAAAGACGATGACGGAAAAAGAGATCGTGGATTACGT
CGCCAGTCAAGTAACAACCGCGAAAAAGTTGCGCGGAGGAGTTGTGTTTGTGGACGAAGTA
CCGAAAGGTCTTACCGGAAAACTCGACGCAAGAAAAATCAGAGAGATCCTCATAAAGGCCA
AGAAGGGCGGAAAGATCGCCGTGTCAAGCCATGGCTTCCCGCCGGCGGTGGCGGCGCAGGA
TGATGGCACGCTGCCCATGTCTTGTGCCCAGGAGAGCGGGATGGACCGTCACCCTGCAGCCT
GTGCTTCTGCTAGGATCAATGTGTAGGCGGCCAACAATGCGATCCGATGGCCGCGACTCTAG
AGTCGGGGCGGCCGGCCGCTTCGAGCAGACATGATAAGATACATTGATGAGTTTGGACAAA
CCACAACTAGAATGCAGTGAAAAAAATGCTTTATTTGTGAAATTTGTGATGCTATTGCTTTAT
TTGTAACCATTATAAGCTGCAATAAACAAGTTAACAACAACAATTGCATTCATTTTATGTTTC
AGGTTCAGGGGGAGGTGTGGGAGGTTTTTTAAAGCAAGTAAAACCTCTACAAATGTGGTAA
AATCGATAAGCCCGTGCGGACCGAGCGGCCGCAGGAACCCCTAGTGATGGAGTTGGCCACT
CCCTCTCTGCGCGCTCGCTCGCTCACTGAGGCCGGGCGACCAAAGGTCGCCCGACGCCCGGG
CTTTGCCCGGGCGGCCTCAGTGAGCGAGCGAGCGCGCAGCTGCCTGCAG
pAAV-mBSEPpr-LucPEST
SEQ ID NO: 18
CCTGCAGGCAGCTGCGCGCTCGCTCGCTCACTGAGGCCGCCCGGGCGTCGGGCGACCTTTGG
TCGCCCGGCCTCAGTGAGCGAGCGAGCGCGCAGAGAGGGAGTGGCCAACTCCATCACTAGG
GGTTCCTGCGGCCGCACGCGTCATAGGTAGATCTTACCTCTTAGTTTCCCTTTCAGAAAGGA
GTGGTTATACGATAGAGTCTCTGAATGAAAAGTGAATAATAGTGATGAATGCCACTCCCAGG
CTTGGTCCATGGAAACTGTTCATGTAAAATACCCCATGTGATAGTTAGATGCCAATATCTAA
GGAGACCTTGAAAGCCGAATCTTCAGGATAACAGAGGCTTGTCATTCTAAGTCCCTGGATGA
CTATGGAGAACCTTCCTCCACTACAGACTTCTTCTCATGCAGAGCACAGTGGACTTCACGTG
AGGGTGATGAAGTTAGCAAGTGTCTTAGTTAGGGTTTCTATTCCTGCACAAAACATCATGAC
CACGAAGCAAGTTGGGGAGGAAAGGGTTTATTCAGCTTACATTTCCACATTGCTGTTCATCA
CCAAAAGAAGTCAGGACTGGAACTCACACAGGTCAGGAAGCAGAAGCGGATGCAGAGGCC
ATGGAGGGATGTTCCTTACTGGTTTGCTTCCCCTGGCTTGCTCAGCTTGCTTTCTTCTAGAAT
CCAAGACTACCAGCCCAGGGAAGGCACCACCCACAATGGGCCCTCCCCCCTTGATCACTAAT
TGAGAAAATGCCTTGCAGCTGGATCTTATGGAGGTCTTTCCTCAAGGGAGGTTCCTTTCTCTG
TATAACTCCAGCTTGTGTCAAGTTGACACACAAAACCAGCCAGTAGAGCAAGCATCATTACC
ATAGCTAAATCGAGAAGCAAGTGCTAGAACCTAATTATGAAGCCAGTTCCTTTTGCTTCTCT
CTGTAATGAGTTAAACCTCCCTACTCTACCAAAGTACAGCCAATTATGTTGTGTTAGATAATT
GGTGTTGAGTTTTTCCAATTCATTTTGATCTGCAGGGCTATAATAACCAAACAAGAGTTGAT
AGTTGGTGGAAGACTTCTCCACAAAACCATTCCTATTATTCTGAGCCACAGGGCTAACATTC
CAGTCTGTCTGCAATTCATCTCCTTGATTCTGAAAACTGTTCTTGGCCTACCTTCACCTTCTTA
GGTAAGCATGCATACCACTGTATTGTACCCTTTATGCTCAGTAGCCATCTCTGGTGTGGTGGG
TATGCTTTAAGAACCACAGTCTTGCAAGCTCAGCCTTTTTTCTTGTCCTCTCTCTGGCCTGGA
AGAAATGAAGATAGTGTAAAGAGATGCTATGTGTCTCTCCATCTGACCATCAGACTACTGTA
GTGAATCTGGTCCTCCCACCTCCAAATTGAAATTTCCTCAGGAATCTCACTCTGGGAGAAGA
AAGAGATACCAGCCAGAGCAAGAACTAGTCTCTTGAGACTTCACACAAGTCTCACAACTTG
GTAGACCACTTCAGGAACCAGCCTGAAGCCCAGTCAGTGGTGGGCAGGATCATATTTTAACT
GGCACTGCTAAGATGTGCATTTGGTGTTCTAATTAAATTCTGGTCTCATTTCAGGAGCCCAAA
ACTGGATATACACTGGATCTCTGTATTTTTTTCCCAATGGGAACACTGAATAAATCTCCTTTC
TCTGCTTTCTATTACTAACTTGGCTATTTTATTGGCCTATAGAAGGCAGCTGATCGGTTCTGA
CTTGGAGCACAAGTTGGGACCCCATGTCCAGTAACAATCTTACATCCCCTTTCCAGGAAGAA
ATATAAAAACTAAATAGCATTTTGAATCATATGGCCATATTACTTTTTATAATACAATGCAAT
TGAAATTAATGAAGAAGAAAAATGGTAAAACTTGTTTTTTTAAAGAAATATGTGGAGATTAA
TAGTAAGAAGGCATACGAAAAGTCACAGCCAGTTCTAAGCTTATTTGCTTTGAGGTTTTGTT
AATGGGAATGTGTTTTTTAAAGTAATGCATTTCTACTTATACAGCGCTGCAGCTAGTGGGCA
GAGCTTGAGGCTATTGACTGCTCAGACCAGAGTTCAAGGGTCATCTGTGTGCACAGAGTTCT
GAGCAGAAGGCAATTGCATGTTATCAATTCTCCATGAAACTTTGGGGCGGCTCCAGATCACT
GAGGGCCTGCAACCCAGGATGTCACAAGGGTCACTTCCAAATAGAGCAGAGACGATAAGAA
GTAGTTTGGTCCTGTTAGATTTTTTTAAAGTTACTTTTTAAAACCTAGGGCTTAAAAAAGAAG
AGTCGGGCCTCTCACCAGGCTCTCTACCATGAGGCCCCACTCTCGGGCAACTGGGCATTTTC
TAAAGCTTGTTGATACCCTCAGAAGGTCCCCACGCACTCTGGGTTTGGGTTCCTGCTTTGAGT
ATGTTCGACCTTTCCTCTCATGTCACTGAACTGTGCTAGATCTGGACTTTAGGCCATTGACCT
ATAAGCAAATAGATAGTGTTCTTAAAAAAGCCTGATTTCTGTTCAATGCTTTATTACCATGA
AAACTGAACTTGGAAAGGGGTGTACAACCCTGACTTTCCACAGTGGCGTCTCTCGCTTCTCC
TGGCTCCCTCAAATTCACAGCTAGCTTGGCATTCCGGTACTGTTGGTAAAGCCACCATGGAA
GACGCCAAAAACATAAAGAAAGGCCCGGCGCCATTCTATCCGCTGGAAGATGGAACCGCTG
GAGAGCAACTGCATAAGGCTATGAAGAGATACGCCCTGGTTCCTGGAACAATTGCTTTTACA
GATGCACATATCGAGGTGGACATCACTTACGCTGAGTACTTCGAAATGTCCGTTCGGTTGGC
AGAAGCTATGAAACGATATGGGCTGAATACAAATCACAGAATCGTCGTATGCAGTGAAAAC
TCTCTTCAATTCTTTATGCCGGTGTTGGGCGCGTTATTTATCGGAGTTGCAGTTGCGCCCGCG
AACGACATTTATAATGAACGTGAATTGCTCAACAGTATGGGCATTTCGCAGCCTACCGTGGT
GTTCGTTTCCAAAAAGGGGTTGCAAAAAATTTTGAACGTGCAAAAAAAGCTCCCAATCATCC
AAAAAATTATTATCATGGATTCTAAAACGGATTACCAGGGATTTCAGTCGATGTACACGTTC
GTCACATCTCATCTACCTCCCGGTTTTAATGAATACGATTTTGTGCCAGAGTCCTTCGATAGG
GACAAGACAATTGCACTGATCATGAACTCCTCTGGATCTACTGGTCTGCCTAAAGGTGTCGC
TCTGCCTCATAGAACTGCCTGCGTGAGATTCTCGCATGCCAGAGATCCTATTTTTGGCAATCA
AATCATTCCGGATACTGCGATTTTAAGTGTTGTTCCATTCCATCACGGTTTTGGAATGTTTAC
TACACTCGGATATTTGATATGTGGATTTCGAGTCGTCTTAATGTATAGATTTGAAGAAGAGC
TGTTTCTGAGGAGCCTTCAGGATTACAAGATTCAAAGTGCGCTGCTGGTGCCAACCCTATTC
TCCTTCTTCGCCAAAAGCACTCTGATTGACAAATACGATTTATCTAATTTACACGAAATTGCT
TCTGGTGGCGCTCCCCTCTCTAAGGAAGTCGGGGAAGCGGTTGCCAAGAGGTTCCATCTGCC
AGGTATCAGGCAAGGATATGGGCTCACTGAGACTACATCAGCTATTCTGATTACACCCGAGG
GGGATGATAAACCGGGCGCGGTCGGTAAAGTTGTTCCATTTTTTGAAGCGAAGGTTGTGGAT
CTGGATACCGGGAAAACGCTGGGCGTTAATCAAAGAGGCGAACTGTGTGTGAGAGGTCCTA
TGATTATGTCCGGTTATGTAAACAATCCGGAAGCGACCAACGCCTTGATTGACAAGGATGGA
TGGCTACATTCTGGAGACATAGCTTACTGGGACGAAGACGAACACTTCTTCATCGTTGACCG
CCTGAAGTCTCTGATTAAGTACAAAGGCTATCAGGTGGCTCCCGCTGAATTGGAATCCATCT
TGCTCCAACACCCCAACATCTTCGACGCAGGTGTCGCAGGTCTTCCCGACGATGACGCCGGT
GAACTTCCCGCCGCCGTTGTTGTTTTGGAGCACGGAAAGACGATGACGGAAAAAGAGATCG
TGGATTACGTCGCCAGTCAAGTAACAACCGCGAAAAAGTTGCGCGGAGGAGTTGTGTTTGTG
GACGAAGTACCGAAAGGTCTTACCGGAAAACTCGACGCAAGAAAAATCAGAGAGATCCTCA
TAAAGGCCAAGAAGGGCGGAAAGATCGCCGTGTCAAGCCATGGCTTCCCGCCGGCGGTGGC
GGCGCAGGATGATGGCACGCTGCCCATGTCTTGTGCCCAGGAGAGCGGGATGGACCGTCAC
CCTGCAGCCTGTGCTTCTGCTAGGATCAATGTGTAGGCGGCCAACAATGCGATCCGATGGCC
GCGACTCTAGAGTCGGGGCGGCCGGCCGCTTCGAGCAGACATGATAAGATACATTGATGAG
TTTGGACAAACCACAACTAGAATGCAGTGAAAAAAATGCTTTATTTGTGAAATTTGTGATGC
TATTGCTTTATTTGTAACCATTATAAGCTGCAATAAACAAGTTAACAACAACAATTGCATTC
ATTTTATGTTTCAGGTTCAGGGGGAGGTGTGGGAGGTTTTTTAAAGCAAGTAAAACCTCTAC
AAATGTGGTAAAATCGATAAGCCCGTGCGGACCGAGCGGCCGCAGGAACCCCTAGTGATGG
AGTTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGCTGCCTGCAG
imPr + 5xIR
SEQ ID NO: 19
TTAGGCCATTGACCTATTAGGCCATTGACCTATTAGGCCATTGACCTATTAGGCCAT
TGACCTATTAGGCCATTGACCTAGGTTCCTGCTTTGAGTATGTTCGACCTTTCCTCTC
ATGTCACTGAACTGTGCTAGATCTGGACTTTAGGCCATTGACCTATAAGCAAATAG
ATAGTGTTCTTAAAAAAGCCTGATTTCTGTTCAATGCTTTATTACCATGAAAACTGA
ACTTGGAAAGGGGTGTACAACCCTGACTTTCCACAGTGGCGTCTCTCGCTTCTCCTG
GCTCCCTCAAATTCACA
imPr + 3xIR-ER2
SEQ ID NO: 20
TGGACTTTAGGCCATTGACCTATGGACTTTAGGCCATTGACCTATGGACTTTAGGCC
ATTGACCTAGGTTCCTGCTTTGAGTATGTTCGACCTTTCCTCTCATGTCACTGAACTG
TGCTAGATCTGGACTTTAGGCCATTGACCTATAAGCAAATAGATAGTGTTCTTAAA
AAAGCCTGATTTCTGTTCAATGCTTTATTACCATGAAAACTGAACTTGGAAAGGGG
TGTACAACCCTGACTTTCCACAGTGGCGTCTCTCGCTTCTCCTGGCTCCCTCAAATT
CACA
imPr + LRH-1
SEQ ID NO: 21
TTTCTAAAGCTTGTTGATACCCTCAGAAGGTCCCCACGCACTCTGGGTTTGGGTTCC
TGCTTTGAGTATGTTCGACCTTTCCTCTCATGTCACTGAACTGTGCTAGATCTGGAC
TTTAGGCCATTGACCTATAAGCAAATAGATAGTGTTCTTAAAAAAGCCTGATTTCTG
TTCAATGCTTTATTACCATGAAAACTGAACTTGGAAAGGGGTGTACAACCCTGACT
TTCCACAGTGGCGTCTCTCGCTTCTCCTGGCTCCCTCAAATTCACA
pAAV-imPr + 1xIR-LucPEST
SEQ ID NO: 22
CCTGCAGGCAGCTGCGCGCTCGCTCGCTCACTGAGGCCGCCCGGGCGTCGGGCGAC
CTTTGGTCGCCCGGCCTCAGTGAGCGAGCGAGCGCGCAGAGAGGGAGTGGCCAAC
TCCATCACTAGGGGTTCCTGCGGCCGCACGCGTGGTACCTTAGGCCATTGACCTAG
GTTCCTGCTTTGAGTATGTTCGACCTTTCCTCTCATGTCACTGAACTGTGCTAGATCT
GGACTTTAGGCCATTGACCTATAAGCAAATAGATAGTGTTCTTAAAAAAGCCTGAT
TTCTGTTCAATGCTTTATTACCATGAAAACTGAACTTGGAAAGGGGTGTACAACCCT
GACTTTCCACAGTGGCGTCTCTCGCTTCTCCTGGCTCCCTCAAATTCACAGAGCTCG
TCGACGCTAGCTTGGCATTCCGGTACTGTTGGTAAAGCCACCATGGAAGACGCCAA
AAACATAAAGAAAGGCCCGGCGCCATTCTATCCGCTGGAAGATGGAACCGCTGGA
GAGCAACTGCATAAGGCTATGAAGAGATACGCCCTGGTTCCTGGAACAATTGCTTT
TACAGATGCACATATCGAGGTGGACATCACTTACGCTGAGTACTTCGAAATGTCCG
TTCGGTTGGCAGAAGCTATGAAACGATATGGGCTGAATACAAATCACAGAATCGTC
GTATGCAGTGAAAACTCTCTTCAATTCTTTATGCCGGTGTTGGGCGCGTTATTTATC
GGAGTTGCAGTTGCGCCCGCGAACGACATTTATAATGAACGTGAATTGCTCAACAG
TATGGGCATTTCGCAGCCTACCGTGGTGTTCGTTTCCAAAAAGGGGTTGCAAAAAA
TTTTGAACGTGCAAAAAAAGCTCCCAATCATCCAAAAAATTATTATCATGGATTCT
AAAACGGATTACCAGGGATTTCAGTCGATGTACACGTTCGTCACATCTCATCTACCT
CCCGGTTTTAATGAATACGATTTTGTGCCAGAGTCCTTCGATAGGGACAAGACAAT
TGCACTGATCATGAACTCCTCTGGATCTACTGGTCTGCCTAAAGGTGTCGCTCTGCC
TCATAGAACTGCCTGCGTGAGATTCTCGCATGCCAGAGATCCTATTTTTGGCAATCA
AATCATTCCGGATACTGCGATTTTAAGTGTTGTTCCATTCCATCACGGTTTTGGAAT
GTTTACTACACTCGGATATTTGATATGTGGATTTCGAGTCGTCTTAATGTATAGATT
TGAAGAAGAGCTGTTTCTGAGGAGCCTTCAGGATTACAAGATTCAAAGTGCGCTGC
TGGTGCCAACCCTATTCTCCTTCTTCGCCAAAAGCACTCTGATTGACAAATACGATT
TATCTAATTTACACGAAATTGCTTCTGGTGGCGCTCCCCTCTCTAAGGAAGTCGGGG
AAGCGGTTGCCAAGAGGTTCCATCTGCCAGGTATCAGGCAAGGATATGGGCTCACT
GAGACTACATCAGCTATTCTGATTACACCCGAGGGGGATGATAAACCGGGCGCGGT
CGGTAAAGTTGTTCCATTTTTTGAAGCGAAGGTTGTGGATCTGGATACCGGGAAAA
CGCTGGGCGTTAATCAAAGAGGCGAACTGTGTGTGAGAGGTCCTATGATTATGTCC
GGTTATGTAAACAATCCGGAAGCGACCAACGCCTTGATTGACAAGGATGGATGGCT
ACATTCTGGAGACATAGCTTACTGGGACGAAGACGAACACTTCTTCATCGTTGACC
GCCTGAAGTCTCTGATTAAGTACAAAGGCTATCAGGTGGCTCCCGCTGAATTGGAA
TCCATCTTGCTCCAACACCCCAACATCTTCGACGCAGGTGTCGCAGGTCTTCCCGAC
GATGACGCCGGTGAACTTCCCGCCGCCGTTGTTGTTTTGGAGCACGGAAAGACGAT
GACGGAAAAAGAGATCGTGGATTACGTCGCCAGTCAAGTAACAACCGCGAAAAAG
TTGCGCGGAGGAGTTGTGTTTGTGGACGAAGTACCGAAAGGTCTTACCGGAAAACT
CGACGCAAGAAAAATCAGAGAGATCCTCATAAAGGCCAAGAAGGGCGGAAAGATC
GCCGTGTCAAGCCATGGCTTCCCGCCGGCGGTGGCGGCGCAGGATGATGGCACGCT
GCCCATGTCTTGTGCCCAGGAGAGCGGGATGGACCGTCACCCTGCAGCCTGTGCTT
CTGCTAGGATCAATGTGTAGGCGGCCAACAATGCGATCCGATGGCCGCGACTCTAG
AGTCGGGGCGGCCGGCCGCTTCGAGCAGACATGATAAGATACATTGATGAGTTTGG
ACAAACCACAACTAGAATGCAGTGAAAAAAATGCTTTATTTGTGAAATTTGTGATG
CTATTGCTTTATTTGTAACCATTATAAGCTGCAATAAACAAGTTAACAACAACAATT
GCATTCATTTTATGTTTCAGGTTCAGGGGGAGGTGTGGGAGGTTTTTTAAAGCAAGT
AAAACCTCTACAAATGTGGTAAAATCGATAAGCCCGTGCGGACCGAGCGGCCGCA
GGAACCCCTAGTGATGGAGTTGGCCACTCCCTCTCTGCGCGCTCGCTCGCTCACTGA
GGCCGGGCGACCAAAGGTCGCCCGACGCCCGGGCTTTGCCCGGGCGGCCTCAGTGA
GCGAGCGAGCGCGCAGCTGCCTGCAG
pAAV-imPr + 3xIR-LucPEST
SEQ ID NO: 23
CCTGCAGGCAGCTGCGCGCTCGCTCGCTCACTGAGGCCGCCCGGGCGTCGGGCGAC
CTTTGGTCGCCCGGCCTCAGTGAGCGAGCGAGCGCGCAGAGAGGGAGTGGCCAAC
TCCATCACTAGGGGTTCCTGCGGCCGCACGCGTGGTACCTTAGGCCATTGACCTATT
AGGCCATTGACCTATTAGGCCATTGACCTAGGTTCCTGCTTTGAGTATGTTCGACCT
TTCCTCTCATGTCACTGAACTGTGCTAGATCTGGACTTTAGGCCATTGACCTATAAG
CAAATAGATAGTGTTCTTAAAAAAGCCTGATTTCTGTTCAATGCTTTATTACCATGA
AAACTGAACTTGGAAAGGGGTGTACAACCCTGACTTTCCACAGTGGCGTCTCTCGC
TTCTCCTGGCTCCCTCAAATTCACAGAGCTCgtcgacGCTAGCTTGGCATTCCGGTACT
GTTGGTAAAGCCACCATGGAAGACGCCAAAAACATAAAGAAAGGCCCGGCGCCAT
TCTATCCGCTGGAAGATGGAACCGCTGGAGAGCAACTGCATAAGGCTATGAAGAG
ATACGCCCTGGTTCCTGGAACAATTGCTTTTACAGATGCACATATCGAGGTGGACA
TCACTTACGCTGAGTACTTCGAAATGTCCGTTCGGTTGGCAGAAGCTATGAAACGA
TATGGGCTGAATACAAATCACAGAATCGTCGTATGCAGTGAAAACTCTCTTCAATT
CTTTATGCCGGTGTTGGGCGCGTTATTTATCGGAGTTGCAGTTGCGCCCGCGAACGA
CATTTATAATGAACGTGAATTGCTCAACAGTATGGGCATTTCGCAGCCTACCGTGGT
GTTCGTTTCCAAAAAGGGGTTGCAAAAAATTTTGAACGTGCAAAAAAAGCTCCCAA
TCATCCAAAAAATTATTATCATGGATTCTAAAACGGATTACCAGGGATTTCAGTCG
ATGTACACGTTCGTCACATCTCATCTACCTCCCGGTTTTAATGAATACGATTTTGTG
CCAGAGTCCTTCGATAGGGACAAGACAATTGCACTGATCATGAACTCCTCTGGATC
TACTGGTCTGCCTAAAGGTGTCGCTCTGCCTCATAGAACTGCCTGCGTGAGATTCTC
GCATGCCAGAGATCCTATTTTTGGCAATCAAATCATTCCGGATACTGCGATTTTAAG
TGTTGTTCCATTCCATCACGGTTTTGGAATGTTTACTACACTCGGATATTTGATATGT
GGATTTCGAGTCGTCTTAATGTATAGATTTGAAGAAGAGCTGTTTCTGAGGAGCCTT
CAGGATTACAAGATTCAAAGTGCGCTGCTGGTGCCAACCCTATTCTCCTTCTTCGCC
AAAAGCACTCTGATTGACAAATACGATTTATCTAATTTACACGAAATTGCTTCTGGT
GGCGCTCCCCTCTCTAAGGAAGTCGGGGAAGCGGTTGCCAAGAGGTTCCATCTGCC
AGGTATCAGGCAAGGATATGGGCTCACTGAGACTACATCAGCTATTCTGATTACAC
CCGAGGGGGATGATAAACCGGGCGCGGTCGGTAAAGTTGTTCCATTTTTTGAAGCG
AAGGTTGTGGATCTGGATACCGGGAAAACGCTGGGCGTTAATCAAAGAGGCGAAC
TGTGTGTGAGAGGTCCTATGATTATGTCCGGTTATGTAAACAATCCGGAAGCGACC
AACGCCTTGATTGACAAGGATGGATGGCTACATTCTGGAGACATAGCTTACTGGGA
CGAAGACGAACACTTCTTCATCGTTGACCGCCTGAAGTCTCTGATTAAGTACAAAG
GCTATCAGGTGGCTCCCGCTGAATTGGAATCCATCTTGCTCCAACACCCCAACATCT
TCGACGCAGGTGTCGCAGGTCTTCCCGACGATGACGCCGGTGAACTTCCCGCCGCC
GTTGTTGTTTTGGAGCACGGAAAGACGATGACGGAAAAAGAGATCGTGGATTACGT
CGCCAGTCAAGTAACAACCGCGAAAAAGTTGCGCGGAGGAGTTGTGTTTGTGGACG
AAGTACCGAAAGGTCTTACCGGAAAACTCGACGCAAGAAAAATCAGAGAGATCCT
CATAAAGGCCAAGAAGGGCGGAAAGATCGCCGTGTCAAGCCATGGCTTCCCGCCG
GCGGTGGCGGCGCAGGATGATGGCACGCTGCCCATGTCTTGTGCCCAGGAGAGCGG
GATGGACCGTCACCCTGCAGCCTGTGCTTCTGCTAGGATCAATGTGTAGGCGGCCAa
caatgcgatccgaTGGCCGCGACTCTAGAGTCGGGGCGGCCGGCCGCTTCGAGCAGACAT
GATAAGATACATTGATGAGTTTGGACAAACCACAACTAGAATGCAGTGAAAAAAA
TGCTTTATTTGTGAAATTTGTGATGCTATTGCTTTATTTGTAACCATTATAAGCTGCA
ATAAACAAGTTAACAACAACAATTGCATTCATTTTATGTTTCAGGTTCAGGGGGAG
GTGTGGGAGGTTTTTTAAAGCAAGTAAAACCTCTACAAATGTGGTAAAATCGATAA
GCCCGTGCGGACCGAGCGGCCGCAGGAACCCCTAGTGATGGAGTTGGCCACTCCCT
CTCTGCGCGCTCGCTCGCTCACTGAGGCCGGGCGACCAAAGGTCGCCCGACGCCCG
GGCTTTGCCCGGGCGGCCTCAGTGAGCGAGCGAGCGCGCAGCTGCCTGCAG
pAAV-imPr + 5xIR-LucPEST
SEQ ID NO: 24
CCTGCAGGCAGCTGCGCGCTCGCTCGCTCACTGAGGCCGCCCGGGCGTCGGGCGAC
CTTTGGTCGCCCGGCCTCAGTGAGCGAGCGAGCGCGCAGAGAGGGAGTGGCCAAC
TCCATCACTAGGGGTTCCTGCGGCCGCACGCGTGGTACCTTAGGCCATTGACCTATT
AGGCCATTGACCTATTAGGCCATTGACCTATTAGGCCATTGACCTATTAGGCCATTG
ACCTAGGTTCCTGCTTTGAGTATGTTCGACCTTTCCTCTCATGTCACTGAACTGTGCT
AGATCTGGACTTTAGGCCATTGACCTATAAGCAAATAGATAGTGTTCTTAAAAAAG
CCTGATTTCTGTTCAATGCTTTATTACCATGAAAACTGAACTTGGAAAGGGGTGTAC
AACCCTGACTTTCCACAGTGGCGTCTCTCGCTTCTCCTGGCTCCCTCAAATTCACAG
AGCTCGTCGACGCTAGCTTGGCATTCCGGTACTGTTGGTAAAGCCACCATGGAAGA
CGCCAAAAACATAAAGAAAGGCCCGGCGCCATTCTATCCGCTGGAAGATGGAACC
GCTGGAGAGCAACTGCATAAGGCTATGAAGAGATACGCCCTGGTTCCTGGAACAAT
TGCTTTTACAGATGCACATATCGAGGTGGACATCACTTACGCTGAGTACTTCGAAAT
GTCCGTTCGGTTGGCAGAAGCTATGAAACGATATGGGCTGAATACAAATCACAGAA
TCGTCGTATGCAGTGAAAACTCTCTTCAATTCTTTATGCCGGTGTTGGGCGCGTTAT
TTATCGGAGTTGCAGTTGCGCCCGCGAACGACATTTATAATGAACGTGAATTGCTC
AACAGTATGGGCATTTCGCAGCCTACCGTGGTGTTCGTTTCCAAAAAGGGGTTGCA
AAAAATTTTGAACGTGCAAAAAAAGCTCCCAATCATCCAAAAAATTATTATCATGG
ATTCTAAAACGGATTACCAGGGATTTCAGTCGATGTACACGTTCGTCACATCTCATC
TACCTCCCGGTTTTAATGAATACGATTTTGTGCCAGAGTCCTTCGATAGGGACAAGA
CAATTGCACTGATCATGAACTCCTCTGGATCTACTGGTCTGCCTAAAGGTGTCGCTC
TGCCTCATAGAACTGCCTGCGTGAGATTCTCGCATGCCAGAGATCCTATTTTTGGCA
ATCAAATCATTCCGGATACTGCGATTTTAAGTGTTGTTCCATTCCATCACGGTTTTG
GAATGTTTACTACACTCGGATATTTGATATGTGGATTTCGAGTCGTCTTAATGTATA
GATTTGAAGAAGAGCTGTTTCTGAGGAGCCTTCAGGATTACAAGATTCAAAGTGCG
CTGCTGGTGCCAACCCTATTCTCCTTCTTCGCCAAAAGCACTCTGATTGACAAATAC
GATTTATCTAATTTACACGAAATTGCTTCTGGTGGCGCTCCCCTCTCTAAGGAAGTC
GGGGAAGCGGTTGCCAAGAGGTTCCATCTGCCAGGTATCAGGCAAGGATATGGGCT
CACTGAGACTACATCAGCTATTCTGATTACACCCGAGGGGGATGATAAACCGGGCG
CGGTCGGTAAAGTTGTTCCATTTTTTGAAGCGAAGGTTGTGGATCTGGATACCGGG
AAAACGCTGGGCGTTAATCAAAGAGGCGAACTGTGTGTGAGAGGTCCTATGATTAT
GTCCGGTTATGTAAACAATCCGGAAGCGACCAACGCCTTGATTGACAAGGATGGAT
GGCTACATTCTGGAGACATAGCTTACTGGGACGAAGACGAACACTTCTTCATCGTT
GACCGCCTGAAGTCTCTGATTAAGTACAAAGGCTATCAGGTGGCTCCCGCTGAATT
GGAATCCATCTTGCTCCAACACCCCAACATCTTCGACGCAGGTGTCGCAGGTCTTCC
CGACGATGACGCCGGTGAACTTCCCGCCGCCGTTGTTGTTTTGGAGCACGGAAAGA
CGATGACGGAAAAAGAGATCGTGGATTACGTCGCCAGTCAAGTAACAACCGCGAA
AAAGTTGCGCGGAGGAGTTGTGTTTGTGGACGAAGTACCGAAAGGTCTTACCGGAA
AACTCGACGCAAGAAAAATCAGAGAGATCCTCATAAAGGCCAAGAAGGGCGGAAA
GATCGCCGTGTCAAGCCATGGCTTCCCGCCGGCGGTGGCGGCGCAGGATGATGGCA
CGCTGCCCATGTCTTGTGCCCAGGAGAGCGGGATGGACCGTCACCCTGCAGCCTGT
GCTTCTGCTAGGATCAATGTGTAGGCGGCCAACAATGCGATCCGATGGCCGCGACT
CTAGAGTCGGGGCGGCCGGCCGCTTCGAGCAGACATGATAAGATACATTGATGAGT
TTGGACAAACCACAACTAGAATGCAGTGAAAAAAATGCTTTATTTGTGAAATTTGT
GATGCTATTGCTTTATTTGTAACCATTATAAGCTGCAATAAACAAGTTAACAACAAC
AATTGCATTCATTTTATGTTTCAGGTTCAGGGGGAGGTGTGGGAGGTTTTTTAAAGC
AAGTAAAACCTCTACAAATGTGGTAAAATCGATAAGCCCGTGCGGACCGAGCGGC
CGCAGGAACCCCTAGTGATGGAGTTGGCCACTCCCTCTCTGCGCGCTCGCTCGCTCA
CTGAGGCCGGGCGACCAAAGGTCGCCCGACGCCCGGGCTTTGCCCGGGCGGCCTCA
GTGAGCGAGCGAGCGCGCAGCTGCCTGCAG
pAAV-imPr + 3xIR-ER2-LucPEST
SEQ ID NO: 25
CCTGCAGGCAGCTGCGCGCTCGCTCGCTCACTGAGGCCGCCCGGGCGTCGGGCGAC
CTTTGGTCGCCCGGCCTCAGTGAGCGAGCGAGCGCGCAGAGAGGGAGTGGCCAAC
TCCATCACTAGGGGTTCCTGCGGCCGCACGCGTGGTACCTGGACTTTAGGCCATTG
ACCTATGGACTTTAGGCCATTGACCTATGGACTTTAGGCCATTGACCTAGGTTCCTG
CTTTGAGTATGTTCGACCTTTCCTCTCATGTCACTGAACTGTGCTAGATCTGGACTTT
AGGCCATTGACCTATAAGCAAATAGATAGTGTTCTTAAAAAAGCCTGATTTCTGTTC
AATGCTTTATTACCATGAAAACTGAACTTGGAAAGGGGTGTACAACCCTGACTTTC
CACAGTGGCGTCTCTCGCTTCTCCTGGCTCCCTCAAATTCACAGAGCTCGTCGACGC
TAGCTTGGCATTCCGGTACTGTTGGTAAAGCCACCATGGAAGACGCCAAAAACATA
AAGAAAGGCCCGGCGCCATTCTATCCGCTGGAAGATGGAACCGCTGGAGAGCAAC
TGCATAAGGCTATGAAGAGATACGCCCTGGTTCCTGGAACAATTGCTTTTACAGAT
GCACATATCGAGGTGGACATCACTTACGCTGAGTACTTCGAAATGTCCGTTCGGTT
GGCAGAAGCTATGAAACGATATGGGCTGAATACAAATCACAGAATCGTCGTATGC
AGTGAAAACTCTCTTCAATTCTTTATGCCGGTGTTGGGCGCGTTATTTATCGGAGTT
GCAGTTGCGCCCGCGAACGACATTTATAATGAACGTGAATTGCTCAACAGTATGGG
CATTTCGCAGCCTACCGTGGTGTTCGTTTCCAAAAAGGGGTTGCAAAAAATTTTGA
ACGTGCAAAAAAAGCTCCCAATCATCCAAAAAATTATTATCATGGATTCTAAAACG
GATTACCAGGGATTTCAGTCGATGTACACGTTCGTCACATCTCATCTACCTCCCGGT
TTTAATGAATACGATTTTGTGCCAGAGTCCTTCGATAGGGACAAGACAATTGCACT
GATCATGAACTCCTCTGGATCTACTGGTCTGCCTAAAGGTGTCGCTCTGCCTCATAG
AACTGCCTGCGTGAGATTCTCGCATGCCAGAGATCCTATTTTTGGCAATCAAATCAT
TCCGGATACTGCGATTTTAAGTGTTGTTCCATTCCATCACGGTTTTGGAATGTTTACT
ACACTCGGATATTTGATATGTGGATTTCGAGTCGTCTTAATGTATAGATTTGAAGAA
GAGCTGTTTCTGAGGAGCCTTCAGGATTACAAGATTCAAAGTGCGCTGCTGGTGCC
AACCCTATTCTCCTTCTTCGCCAAAAGCACTCTGATTGACAAATACGATTTATCTAA
TTTACACGAAATTGCTTCTGGTGGCGCTCCCCTCTCTAAGGAAGTCGGGGAAGCGG
TTGCCAAGAGGTTCCATCTGCCAGGTATCAGGCAAGGATATGGGCTCACTGAGACT
ACATCAGCTATTCTGATTACACCCGAGGGGGATGATAAACCGGGCGCGGTCGGTAA
AGTTGTTCCATTTTTTGAAGCGAAGGTTGTGGATCTGGATACCGGGAAAACGCTGG
GCGTTAATCAAAGAGGCGAACTGTGTGTGAGAGGTCCTATGATTATGTCCGGTTAT
GTAAACAATCCGGAAGCGACCAACGCCTTGATTGACAAGGATGGATGGCTACATTC
TGGAGACATAGCTTACTGGGACGAAGACGAACACTTCTTCATCGTTGACCGCCTGA
AGTCTCTGATTAAGTACAAAGGCTATCAGGTGGCTCCCGCTGAATTGGAATCCATC
TTGCTCCAACACCCCAACATCTTCGACGCAGGTGTCGCAGGTCTTCCCGACGATGA
CGCCGGTGAACTTCCCGCCGCCGTTGTTGTTTTGGAGCACGGAAAGACGATGACGG
AAAAAGAGATCGTGGATTACGTCGCCAGTCAAGTAACAACCGCGAAAAAGTTGCG
CGGAGGAGTTGTGTTTGTGGACGAAGTACCGAAAGGTCTTACCGGAAAACTCGACG
CAAGAAAAATCAGAGAGATCCTCATAAAGGCCAAGAAGGGCGGAAAGATCGCCGT
GTCAAGCCATGGCTTCCCGCCGGCGGTGGCGGCGCAGGATGATGGCACGCTGCCCA
TGTCTTGTGCCCAGGAGAGCGGGATGGACCGTCACCCTGCAGCCTGTGCTTCTGCT
AGGATCAATGTGTAGGCGGCCAACAATGCGATCCGATGGCCGCGACTCTAGAGTCG
GGGCGGCCGGCCGCTTCGAGCAGACATGATAAGATACATTGATGAGTTTGGACAAA
CCACAACTAGAATGCAGTGAAAAAAATGCTTTATTTGTGAAATTTGTGATGCTATT
GCTTTATTTGTAACCATTATAAGCTGCAATAAACAAGTTAACAACAACAATTGCATT
CATTTTATGTTTCAGGTTCAGGGGGAGGTGTGGGAGGTTTTTTAAAGCAAGTAAAA
CCTCTACAAATGTGGTAAAATCGATAAGCCCGTGCGGACCGAGCGGCCGCAGGAA
CCCCTAGTGATGGAGTTGGCCACTCCCTCTCTGCGCGCTCGCTCGCTCACTGAGGCC
GGGCGACCAAAGGTCGCCCGACGCCCGGGCTTTGCCCGGGCGGCCTCAGTGAGCG
AGCGAGCGCGCAGCTGCCTGCAG
pAAV-imPr + LRH-1-LucPEST
SEQ ID NO: 26
CCTGCAGGCAGCTGCGCGCTCGCTCGCTCACTGAGGCCGCCCGGGCGTCGGGCGAC
CTTTGGTCGCCCGGCCTCAGTGAGCGAGCGAGCGCGCAGAGAGGGAGTGGCCAAC
TCCATCACTAGGGGTTCCTGCGGCCGCACGCGTGGTACCTTTCTAAAGCTTGTTGAT
ACCCTCAGAAGGTCCCCACGCACTCTGGGTTTGGGTTCCTGCTTTGAGTATGTTCGA
CCTTTCCTCTCATGTCACTGAACTGTGCTAGATCTGGACTTTAGGCCATTGACCTAT
AAGCAAATAGATAGTGTTCTTAAAAAAGCCTGATTTCTGTTCAATGCTTTATTACCA
TGAAAACTGAACTTGGAAAGGGGTGTACAACCCTGACTTTCCACAGTGGCGTCTCT
CGCTTCTCCTGGCTCCCTCAAATTCACAGAGCTCgtcgacGCTAGCTTGGCATTCCGGT
ACTGTTGGTAAAGCCACCATGGAAGACGCCAAAAACATAAAGAAAGGCCCGGCGC
CATTCTATCCGCTGGAAGATGGAACCGCTGGAGAGCAACTGCATAAGGCTATGAAG
AGATACGCCCTGGTTCCTGGAACAATTGCTTTTACAGATGCACATATCGAGGTGGA
CATCACTTACGCTGAGTACTTCGAAATGTCCGTTCGGTTGGCAGAAGCTATGAAAC
GATATGGGCTGAATACAAATCACAGAATCGTCGTATGCAGTGAAAACTCTCTTCAA
TTCTTTATGCCGGTGTTGGGCGCGTTATTTATCGGAGTTGCAGTTGCGCCCGCGAAC
GACATTTATAATGAACGTGAATTGCTCAACAGTATGGGCATTTCGCAGCCTACCGT
GGTGTTCGTTTCCAAAAAGGGGTTGCAAAAAATTTTGAACGTGCAAAAAAAGCTCC
CAATCATCCAAAAAATTATTATCATGGATTCTAAAACGGATTACCAGGGATTTCAG
TCGATGTACACGTTCGTCACATCTCATCTACCTCCCGGTTTTAATGAATACGATTTT
GTGCCAGAGTCCTTCGATAGGGACAAGACAATTGCACTGATCATGAACTCCTCTGG
ATCTACTGGTCTGCCTAAAGGTGTCGCTCTGCCTCATAGAACTGCCTGCGTGAGATT
CTCGCATGCCAGAGATCCTATTTTTGGCAATCAAATCATTCCGGATACTGCGATTTT
AAGTGTTGTTCCATTCCATCACGGTTTTGGAATGTTTACTACACTCGGATATTTGAT
ATGTGGATTTCGAGTCGTCTTAATGTATAGATTTGAAGAAGAGCTGTTTCTGAGGA
GCCTTCAGGATTACAAGATTCAAAGTGCGCTGCTGGTGCCAACCCTATTCTCCTTCT
TCGCCAAAAGCACTCTGATTGACAAATACGATTTATCTAATTTACACGAAATTGCTT
CTGGTGGCGCTCCCCTCTCTAAGGAAGTCGGGGAAGCGGTTGCCAAGAGGTTCCAT
CTGCCAGGTATCAGGCAAGGATATGGGCTCACTGAGACTACATCAGCTATTCTGAT
TACACCCGAGGGGGATGATAAACCGGGCGCGGTCGGTAAAGTTGTTCCATTTTTTG
AAGCGAAGGTTGTGGATCTGGATACCGGGAAAACGCTGGGCGTTAATCAAAGAGG
CGAACTGTGTGTGAGAGGTCCTATGATTATGTCCGGTTATGTAAACAATCCGGAAG
CGACCAACGCCTTGATTGACAAGGATGGATGGCTACATTCTGGAGACATAGCTTAC
TGGGACGAAGACGAACACTTCTTCATCGTTGACCGCCTGAAGTCTCTGATTAAGTA
CAAAGGCTATCAGGTGGCTCCCGCTGAATTGGAATCCATCTTGCTCCAACACCCCA
ACATCTTCGACGCAGGTGTCGCAGGTCTTCCCGACGATGACGCCGGTGAACTTCCC
GCCGCCGTTGTTGTTTTGGAGCACGGAAAGACGATGACGGAAAAAGAGATCGTGG
ATTACGTCGCCAGTCAAGTAACAACCGCGAAAAAGTTGCGCGGAGGAGTTGTGTTT
GTGGACGAAGTACCGAAAGGTCTTACCGGAAAACTCGACGCAAGAAAAATCAGAG
AGATCCTCATAAAGGCCAAGAAGGGCGGAAAGATCGCCGTGTCAAGCCATGGCTT
CCCGCCGGCGGTGGCGGCGCAGGATGATGGCACGCTGCCCATGTCTTGTGCCCAGG
AGAGCGGGATGGACCGTCACCCTGCAGCCTGTGCTTCTGCTAGGATCAATGTGTAG
GCGGCCAacaatgcgatccgaTGGCCGCGACTCTAGAGTCGGGGCGGCCGGCCGCTTCGAG
CAGACATGATAAGATACATTGATGAGTTTGGACAAACCACAACTAGAATGCAGTGA
AAAAAATGCTTTATTTGTGAAATTTGTGATGCTATTGCTTTATTTGTAACCATTATA
AGCTGCAATAAACAAGTTAACAACAACAATTGCATTCATTTTATGTTTCAGGTTCAG
GGGGAGGTGTGGGAGGTTTTTTAAAGCAAGTAAAACCTCTACAAATGTGGTAAAAT
CGATAAGCCCGTGCGGACCGAGCGGCCGCAGGAACCCCTAGTGATGGAGTTGGCC
ACTCCCTCTCTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCAGCTGCCTGC
AG

1. A nucleic acid construct comprising:
a bile acid inducible promoter having a length of less than 500 bp and comprising or consisting of nucleic acid sequence SEQ ID NO: 1 or a functional variant thereof having at least 95% identity to SEQ ID NO: 1 operably linked to a therapeutic transgene.

2. The nucleic acid construct of claim 1 comprising a poly(A) signal sequence of SEQ ID NO: 10.

3. The nucleic acid construct of claim 1 comprising 5′ITR and 3′ITR sequences of an adeno-associated virus (AAV.

4. The nucleic acid construct according claim 1, wherein said bile acid inducible promoter is the only eukaryotic regulatory element sequence preceding said therapeutic transgene in said nucleic acid construct.

5. The nucleic acid construct according to claim 1, wherein said promoter further comprises at least one murine IR-1 element of SEQ ID NO: 3.

6. The nucleic acid construct according to claim 1 wherein said promoter has a length of less than 450 bp.

7. The nucleic acid construct according to claim 1, wherein said promoter is operably linked to a transgene encoding for human BSEP.

8. The nucleic acid construct according to claim 1, wherein said promoter is operably linked to a transgene encoding for human MDR3 protein.

9. An expression vector comprising a nucleic acid construct according to claim 1.

10. A viral particle comprising a nucleic acid construct according to claim 1.

11. An AAV particle comprising a nucleic acid construct according to claim 1.

12. A host cell comprising a nucleic acid construct according to claim 1.

13. A pharmaceutical composition comprising the nucleic acid construct according to claim 1 and a pharmaceutically acceptable excipient.

14. (canceled)

15. A method for the treatment of cholestatic diseases in a subject in need thereof comprising administering to the subject a therapeutically effective amount of the nucleic acid construct according to claim 1.

16. A method of producing viral particles comprising the nucleic acid construct of claim 1, comprising the steps of:
a) culturing a host cell comprising the nucleic acid construct of claim 1 in a culture medium, and
b) harvesting the viral particles from the cell culture supernatant and/or inside the host cells.

17. The nucleic acid construct according to claim 1 wherein said promoter comprises or consists of a sequence SEQ ID NO: 6 or 7.

18. The nucleic acid construct according to claim 1 wherein said promoter is operably linked to a transgene comprising or consisting of SEQ ID NO: 8 or a variant having at least 80% identity to SEQ ID NO:8.

19. The nucleic acid construct according to claim 1 wherein said promoter is operably linked to a transgene comprising or consisting of SEQ ID NO: 9 or a variant having at least 80% identity to SEQ ID NO:9.

20. An AAV particle comprising a nucleic acid construct according to claim 1 and a capsid protein of adeno-associated virus selected from the group consisting of: AAV3 type 3A, AAV3 type 3B, NP40, NP59, NP84, LK03, AAV3-ST, Anc80 and AAV8 serotype.

21. A method for the treatment of Progressive Familial Intrahepatic Cholestasis Type 2 (PFIC2) or Progressive Familial Intrahepatic Cholestasis Type 3 (PFIC3) in a subject in need thereof comprising administering to the subject a therapeutically effective amount of the nucleic acid construct according to claim 1.


SEQUENCE LISTING (26 sequences)

SEQ ID NO: 1 - DNA, 222 bp, Artificial sequence
  Synthetic murine minimal bile acid inducible promoter
ggttcctgctttgagtatgttcgacctttcctctcatgtcactgaactgtgctagatctggactttaggccattgacctataagcaaatagatagtgttcttaaaaaagcctgatttctgttcaatgctttattaccatgaaaactgaacttggaaaggggtgtacaaccctgactttccacagtggcgtctctcgcttctcctggctccctcaaattcaca

SEQ ID NO: 2 - DNA, 231 bp, Artificial sequence
  Synthetic human minimal bile acid inducible promoter
ttcccaagcacactctgtgtttggggttattgctctgagtatgtttctcgtatgtcactgaactgtgcttgggctgcccttagggacattgatccttaggcaaatagataatgttcttgaaaaagtttgaattctgttcagtgctttagaatgatgaaaaccgaggttggaaaaggttgtgaaaccttttaactctccacagtggagtccattatttcctctggcttcctc

SEQ ID NO: 3 - DNA, 16 bp, Artificial sequence
  Synthetic murine IR-1 element
ttaggccattgaccta

SEQ ID NO: 4 - DNA, 22 bp, Artificial sequence
  Synthetic IR-1-ER2 element
tggactttaggccattgaccta

SEQ ID NO: 5 - DNA, 11 bp, Artificial sequence
  Synthetic LRHRE
tttctaaagct

SEQ ID NO: 6 - DNA, 238 bp, Artificial sequence
  Synthetic imPR+1xIR
ttaggccattgacctaggttcctgctttgagtatgttcgacctttcctctcatgtcactgaactgtgctagatctggactttaggccattgacctataagcaaatagatagtgttcttaaaaaagcctgatttctgttcaatgctttattaccatgaaaactgaacttggaaaggggtgtacaaccctgactttccacagtggcgtctctcgcttctcctggctccctcaaattcaca

SEQ ID NO: 7 - DNA, 270 bp, Artificial sequence
  Synthetic imPR+3xIR
ttaggccattgacctattaggccattgacctattaggccattgacctaggttcctgctttgagtatgttcgacctttcctctcatgtcactgaactgtgctagatctggactttaggccattgacctataagcaaatagatagtgttcttaaaaaagcctgatttctgttcaatgctttattaccatgaaaactgaacttggaaaggggtgtacaaccctgactttccacagtggcgtctctcgcttctcctggctccctcaaattcaca

SEQ ID NO: 8 - DNA, 3966 bp, Artificial sequence
  Synthetic codon-optimized version of ABCB11 cDNA
atgagcgactccgtgattctgagatcaatcaaaaaattcggcgaagaaaatgacgggttcgagagcgataaatcctataataatgacaagaagtctaggctgcaggacgagaagaagggcgatggcgtgcgggtgggcttctttcagctgttccggttcagcagcagcaccgacatctggctgatgtttgtgggcagcctgtgcgccttcctgcacggcatcgcacagccaggcgtgctgctgatctttggcaccatgacagacgtgttcatcgactacgatgtggagctgcaggagctgcagatccctggcaaagcctgcgtgaacaataccatcgtgtggacaaacagctccctgaaccagaatatgaccaacggcacacgctgtggcctgctgaatatcgagtctgagatgatcaagtttgccagctactatgcaggaatcgcagtggccgtgctgatcaccggctacatccagatttgcttctgggtcatcgcagcagcaaggcagatccagaagatgagaaagttctattttcggagaatcatgcggatggagatcggctggtttgactgtaactctgtgggcgagctgaatacaagattcagcgacgacatcaacaagatcaatgacgccatcgccgatcagatggccctgtttatccagcggatgaccagcacaatctgtggcttcctgctgggcttctttagaggctggaagctgaccctggtcatcatcagcgtgtccccactgatcggaatcggagcagcaacaatcggcctgtctgtgagcaagttcaccgactacgagctgaaagcctacgccaaggcaggagtggtggcagatgaagtgatcagcagcatgaggaccgtggcagcctttggcggagagaagagggaggtggagcggtacgagaagaacctggtgttcgcccagcggtggggcatcagaaagggcatcgtgatgggcttctttacaggcttcgtgtggtgcctgatcttcctgtgctacgccctggccttttggtatggctccaccctggtgctggacgagggagagtatacccctggcacactggtgcagattttcctgagcgtgatcgtgggcgccctgaacctgggaaatgcatccccatgcctggaagccttcgccacaggaagggcagcagccacctccatcttcgagacaatcgaccgcaagcctatcatcgactgtatgtctgaggatggctacaagctggacaggatcaagggcgagatcgagtttcacaatgtgaccttccactatcccagccgccctgaggtgaagatcctgaacgatctgaatatggtcatcaagccaggagagatgaccgccctggtgggaccctctggagcaggcaagagcaccgccctgcagctgatccagcggttttacgacccttgcgagggaatggtgaccgtggacggacacgacatcaggtccctgaacatccagtggctgcgcgatcagatcggcatcgtggagcaggagccagtgctgttctctaccacaatcgccgagaatatcagatacggccgcgaggatgccacaatggaggacatcgtgcaggccgccaaggaggccaacgcctataacttcatcatggatctgccccagcagttcgacaccctggtgggagagggaggaggacagatgtccggaggccagaagcagagagtggccatcgccagagccctgatccgcaaccctaagatcctgctgctggatatggccacaagcgccctggacaatgagtccgaggctatggtgcaggaggtgctgagcaagatccagcacggccacaccatcatctctgtggcacacaggctgagcacagtgagagcagccgacaccatcatcggctttgagcacggcacagcagtggagaggggcacccacgaggagctgctggagaggaagggcgtgtacttcaccctggtgacactgcagtcccagggcaaccaggccctgaatgaggaggacatcaaggatgccacagaggacgatatgctggcccggaccttcagcagaggctcctatcaggattctctgagggccagcatcaggcagcggagcaagtctcagctgagctacctggtgcacgagccacctctggcagtggtggaccacaagtccacctatgaggaggatcgcaaggacaaggacatcccagtgcaggaggaggtggagcctgcaccagtgaggcgcatcctgaagttttccgccccagagtggccctacatgctggtgggatctgtgggagcagcagtgaacggcaccgtgacaccactgtatgccttcctgttttcccagatcctgggcaccttctctatccccgacaaggaggagcagcggtcccagatcaatggcgtgtgcctgctgtttgtggctatgggctgcgtgagcctgtttacacagttcctgcagggctacgccttcgccaagagcggcgagctgctgaccaagcggctgagaaagttcggctttagagccatgctgggccaggacatcgcctggtttgacgatctgcggaacagcccaggcgccctgaccacaagactggccacagatgcatctcaggtgcagggagcagcaggcagccagatcggcatgatcgtgaactccttcaccaatgtgacagtggccatgatcatcgccttcagcttttcctggaagctgagcctggtcatcctgtgcttcttcccctttctggccctgagcggagcaacccagacaaggatgctgaccggcttcgcctccagagacaagcaggccctggagatggtgggccagatcacaaacgaggccctgagcaatatcaggaccgtggcaggaatcggcaaggagcggcggttcatcgaggccctggagacagagctggagaagcctttcaagaccgccatccagaaggccaacatctacggcttctgctttgccttcgcccagtgtatcatgttcatcgccaactctgccagctaccgctatggcggctacctgatcagcaatgagggcctgcacttcagctacgtgttcagagtgatcagcgccgtggtgctgtctgccacagccctgggaagggccttctcctacaccccatcttatgccaaggccaagatcagcgccgccaggttctttcagctgctggaccgccagccacccatcagcgtgtacaacacagccggcgagaagtgggataatttccagggcaagatcgactttgtggattgcaagttcacctatcctagcagaccagactcccaggtgctgaatggcctgtccgtgtctatcagcccaggccagacactggcctttgtgggctcctctggctgtggcaagtccacctctatccagctgctggagcggttctatgaccccgatcagggcaaagtgatgatcgacggccacgatagcaagaaggtgaacgtgcagtttctgagatccaatatcggcatcgtgtctcaggagcctgtgctgttcgcctgctccatcatggataacatcaagtacggcgacaatacaaaggagatcccaatggagagagtgatcgcagcagcaaagcaggcacagctgcacgatttcgtgatgtccctgcccgagaagtatgagacaaacgtgggctctcagggcagccagctgtccaggggcgagaagcagaggatcgcaatcgccagggccatcgtgcgcgatcccaagatcctgctgctggacgaggccaccagcgccctggatacagagtccgagaagaccgtgcaggtggccctggacaaggcccgggagggaagaacatgtatcgtgatcgcccacagactgagcaccatccagaatgccgacatcatcgccgtgatggcccagggcgtggtcatcgagaagggcacccacgaggaactgatggcacagaaaggggcttactacaaactggtcacaacaggctcacctatctcatag

SEQ ID NO: 9 - DNA, 3840 bp, artificial sequence
  Synthetic human codon-optimized version of ABCB4 cDNA
atggatctggaggccgccaagaacggcaccgcctggagacccacaagcgccgagggcgacttcgagctgggcatcagctccaagcagaagagaaagaagaccaagacagtgaagatgatcggcgtgctgacactgttcaggtactccgactggcaggataagctgtttatgtctctgggcaccatcatggcaatcgcccacggcagcggcctgcctctgatgatgatcgtgttcggcgagatgaccgacaagtttgtggatacagccggcaatttctcctttcccgtgaacttctctctgagcctgctgaaccctggcaagatcctggaggaggagatgacaagatatgcctactattactctggcctgggagcaggcgtgctggtggcagcatacatccaggtgagcttctggaccctggcagcaggccggcagatcagaaagatcaggcagaagttctttcacgccatcctgcgccaggagatcggctggtttgacatcaatgataccacagagctgaacacccggctgacagacgacatctctaagatcagcgagggcatcggcgataaagtgggcatgttctttcaggccgtggccacattctttgccggcttcatcgtgggctttatcaggggctggaagctgaccctggtcatcatggccatctctccaatcctgggcctgagcgccgccgtgtgggcaaagatcctgtccgccttctctgacaaggagctggccgcctacgccaaggcaggagcagtggcagaggaggccctgggcgccatccgcaccgtgatcgcctttggcggccagaataaggagctggagcggtatcagaagcacctggagaacgccaaggagatcggcatcaagaaggccatctccgccaatatctctatgggcatcgccttcctgctgatctatgccagctacgccctggccttttggtatggcagcaccctggtcatcagcaaggagtacaccatcggcaatgccatgacagtgttctttagcatcctgatcggagccttctccgtgggacaggcagcaccatgcatcgacgccttcgccaacgccagaggcgcagcctacgtgatctttgacatcatcgataacaatccaaagatcgactccttttctgagcggggccacaagcccgattccatcaagggcaatctggagttcaacgacgtgcactttagctatccctcccgcgccaatgtgaagatcctgaagggcctgaacctgaaggtgcagtccggacagacagtggccctggtgggctctagcggatgcggcaagtctaccacagtgcagctgatccagcggctgtatgacccagatgagggcaccatcaacatcgacggccaggacatccgcaacttcaatgtgaactacctgcgggagatcatcggcgtggtgtctcaggagcccgtgctgtttagcaccacaatcgccgagaatatctgttatggccgcggcaacgtgacaatggatgagatcaagaaggccgtgaaggaggccaatgcctacgagttcatcatgaagctgccccagaagtttgacaccctggtgggagagagaggcgcccagctgagcggaggccagaagcagaggatcgccatcgccagagccctggtgaggaaccctaagatcctgctgctggacgaggccacctccgccctggatacagagtctgaggcagaggtgcaggccgccctggataaggcccgcgagggacggaccacaatcgtgatcgcccacagactgagcacagtgaggaatgccgacgtgatcgccggcttcgaggatggcgtgatcgtggagcagggcagccactccgagctgatgaagaaggagggcgtgtacttcaagctggtgaacatgcagacctctggcagccagatccagtccgaggagtttgagctgaatgacgagaaggcagcaacaaggatggcacctaacggatggaagagcagactgttcaggcactccacccagaagaatctgaagaactctcagatgtgccagaagagcctggacgtggaaaccgatggactggaggcaaatgtgccacccgtgagcttcctgaaggtgctgaagctgaacaagacagagtggccatattttgtggtgggcaccgtgtgcgcaatcgcaaatggcggcctgcagccagccttcagcgtgatcttttccgagatcatcgccatcttcggccctggcgacgatgccgtgaagcagcagaagtgtaacatcttttccctgatcttcctgtttctgggcatcatctctttctttaccttctttctgcagggcttcacatttggcaaggccggcgagatcctgacacggagactgagaagcatggccttcaaggccatgctgaggcaggatatgtcctggtttgacgatcacaagaacagcaccggcgccctgtccaccagactggcaacagacgcagcacaggtgcagggagcaaccggcacaaggctggccctgatcgcccagaatatcgccaacctgggcacaggcatcatcatctctttcatctacggctggcagctgaccctgctgctgctggcagtggtgccaatcatcgccgtgagcggcatcgtggagatgaagctgctggccggcaatgccaagagagacaagaaggagctggaggcagcaggcaagatcgcaaccgaggccatcgagaacatccgcaccgtggtgagcctgacacaggagcggaagttcgagtccatgtatgtggagaagctgtatggcccttaccgcaattccgtgcagaaggcccacatctacggcatcaccttttccatctctcaggctttcatgtatttttcttacgccggctgcttccggtttggcgcctatctgatcgtgaacggccacatgaggttccgcgatgtgatcctggtgttctctgccatcgtgtttggagcagtggccctgggacacgcctcctcttttgcccctgactatgcaaaggcaaagctgtccgccgcacacctgttcatgctgtttgagagacagcctctgatcgatagctactccgaggagggcctgaagccagacaagttcgagggcaatatcacattcaacgaggtggtgtttaattatccaaccagggccaacgtgcccgtgctgcagggcctgagcctggaggtgaagaagggacagacactggccctggtgggcagctccggatgcggcaagtccaccgtggtgcagctgctggagagattctacgaccctctggcaggcaccgtgctgctggatggacaggaggccaagaagctgaatgtgcagtggctgagagcccagctgggcatcgtgtctcaggagccaatcctgttcgattgtagcatcgccgagaatatcgcctacggcgacaactctagggtggtgagccaggatgagatcgtgagcgccgcaaaggcagcaaacatccacccttttatcgagaccctgccacacaagtatgagacacgcgtgggcgacaagggcacccagctgtccggaggacagaagcagaggatcgcaatcgcccgcgccctgatcaggcagccccagatcctgctgctggatgaagccacctccgccctggacacagagtctgagaaggtggtgcaggaggccctggacaaagcccgcgagggacggacatgtattgtcattgctcacagactgagcaccatccagaatgccgacctgatcgtggtgttccagaacggcagggtgaaggagcacggcacacaccagcagctgctggcccagaagggcatctacttttctatggtgagcgtgcaggccggcacccagaacctgtag

SEQ ID NO: 10 - DNA, 56 bp, Artificial sequence
  Synthetic polyA
aataaagacctcttattttcattcatcaggtgtggttggtttttttgtgtgggggc

SEQ ID NO: 11 - DNA, 130 bp, Artificial sequence
  Synthetic 5′ITR AAV2
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 12 - DNA, 141 bp, Artificial sequence
  Synthetic 3′ITR AAV2
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 13 - DNA, 4606 bp, Artificial sequence
  Synthetic AAV-imPr-hBSEPco
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaattccatggtaccggttcctgctttgagtatgttcgacctttcctctcatgtcactgaactgtgctagatctggactttaggccattgacctataagcaaatagatagtgttcttaaaaaagcctgatttctgttcaatgctttattaccatgaaaactgaacttggaaaggggtgtacaaccctgactttccacagtggcgtctctcgcttctcctggctccctcaaattcacagagctcgccgccaccatgagcgactccgtgattctgagatcaatcaaaaaattcggcgaagaaaatgacgggttcgagagcgataaatcctataataatgacaagaagtctaggctgcaggacgagaagaagggcgatggcgtgcgggtgggcttctttcagctgttccggttcagcagcagcaccgacatctggctgatgtttgtgggcagcctgtgcgccttcctgcacggcatcgcacagccaggcgtgctgctgatctttggcaccatgacagacgtgttcatcgactacgatgtggagctgcaggagctgcagatccctggcaaagcctgcgtgaacaataccatcgtgtggacaaacagctccctgaaccagaatatgaccaacggcacacgctgtggcctgctgaatatcgagtctgagatgatcaagtttgccagctactatgcaggaatcgcagtggccgtgctgatcaccggctacatccagatttgcttctgggtcatcgcagcagcaaggcagatccagaagatgagaaagttctattttcggagaatcatgcggatggagatcggctggtttgactgtaactctgtgggcgagctgaatacaagattcagcgacgacatcaacaagatcaatgacgccatcgccgatcagatggccctgtttatccagcggatgaccagcacaatctgtggcttcctgctgggcttctttagaggctggaagctgaccctggtcatcatcagcgtgtccccactgatcggaatcggagcagcaacaatcggcctgtctgtgagcaagttcaccgactacgagctgaaagcctacgccaaggcaggagtggtggcagatgaagtgatcagcagcatgaggaccgtggcagcctttggcggagagaagagggaggtggagcggtacgagaagaacctggtgttcgcccagcggtggggcatcagaaagggcatcgtgatgggcttctttacaggcttcgtgtggtgcctgatcttcctgtgctacgccctggccttttggtatggctccaccctggtgctggacgagggagagtatacccctggcacactggtgcagattttcctgagcgtgatcgtgggcgccctgaacctgggaaatgcatccccatgcctggaagccttcgccacaggaagggcagcagccacctccatcttcgagacaatcgaccgcaagcctatcatcgactgtatgtctgaggatggctacaagctggacaggatcaagggcgagatcgagtttcacaatgtgaccttccactatcccagccgccctgaggtgaagatcctgaacgatctgaatatggtcatcaagccaggagagatgaccgccctggtgggaccctctggagcaggcaagagcaccgccctgcagctgatccagcggttttacgacccttgcgagggaatggtgaccgtggacggacacgacatcaggtccctgaacatccagtggctgcgcgatcagatcggcatcgtggagcaggagccagtgctgttctctaccacaatcgccgagaatatcagatacggccgcgaggatgccacaatggaggacatcgtgcaggccgccaaggaggccaacgcctataacttcatcatggatctgccccagcagttcgacaccctggtgggagagggaggaggacagatgtccggaggccagaagcagagagtggccatcgccagagccctgatccgcaaccctaagatcctgctgctggatatggccacaagcgccctggacaatgagtccgaggctatggtgcaggaggtgctgagcaagatccagcacggccacaccatcatctctgtggcacacaggctgagcacagtgagagcagccgacaccatcatcggctttgagcacggcacagcagtggagaggggcacccacgaggagctgctggagaggaagggcgtgtacttcaccctggtgacactgcagtcccagggcaaccaggccctgaatgaggaggacatcaaggatgccacagaggacgatatgctggcccggaccttcagcagaggctcctatcaggattctctgagggccagcatcaggcagcggagcaagtctcagctgagctacctggtgcacgagccacctctggcagtggtggaccacaagtccacctatgaggaggatcgcaaggacaaggacatcccagtgcaggaggaggtggagcctgcaccagtgaggcgcatcctgaagttttccgccccagagtggccctacatgctggtgggatctgtgggagcagcagtgaacggcaccgtgacaccactgtatgccttcctgttttcccagatcctgggcaccttctctatccccgacaaggaggagcagcggtcccagatcaatggcgtgtgcctgctgtttgtggctatgggctgcgtgagcctgtttacacagttcctgcagggctacgccttcgccaagagcggcgagctgctgaccaagcggctgagaaagttcggctttagagccatgctgggccaggacatcgcctggtttgacgatctgcggaacagcccaggcgccctgaccacaagactggccacagatgcatctcaggtgcagggagcagcaggcagccagatcggcatgatcgtgaactccttcaccaatgtgacagtggccatgatcatcgccttcagcttttcctggaagctgagcctggtcatcctgtgcttcttcccctttctggccctgagcggagcaacccagacaaggatgctgaccggcttcgcctccagagacaagcaggccctggagatggtgggccagatcacaaacgaggccctgagcaatatcaggaccgtggcaggaatcggcaaggagcggcggttcatcgaggccctggagacagagctggagaagcctttcaagaccgccatccagaaggccaacatctacggcttctgctttgccttcgcccagtgtatcatgttcatcgccaactctgccagctaccgctatggcggctacctgatcagcaatgagggcctgcacttcagctacgtgttcagagtgatcagcgccgtggtgctgtctgccacagccctgggaagggccttctcctacaccccatcttatgccaaggccaagatcagcgccgccaggttctttcagctgctggaccgccagccacccatcagcgtgtacaacacagccggcgagaagtgggataatttccagggcaagatcgactttgtggattgcaagttcacctatcctagcagaccagactcccaggtgctgaatggcctgtccgtgtctatcagcccaggccagacactggcctttgtgggctcctctggctgtggcaagtccacctctatccagctgctggagcggttctatgaccccgatcagggcaaagtgatgatcgacggccacgatagcaagaaggtgaacgtgcagtttctgagatccaatatcggcatcgtgtctcaggagcctgtgctgttcgcctgctccatcatggataacatcaagtacggcgacaatacaaaggagatcccaatggagagagtgatcgcagcagcaaagcaggcacagctgcacgatttcgtgatgtccctgcccgagaagtatgagacaaacgtgggctctcagggcagccagctgtccaggggcgagaagcagaggatcgcaatcgccagggccatcgtgcgcgatcccaagatcctgctgctggacgaggccaccagcgccctggatacagagtccgagaagaccgtgcaggtggccctggacaaggcccgggagggaagaacatgtatcgtgatcgcccacagactgagcaccatccagaatgccgacatcatcgccgtgatggcccagggcgtggtcatcgagaagggcacccacgaggaactgatggcacagaaaggggcttactacaaactggtcacaacaggctcacctatctcatagggatccatatgatatcaataaagacctcttattttcattcatcaggtgtggttggtttttttgtgtgggggctcgagatctgaggaacccctagtgatggaggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 14 - DNA, 4487 bp, Artificial sequence
  Synthetic AAV-imPr-MDR3co
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaattccatggtaccggttcctgctttgagtatgttcgacctttcctctcatgtcactgaactgtgctagatctggactttaggccattgacctataagcaaatagatagtgttcttaaaaaagcctgatttctgttcaatgctttattaccatgaaaactgaacttggaaaggggtgtacaaccctgactttccacagtggcgtctctcgcttctcctggctccctcaaattcacagagctcgtcgacggatccgccgccaccatggatctggaggccgccaagaacggcaccgcctggagacccacaagcgccgagggcgacttcgagctgggcatcagctccaagcagaagagaaagaagaccaagacagtgaagatgatcggcgtgctgacactgttcaggtactccgactggcaggataagctgtttatgtctctgggcaccatcatggcaatcgcccacggcagcggcctgcctctgatgatgatcgtgttcggcgagatgaccgacaagtttgtggatacagccggcaatttctcctttcccgtgaacttctctctgagcctgctgaaccctggcaagatcctggaggaggagatgacaagatatgcctactattactctggcctgggagcaggcgtgctggtggcagcatacatccaggtgagcttctggaccctggcagcaggccggcagatcagaaagatcaggcagaagttctttcacgccatcctgcgccaggagatcggctggtttgacatcaatgataccacagagctgaacacccggctgacagacgacatctctaagatcagcgagggcatcggcgataaagtgggcatgttctttcaggccgtggccacattctttgccggcttcatcgtgggctttatcaggggctggaagctgaccctggtcatcatggccatctctccaatcctgggcctgagcgccgccgtgtgggcaaagatcctgtccgccttctctgacaaggagctggccgcctacgccaaggcaggagcagtggcagaggaggccctgggcgccatccgcaccgtgatcgcctttggcggccagaataaggagctggagcggtatcagaagcacctggagaacgccaaggagatcggcatcaagaaggccatctccgccaatatctctatgggcatcgccttcctgctgatctatgccagctacgccctggccttttggtatggcagcaccctggtcatcagcaaggagtacaccatcggcaatgccatgacagtgttctttagcatcctgatcggagccttctccgtgggacaggcagcaccatgcatcgacgccttcgccaacgccagaggcgcagcctacgtgatctttgacatcatcgataacaatccaaagatcgactccttttctgagcggggccacaagcccgattccatcaagggcaatctggagttcaacgacgtgcactttagctatccctcccgcgccaatgtgaagatcctgaagggcctgaacctgaaggtgcagtccggacagacagtggccctggtgggctctagcggatgcggcaagtctaccacagtgcagctgatccagcggctgtatgacccagatgagggcaccatcaacatcgacggccaggacatccgcaacttcaatgtgaactacctgcgggagatcatcggcgtggtgtctcaggagcccgtgctgtttagcaccacaatcgccgagaatatctgttatggccgcggcaacgtgacaatggatgagatcaagaaggccgtgaaggaggccaatgcctacgagttcatcatgaagctgccccagaagtttgacaccctggtgggagagagaggcgcccagctgagcggaggccagaagcagaggatcgccatcgccagagccctggtgaggaaccctaagatcctgctgctggacgaggccacctccgccctggatacagagtctgaggcagaggtgcaggccgccctggataaggcccgcgagggacggaccacaatcgtgatcgcccacagactgagcacagtgaggaatgccgacgtgatcgccggcttcgaggatggcgtgatcgtggagcagggcagccactccgagctgatgaagaaggagggcgtgtacttcaagctggtgaacatgcagacctctggcagccagatccagtccgaggagtttgagctgaatgacgagaaggcagcaacaaggatggcacctaacggatggaagagcagactgttcaggcactccacccagaagaatctgaagaactctcagatgtgccagaagagcctggacgtggaaaccgatggactggaggcaaatgtgccacccgtgagcttcctgaaggtgctgaagctgaacaagacagagtggccatattttgtggtgggcaccgtgtgcgcaatcgcaaatggcggcctgcagccagccttcagcgtgatcttttccgagatcatcgccatcttcggccctggcgacgatgccgtgaagcagcagaagtgtaacatcttttccctgatcttcctgtttctgggcatcatctctttctttaccttctttctgcagggcttcacatttggcaaggccggcgagatcctgacacggagactgagaagcatggccttcaaggccatgctgaggcaggatatgtcctggtttgacgatcacaagaacagcaccggcgccctgtccaccagactggcaacagacgcagcacaggtgcagggagcaaccggcacaaggctggccctgatcgcccagaatatcgccaacctgggcacaggcatcatcatctctttcatctacggctggcagctgaccctgctgctgctggcagtggtgccaatcatcgccgtgagcggcatcgtggagatgaagctgctggccggcaatgccaagagagacaagaaggagctggaggcagcaggcaagatcgcaaccgaggccatcgagaacatccgcaccgtggtgagcctgacacaggagcggaagttcgagtccatgtatgtggagaagctgtatggcccttaccgcaattccgtgcagaaggcccacatctacggcatcaccttttccatctctcaggctttcatgtatttttcttacgccggctgcttccggtttggcgcctatctgatcgtgaacggccacatgaggttccgcgatgtgatcctggtgttctctgccatcgtgtttggagcagtggccctgggacacgcctcctcttttgcccctgactatgcaaaggcaaagctgtccgccgcacacctgttcatgctgtttgagagacagcctctgatcgatagctactccgaggagggcctgaagccagacaagttcgagggcaatatcacattcaacgaggtggtgtttaattatccaaccagggccaacgtgcccgtgctgcagggcctgagcctggaggtgaagaagggacagacactggccctggtgggcagctccggatgcggcaagtccaccgtggtgcagctgctggagagattctacgaccctctggcaggcaccgtgctgctggatggacaggaggccaagaagctgaatgtgcagtggctgagagcccagctgggcatcgtgtctcaggagccaatcctgttcgattgtagcatcgccgagaatatcgcctacggcgacaactctagggtggtgagccaggatgagatcgtgagcgccgcaaaggcagcaaacatccacccttttatcgagaccctgccacacaagtatgagacacgcgtgggcgacaagggcacccagctgtccggaggacagaagcagaggatcgcaatcgcccgcgccctgatcaggcagccccagatcctgctgctggatgaagccacctccgccctggacacagagtctgagaaggtggtgcaggaggccctggacaaagcccgcgagggacggacatgtattgtcattgctcacagactgagcaccatccagaatgccgacctgatcgtggtgttccagaacggcagggtgaaggagcacggcacacaccagcagctgctggcccagaagggcatctacttttctatggtgagcgtgcaggccggcacccagaacctgtagcatatgatatcaataaagacctcttattttcattcatcaggtgtggttggtttttttgtgtgggggctcgagatctgaggaacccctagtgatggaggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 15 - DNA, 14 bp, Artificial sequence
  Synthetic human IR-1 element
gggacattgatcct

SEQ ID NO: 16 - DNA, 2662 bp, Artificial sequence
  Synthetic pAAV-ihPr-LucPEST
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtaccttcccaagcacactctgtgtttggggttattgctctgagtatgtttctcgtatgtcactgaactgtgcttgggctgcccttagggacattgatccttaggcaaatagataatgttcttgaaaaagtttgaattctgttcagtgctttagaatgatgaaaaccgaggttggaaaaggttgtgaaaccttttaactctccacagtggagtccattatttcctctggcttcctcgagctcgtcgacgctagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtcaagccatggcttcccgccggcggtggcggcgcaggatgatggcacgctgcccatgtcttgtgcccaggagagcgggatggaccgtcaccctgcagcctgtgcttctgctaggatcaatgtgtaggcggccaacaatgcgatccgatggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataagcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 17 - DNA, 2653 bp, Artificial sequence
  Synthetic pAAV-imPr-LucPEST
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtaccggttcctgctttgagtatgttcgacctttcctctcatgtcactgaactgtgctagatctggactttaggccattgacctataagcaaatagatagtgttcttaaaaaagcctgatttctgttcaatgctttattaccatgaaaactgaacttggaaaggggtgtacaaccctgactttccacagtggcgtctctcgcttctcctggctccctcaaattcacagagctcgtcgacgctagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtcaagccatggcttcccgccggcggtggcggcgcaggatgatggcacgctgcccatgtcttgtgcccaggagagcgggatggaccgtcaccctgcagcctgtgcttctgctaggatcaatgtgtaggcggccaacaatgcgatccgatggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataagcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 18 - DNA, 4901 bp, Artificial sequence
  Synthetic pAAV-mBSEPpr-LucPEST
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcataggtagatcttacctcttagtttccctttcagaaaggagtggttatacgatagagtctctgaatgaaaagtgaataatagtgatgaatgccactcccaggcttggtccatggaaactgttcatgtaaaataccccatgtgatagttagatgccaatatctaaggagaccttgaaagccgaatcttcaggataacagaggcttgtcattctaagtccctggatgactatggagaaccttcctccactacagacttcttctcatgcagagcacagtggacttcacgtgagggtgatgaagttagcaagtgtcttagttagggtttctattcctgcacaaaacatcatgaccacgaagcaagttggggaggaaagggtttattcagcttacatttccacattgctgttcatcaccaaaagaagtcaggactggaactcacacaggtcaggaagcagaagcggatgcagaggccatggagggatgttccttactggtttgcttcccctggcttgctcagcttgctttcttctagaatccaagactaccagcccagggaaggcaccacccacaatgggccctccccccttgatcactaattgagaaaatgccttgcagctggatcttatggaggtctttcctcaagggaggttcctttctctgtataactccagcttgtgtcaagttgacacacaaaaccagccagtagagcaagcatcattaccatagctaaatcgagaagcaagtgctagaacctaattatgaagccagttccttttgcttctctctgtaatgagttaaacctccctactctaccaaagtacagccaattatgttgtgttagataattggtgttgagtttttccaattcattttgatctgcagggctataataaccaaacaagagttgatagttggtggaagacttctccacaaaaccattcctattattctgagccacagggctaacattccagtctgtctgcaattcatctccttgattctgaaaactgttcttggcctaccttcaccttcttaggtaagcatgcataccactgtattgtaccctttatgctcagtagccatctctggtgtggtgggtatgctttaagaaccacagtcttgcaagctcagccttttttcttgtcctctctctggcctggaagaaatgaagatagtgtaaagagatgctatgtgtctctccatctgaccatcagactactgtagtgaatctggtcctcccacctccaaattgaaatttcctcaggaatctcactctgggagaagaaagagataccagccagagcaagaactagtctcttgagacttcacacaagtctcacaacttggtagaccacttcaggaaccagcctgaagcccagtcagtggtgggcaggatcatattttaactggcactgctaagatgtgcatttggtgttctaattaaattctggtctcatttcaggagcccaaaactggatatacactggatctctgtatttttttcccaatgggaacactgaataaatctcctttctctgctttctattactaacttggctattttattggcctatagaaggcagctgatcggttctgacttggagcacaagttgggaccccatgtccagtaacaatcttacatcccctttccaggaagaaatataaaaactaaatagcattttgaatcatatggccatattactttttataatacaatgcaattgaaattaatgaagaagaaaaatggtaaaacttgtttttttaaagaaatatgtggagattaatagtaagaaggcatacgaaaagtcacagccagttctaagcttatttgctttgaggttttgttaatgggaatgtgttttttaaagtaatgcatttctacttatacagcgctgcagctagtgggcagagcttgaggctattgactgctcagaccagagttcaagggtcatctgtgtgcacagagttctgagcagaaggcaattgcatgttatcaattctccatgaaactttggggcggctccagatcactgagggcctgcaacccaggatgtcacaagggtcacttccaaatagagcagagacgataagaagtagtttggtcctgttagatttttttaaagttactttttaaaacctagggcttaaaaaagaagagtcgggcctctcaccaggctctctaccatgaggccccactctcgggcaactgggcattttctaaagcttgttgataccctcagaaggtccccacgcactctgggtttgggttcctgctttgagtatgttcgacctttcctctcatgtcactgaactgtgctagatctggactttaggccattgacctataagcaaatagatagtgttcttaaaaaagcctgatttctgttcaatgctttattaccatgaaaactgaacttggaaaggggtgtacaaccctgactttccacagtggcgtctctcgcttctcctggctccctcaaattcacagctagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtcaagccatggcttcccgccggcggtggcggcgcaggatgatggcacgctgcccatgtcttgtgcccaggagagcgggatggaccgtcaccctgcagcctgtgcttctgctaggatcaatgtgtaggcggccaacaatgcgatccgatggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataagcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 19 - DNA, 302 bp, Artificial sequence
  Synthetic imPR+5xIR
ttaggccattgacctattaggccattgacctattaggccattgacctattaggccattgacctattaggccattgacctaggttcctgctttgagtatgttcgacctttcctctcatgtcactgaactgtgctagatctggactttaggccattgacctataagcaaatagatagtgttcttaaaaaagcctgatttctgttcaatgctttattaccatgaaaactgaacttggaaaggggtgtacaaccctgactttccacagtggcgtctctcgcttctcctggctccctcaaattcaca

SEQ ID NO: 20 - DNA, 288 bp, Artificial sequence
  Synthetic imPr+3xIR-ER2
tggactttaggccattgacctatggactttaggccattgacctatggactttaggccattgacctaggttcctgctttgagtatgttcgacctttcctctcatgtcactgaactgtgctagatctggactttaggccattgacctataagcaaatagatagtgttcttaaaaaagcctgatttctgttcaatgctttattaccatgaaaactgaacttggaaaggggtgtacaaccctgactttccacagtggcgtctctcgcttctcctggctccctcaaattcaca

SEQ ID NO: 21 - DNA, 273 bp, Artificial sequence
  Synthetic imPr+ LRH-1
tttctaaagcttgttgataccctcagaaggtccccacgcactctgggtttgggttcctgctttgagtatgttcgacctttcctctcatgtcactgaactgtgctagatctggactttaggccattgacctataagcaaatagatagtgttcttaaaaaagcctgatttctgttcaatgctttattaccatgaaaactgaacttggaaaggggtgtacaaccctgactttccacagtggcgtctctcgcttctcctggctccctcaaattcaca

SEQ ID NO: 22 - DNA, 2669 bp, Artificial sequence
  Synthetic pAAV-imPr+1xIR-LucPEST
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtaccttaggccattgacctaggttcctgctttgagtatgttcgacctttcctctcatgtcactgaactgtgctagatctggactttaggccattgacctataagcaaatagatagtgttcttaaaaaagcctgatttctgttcaatgctttattaccatgaaaactgaacttggaaaggggtgtacaaccctgactttccacagtggcgtctctcgcttctcctggctccctcaaattcacagagctcgtcgacgctagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtcaagccatggcttcccgccggcggtggcggcgcaggatgatggcacgctgcccatgtcttgtgcccaggagagcgggatggaccgtcaccctgcagcctgtgcttctgctaggatcaatgtgtaggcggccaacaatgcgatccgatggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataagcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 23 - DNA, 2701 bp, Artificial sequence
  Synthetic pAAV-imPr+3xIR-LucPEST
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtaccttaggccattgacctattaggccattgacctattaggccattgacctaggttcctgctttgagtatgttcgacctttcctctcatgtcactgaactgtgctagatctggactttaggccattgacctataagcaaatagatagtgttcttaaaaaagcctgatttctgttcaatgctttattaccatgaaaactgaacttggaaaggggtgtacaaccctgactttccacagtggcgtctctcgcttctcctggctccctcaaattcacagagctcgtcgacgctagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtcaagccatggcttcccgccggcggtggcggcgcaggatgatggcacgctgcccatgtcttgtgcccaggagagcgggatggaccgtcaccctgcagcctgtgcttctgctaggatcaatgtgtaggcggccaacaatgcgatccgatggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataagcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 24 - DNA, 2733 bp, Artificial sequence
  Synthetic pAAV-imPr+3xIR-LucPEST
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtaccttaggccattgacctattaggccattgacctattaggccattgacctattaggccattgacctattaggccattgacctaggttcctgctttgagtatgttcgacctttcctctcatgtcactgaactgtgctagatctggactttaggccattgacctataagcaaatagatagtgttcttaaaaaagcctgatttctgttcaatgctttattaccatgaaaactgaacttggaaaggggtgtacaaccctgactttccacagtggcgtctctcgcttctcctggctccctcaaattcacagagctcgtcgacgctagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtcaagccatggcttcccgccggcggtggcggcgcaggatgatggcacgctgcccatgtcttgtgcccaggagagcgggatggaccgtcaccctgcagcctgtgcttctgctaggatcaatgtgtaggcggccaacaatgcgatccgatggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataagcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 25 - DNA, 2719 bp, Artificial sequence
  Synthetic pAAV-imPr+3xIR-ER2-LucPEST
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtacctggactttaggccattgacctatggactttaggccattgacctatggactttaggccattgacctaggttcctgctttgagtatgttcgacctttcctctcatgtcactgaactgtgctagatctggactttaggccattgacctataagcaaatagatagtgttcttaaaaaagcctgatttctgttcaatgctttattaccatgaaaactgaacttggaaaggggtgtacaaccctgactttccacagtggcgtctctcgcttctcctggctccctcaaattcacagagctcgtcgacgctagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtcaagccatggcttcccgccggcggtggcggcgcaggatgatggcacgctgcccatgtcttgtgcccaggagagcgggatggaccgtcaccctgcagcctgtgcttctgctaggatcaatgtgtaggcggccaacaatgcgatccgatggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataagcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 26 - DNA, 2704 bp, Artificial sequence
  Synthetic pAAV-imPr+LRH-1-LucPEST
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggtacctttctaaagcttgttgataccctcagaaggtccccacgcactctgggtttgggttcctgctttgagtatgttcgacctttcctctcatgtcactgaactgtgctagatctggactttaggccattgacctataagcaaatagatagtgttcttaaaaaagcctgatttctgttcaatgctttattaccatgaaaactgaacttggaaaggggtgtacaaccctgactttccacagtggcgtctctcgcttctcctggctccctcaaattcacagagctcgtcgacgctagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtcaagccatggcttcccgccggcggtggcggcgcaggatgatggcacgctgcccatgtcttgtgcccaggagagcgggatggaccgtcaccctgcagcctgtgcttctgctaggatcaatgtgtaggcggccaacaatgcgatccgatggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataagcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag